(19) United States
(12) Patent Application Publication
(10) Pub. No.: US 2006/0035920 A1
(43) Pub. Date: Feb. 16, 2006

(54) Chk-1 inhibitors

(75) Inventors: Robert George Boyle, Cambridge (GB); Hassan Julien Imogai, Geneva (CH); Michael Cherry, Haddenham (GB); Alfred John Humphries, Saffron Walden (GB); Eva Figueroa Navarro, Cambridge (GB); David Rodney Owen, Cambridge (GB); Natalie A. Dales, Arlington, MA (US); Matthew J. LaMarche, Reading, MA (US); Courtney Cullis, Bedford, MA (US); Alexandra E. Gould, Cambridge, MA (US); Paul D. Greenspan, Acton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Millennium Pharmaceuticals, Inc., Cambridge, MA (US)

(21) Appl. No.: 11/140,553

(22) Filed: May 27, 2005

Related U.S. Application Data
(60) Provisional application No. 60/575,159, filed on May 28, 2004.
(60) Provisional application No. 60/634,359, filed on Dec. 8, 2004.
(60) Provisional application No. 60/634,360, filed on Dec. 8, 2004.

(51) Int. Cl. A61K 31/4745; C07D 471/14

(52) U.S. Cl. 514/292; 546/82

(57) ABSTRACT

Disclosed are novel inhibitors of Chk-1 and methods of using the same for therapy.

### RELATED APPLICATIONS

This application claims the benefit of U.S. Provisional Application Nos. 60/575,159, filed on May 28, 2004, 60/634,359, filed on Dec. 8, 2004, and 60/634,360, filed on Dec. 8, 2004. The entire teachings of the above applications are incorporated herein by reference.

### BACKGROUND OF THE INVENTION

Cell cycle checkpoints are regulatory pathways that control the order and timing of cell cycle transitions. They ensure that critical events such as DNA replication and chromosome segregation are completed in high fidelity. The regulation of these cell cycle checkpoints is a critical determinant of the manner in which tumor cells respond to many chemotherapies and radiation. Many effective cancer therapies work by causing DNA damage; however, resistance to these agents remains a significant limitation in the treatment of cancer. There are several mechanisms of drug resistance: an important one is attributed to the prevention of cell cycle progression through the control of critical activation of a checkpoint pathway that arrests the cell cycle to provide time for repair and induces the transcription of genes to facilitate repair, thereby avoiding immediate cell death. By abrogating checkpoint arrests at, for example, the G2 checkpoint, it may be possible to synergistically augment tumor cell death induced by DNA damage and circumvent resistance. (Shyjan et al., U.S. Pat. No. 6,723,498 (2004)). Human Chk-1 plays a role in regulating cell cycle arrest by phosphorylating the phosphatase cdc25 on Serine 216, which may be involved in preventing activation of cdc2/cyclin B and initiating mitosis. (Sanchez et al., Science, 277:1497 (1997)). Therefore, inhibition of Chk-1 should enhance DNA damaging agents by initiating mitosis before DNA repair is complete and thereby causing tumor cell death.

### SUMMARY OF THE INVENTION

It has now been found that certain 2,5-dihydro-pyrazolo[4,3-c]quinolin-4-ones are effective inhibitors of Chk-1. For example, compounds as described in Example 57 have IC₅₀ values less than 1 μM when tested in an in vitro assay that assesses the inhibitory activity of test compounds. Based on these discoveries, novel Chk-1 inhibitors, methods of inhibiting Chk-1 in a subject and methods of treating cancer are disclosed herein.

In one embodiment the present invention is a Chk-1 inhibitor represented by Structural Formula (I):

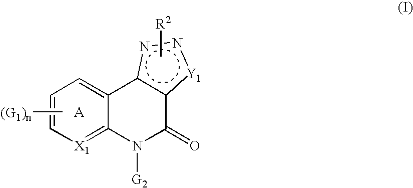

Ring A is optionally substituted at any one or more substitutable ring carbon atoms.

Y₁ is N or CR³.

G₂ is —H, or a C1-C8 aliphatic group optionally substituted with one or more fluoro, —OR¹², —CONR¹¹R¹², —COOR¹², cycloalkyl or phenyl, wherein the cycloalkyl and phenyl are optionally substituted with halo or alkyl.

R² is —H or a group that is cleavable in vivo.

R³ is —H, halogen, alkyl, haloalkyl or -V₁-R⁷, wherein V₁ is a covalent bond or a C1-C4 alkylidene optionally substituted with one or more —OR¹⁴, —NR¹⁵R¹⁶, alkyl, hydroxyalkyl, alkoxyalkyl, aminoalkyl, or with a spiro cycloalkyl group; R⁷ is —OR¹⁴, —SR¹⁴, —CONR¹⁵R¹⁶, —NR¹⁵R¹⁶, —NHC(O)NR¹⁵R¹⁶, —CN, —COOR¹⁴, —NHC(O)H, —NHC(O)R¹⁴, —OC(O)R¹⁴, —OC(O)NR¹⁵R¹⁶, —NHC(O)—OR¹⁴, —S(O)₂NR¹⁵R¹⁶, —S(O)₂(R¹⁴), boronate, alkyl boronate, —C(═NR¹⁴)—NR¹⁵R¹⁶, —NH—C(═NR¹⁴)NR¹⁵R¹⁶, —NH—C(═NR¹⁴)R¹⁴, an optionally substituted cycloaliphatic or non-aromatic heterocyclic group, or an optionally substituted aromatic group; R¹⁴ is —H, alkyl or an optionally substituted aromatic or aralkyl group; and R¹⁵ and R¹⁶ are independently —H, alkyl or an optionally substituted aromatic or aralkyl group; or —NR¹⁵R¹⁶ is an optionally substituted nitrogen-containing non-aromatic heterocyclic group.

X₁ is N, or CR⁴.

R⁴ is —H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl).

Each G₁ is independently —R^{13b}, -V₃-R¹³, -V₃-R^{13a}, -T₀-T₁-V₃-R¹³, -T₀-T₁-V₃-R^{13a}, -T₀-T₁-R^{13a}, -T₀-Cy-V₄-R¹³, -T₀-Cy-V₄-R^{13a}, -T₀-Cy-T₁-V₄-R¹³, -T₀-Cy-T₁-V₄-R^{13a}, T₀-Cy-R¹³, or -T₀-Cy-R^{13a}; or n is 2, one G₁ is (-T₂-R²⁰⁰)ₓ and the other G₁ is (-T₃-V₅-R⁵⁰)_{y}, x is 1 or 2, y is 0 or 1 and x+y is 1 or 2.

T₀ is absent, —CH₂—, —CH₂—CH₂—, or —CH₂—CH₂—CH₂—.

T₁ is —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂.

T₂ is a covalent bond, —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂—.

T₃ is a covalent bond, —O—, —NH—, —C(O)O—, —C(O)— or —C(O)NH—.

Cy is an optionally substituted arylene group or an optionally substituted non-aromatic heterocyclene or non-aromatic carbocyclene group.

V₃ is an optionally substituted C1-C8 alkylidene, provided that V₃ is a C2-C8 alkylidene when T₁ is —O—, —N(R⁶)—, —C(O)O—, or —C(O)N(R⁶)— and R¹³ is —CN, —OR¹², —NR¹¹R¹², —NR¹¹C(O)R¹², —OC(O)R¹², —NR¹¹C(O)NR¹¹R¹², —OC(O)NR¹¹R¹² or —NR¹¹C(O)OR¹², and wherein V₃ is optionally substituted with alkyl, halo, haloalkyl, alkoxy, hydroxy, NR¹¹R¹² or oxo.

V₄ is an optionally substituted bivalent C1-C8 aliphatic group provided that V₄ is a C2-C8 aliphatic group when T₁ is —O—, —N(R⁶)—, —C(O)O—, or —C(O)N(R⁶)— and R¹³ is —CN, —OR¹², —NR¹¹R¹², —NR¹¹C(O)R¹², —OC(O)R¹², —NR¹¹C(O)NR¹¹R¹², —OC(O)NR¹¹R¹² or —NR¹¹C(O)OR¹², and wherein V₄ is optionally substituted with alkyl, halo, haloalkyl, alkoxy, hydroxy, NR¹¹R¹² or oxo.

V₅ is a covalent bond or a C1-C4 alkylidene, provided that V₅ is C2-C4 alkylidene when T₃ is —O—, —NH—, —C(O)O—, or —C(O)NH— and R⁵⁰ is —CN, —OH, —NR⁵¹R⁵², —NHC(O)R⁵¹, —NHC(O)NR⁵¹R⁵², —NHC(O)OR⁵¹ or a substituted or unsubstituted nitrogen-containing non-aromatic heterocyclic group wherein a C1-C4 alkylidene group represented by V₅ is optionally substituted with a spirocyclopropyl group or one or two methyl groups and wherein a C1-C4 alkylidene group represented by V₅ is optionally fused to a cyclopropyl group.

Each R⁶ is independently —H or C1-C3 alkyl.

Each R¹¹ is independently —H or a C1-C3 alkyl group.

Each R¹² is independently —H or an optionally substituted alkyl, aromatic, aralkyl, non-aromatic heterocyclic or non-aromatic heterocyclylalkyl group; or —NR¹¹R¹² is an optionally substituted aromatic or non-aromatic nitrogen-containing heterocyclic group.

R¹³ is —OR¹², —CN, —COOR¹², —NR¹¹R¹², —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —O—C(O)—OR¹², —OC(O)—NR¹¹R¹², —NR¹¹CO—CH(OR¹⁸)—R¹², —NR¹¹CO—CH(NR¹⁸R¹⁸)—R¹², —NR¹¹CO—(CH₂)ₘCH(NR¹⁸R¹⁸)—R¹², —OC(O)—CH(OR¹⁸)—R¹², —OC(O)—CH(NR¹⁸R¹⁸)—R¹², —NR¹¹CO—C(R¹⁹R¹⁹)—OR¹², —NR¹¹CO—C(R¹⁹R¹⁹)—NR¹¹R¹², —OC(O)—C(R¹⁹R¹⁹)—OR¹², —OC(O)—C(R¹⁹R¹⁹)—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², —C(O)NR¹¹R¹², —NHC(O)NR¹¹R¹², or —C(═NR¹¹)—NR¹¹R¹².

R^{13a} is an optionally substituted nitrogen-containing heteroaromatic group or a nitrogen-containing non-aromatic heterocyclic group.

R^{13b} is an optionally substituted nitrogen-containing heteroaromatic group or a nitrogen-containing non-aromatic heterocyclic group.

Each R¹⁸ is independently —H, a C1-C3 alkyl group, —C(O)H, —C(O)—(C1-C3 alkyl), —C(O)NH₂, —C(O)NH-(C1-C3 alkyl), —C(O)N—(C1-C3 alkyl)₂, —C(O)O—(C1-C3 alkyl), —S(O)₂(C1-C3 alkyl) or —NR¹⁸R¹⁸ taken together is a substituted or unsubstituted non-aromatic nitrogen-containing heterocyclic group.

Each R¹⁹ is independently —H, a C1-C3 alkyl group or —C(R¹⁹R¹⁹)— taken together is a C3-C8 cycloalkyl group.

R⁵⁰ is —CN, —OR⁵¹, —NR⁵¹R⁵², —C(O)NR⁵¹R⁵², —NHC(O)R⁵¹, —NHC(O)NR⁵¹R⁵², —NHC(O)OR⁵¹, —C(O)OR⁵¹ or an optionally substituted aromatic group or non-aromatic heterocyclic group.

Each R⁵¹ and each R⁵² are independently —H or C1-C3 alkyl or —NR⁵¹R⁵² is an optionally substituted non-aromatic heterocyclic group.

R²⁰⁰ is an optionally substituted C2-C4 alkenyl or C2-C4 alkynyl group.

m is 1 or 2.

n is 1 or 2.

Another embodiment of the present invention is a method of treating cancer in a subject. The method comprises administering to the subject an effective amount of a Chk-1 inhibitor disclosed herein.

Yet another embodiment of the present invention is a method of inhibiting Chk-1 in a subject in need of such treatment. The method comprises administering to the subject an effective amount of a Chk-1 inhibitor disclosed herein.

Yet another embodiment of the present invention is a method of treating a proliferative disorder in a subject comprising administering an effective amount of a Chk-1 inhibitor disclosed herein.

Yet another embodiment of the present invention is a method of inhibiting Chk-1 in a cell in a subject in need of such treatment by contacting the cell with an effective amount of a Chk-1 inhibitor disclosed herein.

Yet another embodiment of the present invention is a method of inhibiting Chk-1 in a cell in vitro by contacting the cell with an effective amount of a Chk-1 inhibitor disclosed herein.

Yet another embodiment of the present invention is a pharmaceutical composition comprising a Chk-1 inhibitor disclosed herein and a pharmaceutically effective excipient, carrier or diluent. The pharmaceutical compositions can be used in therapy, e.g., to inhibit Chk-1 activity in a subject in need of such inhibition or to treat a subject with cancer.

Yet another embodiment of the present invention is the use of a Chk-1 inhibitor disclosed herein for the manufacture of a medicament for inhibiting Chk-1 in a subject in need of such inhibition or for treating a subject with cancer.

The compounds disclosed herein are effective inhibitors of Chk-1. They are therefore expected to be effective in treating subjects with cancer and enhancing the effectiveness of many current anti-cancer therapies, including radiation therapy and anti-cancer agents that exert their cytotoxic activity by damaging the genetic material of cancer cells and inhibiting cellular replication. In addition, the disclosed Chk-1 inhibitors, when used in combination with current anti-cancer therapies are expected to be effective against multidrug resistant cancers.

### DETAILED DESCRIPTION OF THE INVENTION

The present invention is directed to Chk-1 inhibitors represented by Structural Formula (I) and to novel methods of therapy utilizing the Chk-1 inhibitors represented by Structural Formula (I):

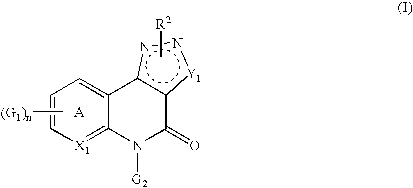

The values and preferred values for the values for the variables in Structural Formula (I) are as described below.

Ring A is substituted with one or two G₁. Additionally, Ring A is optionally substituted at any one or more substitutable ring carbon atoms. Suitable Ring A substituents include those described below in the section describing suitable aryl group substituents generally. Preferred substitutents are represented by R⁵ and are independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl). Preferably, each R⁵ is independently —H, halogen, C1-C3 alkyl, C1-C3 haloalkyl, C1-C3 alkoxy, or C1-C3 haloalkoxy. More preferably, each R⁵ is independently —H, halogen, —CH₃, halomethyl, —OCH₃, or haloalkoxy;

Y₁ is N or CR³. Preferably, Y₁ is CR³.

X₁ is N, or CR⁴. Preferably, X₁ is CR⁴.

R² is —H or a group that is cleavable in vivo. Preferably R² is —H.

R³ is —H, halogen, alkyl, haloalkyl or -V₁-R⁷, wherein V₁ is a covalent bond or a C1-C4 alkylidene optionally substituted with one or more —OR¹⁴, —NR¹⁵R¹⁶, alkyl, hydroxyalkyl, alkoxyalkyl, aminoalkyl, or with a spiro cycloalkyl group; R⁷ is —OR¹⁴, —SR¹⁴, —CONR¹⁵R¹⁶, —NR¹⁵R¹⁶, —NHC(O)NR¹⁵R¹⁶, —CN, —COOR¹⁴, —NHC(O)H, —NHC(O)R¹⁴, —OC(O)R¹⁴, —OC(O)NR¹⁵R¹⁶, —NHC(O)—OR¹⁴, —S(O)₂NR¹⁵R¹⁶, —S(O)₂(R¹⁴), boronate, alkyl boronate, —C(═NR¹⁴)—NR¹⁵R¹⁶, —NH—C(═NR¹⁴)NR¹⁵R¹⁶, —NH—C(═NR¹⁴)R¹⁴, an optionally substituted cycloaliphatic or non-aromatic heterocyclic group, or an optionally substituted aromatic group (or carbocyclic or heteroaromatic group). Preferably, R³ is —H, methyl, ethyl, n-propyl, iso-propyl, C1-C3 haloalkyl, or V₁-R⁷, wherein V₁ is a covalent bond or a C1-C2 alkylidene optionally substituted with one or two methyl groups or with a spiro cyclopropyl group; R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl; additional values for R³ include —OC(O)N(CH₃)₂, —NHC(O)NH₂, —NHC(O)NH(CH₃), —NHC(O)N(CH₃)₂, —NHC(O)OCH₃. More preferably, R³ is methyl or ethyl; or R³ is V₁-R⁷, wherein V₁ is a C1-C2 alkylidene and R⁷ is —OH or —OCH₃; or V₁ is a covalent bond and R⁷ is cyclopropyl, cyclopentyl, furyl or tetrahydrofuryl.

R⁴ is —H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl). Preferably, R⁴ is —H, C1-C3 alkyl, C1-C3 haloalkyl, halogen, hydroxy, C1-C3 alkoxy, C1-C3 haloalkoxy, —NH₂, C1-C3 alkylamino, C1-C3 dialkylamino, —NHC(O)H, —NHC(O)(C1-C3 alkyl), —C(O)NH₂, —C(O)NH(C1-C3 alkyl) or —C(O)N(C1-C3 alkyl)₂. More preferably, R⁴ is —H, halogen, C1-C3 alkyl, C1-C3 haloalkyl, C1-C3 alkoxy, or C1-C3 haloalkoxy. Alternatively, R⁴ is —H, halogen, —CH₃, halomethyl, —OCH₃, or haloalkoxy.

Each G₁ is independently —R^{13b}, -V₃-R¹³, -V₃-R^{13a}, -T₀-T₁-V₃-R¹³, -T₀-T₁-V₃-R^{13a}, -T₀-T₁-R^{13a}, -T₀-Cy-V₄-R¹³, -T₀-Cy-V₄-R^{13a}, -T₀-Cy-T₁-V₄-R¹³, -T₀-Cy-T₁-V₄-R^{13a}, T₀-Cy-R¹³, or -T₀-Cy-R^{13a}. Additional values for G₁ include (-T₂-R²⁰⁰)ₓ and (-T₃-V₅-R⁵⁰)_{y}, where x is 1 or 2, y is 0 or 1 and x+y is 1 or 2.

In one preferred embodiment, G₁ is —R^{13b}. Alternatively, G₁ is -V₃-R¹³, -V₃-R^{13a}, -T₀-T₁-V₃-R¹³, -T₀-T₁-V₃-R^{13a}, or -T₀-T₁-R¹³a. In another alternative, G₁ is -T₀-Cy-V₄-R¹³, -T₀-Cy-V₄-R^{13a}, -T₀-Cy-T₁-V₄-R¹³, -T₀-Cy-T₁-V₄-R^{13a}, T₀-Cy-R¹³, or -T₀-Cy-R^{13a}.

G₂ is —H, or a C1-C8 aliphatic group optionally substituted with one or more fluoro, —OR¹², —CONR¹¹R¹², —COOR¹², cycloalkyl or phenyl, wherein the cycloalkyl and phenyl are optionally substituted with halo or alkyl. Preferably, G₂ is —H, or a C1-C6 aliphatic group optionally substituted with one or more, fluoro, —OR¹², —CONR¹¹R¹², —COOR¹², cycloalkyl or phenyl, wherein the cycloalkyl and phenyl are optionally substituted with halo or alkyl. Preferably, the cycloalkyl and phenyl substituents on G₂ are unsubstituted. More preferably, G₂ is C1-C4 alkyl, wherein the alkyl is optionally substituted with fluoro or G₂ is a C3-C8 cycloalkyl wherein the cycloalkyl is optionally substituted with halo or alkyl.

In an alternative embodiment, G2 is -W₁-R¹—.

R¹ is —H, —CONR¹¹R¹², —COOR¹², fluoro, or a cycloalkyl wherein the cycloalkyl is optionally substituted with halo or alkyl and W₁ is a linear C1-C6 alkylidene chain; or R¹ is —OR¹² and W₁ is a linear C2-C6 alkylidene group, wherein the alkylidene group represented by W₁ is optionally substituted with one or more —CH₃ or fluoro groups; or -W₁-R¹ is —H. More preferably, W₁ is a linear C1-C4 alkylidene chain optionally substituted with one or more —CH₃ or fluoro groups and R¹ is —H, fluoro or a cycloalkyl wherein the cycloalkyl is optionally substituted with halo or alkyl.

T₀ is absent, —CH₂—, —CH₂—CH₂—, or —CH₂—CH₂—CH₂—. Preferably T₀ is absent.

T₁ is —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂. Preferably T₁ is —O— or —N(R⁶);

T₂ is a covalent bond, —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂—. Preferably T₂ is a covalent bond, —S(O), —SO₂—, —C(O)—, —OC(O)—, —N(R⁶)C(O)—, or —N(R⁶)SO₂. More preferably, T₂ is a covalent bond.

T₃ is a covalent bond, —O—, —NH—, —C(O)O—, —C(O)— or —C(O)NH—. Preferably, T₃ is a covalent bond —O— or —N(R⁶). More preferably, T₃ is a covalent bond.

T₁₁ is —S(O)—, —S(O)₂—, —C(O)—, —C(O)O—, —C(O)N(R⁶)—, or —SO₂N(R⁶)—. Preferably, T₁₁ is —C(O)—, —C(O)N(R⁶)—, or —SO₂N(R⁶)—.

Cy is an optionally substituted arylene group or an optionally substituted non-aromatic heterocyclene or non-aromatic carbocyclene group. Preferably, Cy is an optionally substituted phenylene, pyrrolylene, thienylene, furanylene, imidazolylene, triazolylene, tetrazolylene oxazolylene, isoxazolylene, oxadiazolylene, pyrazolylene, pyridinylene, pyrimidylene, pyrazinylene, thiazolylene, cyclopropylene, cyclopentylene, cyclohexylene, cycloheptylene, piperidinylene, piperazinylene, pyrrolidinylene, pyrazolidinylene, imidazolidinylene, tetrahydrofuranylene, tetrahydrothienylene, isooxazolidinylene, oxazolidinylene, isothiazolidinylene, thiazolidinylene, oxathiolanylene, dioxolanylene, or dithiolanylene. More preferably, Cy is [2,5]thienylene or [2,5]furanylene. Suitable substituents for an arylene Cy group include those described below in the section describing aromatic group substituents generally; and suitable substituents for non-aromatic hetercyclene and carbocyclene Cy groups include those described below in the sections describing suitable substituents for a non-aromatic heterocyclic group and aliphatic groups generally. Preferred substituents for a substitutable aromatic ring carbon in a group represented by Cy and a substitutable ring carbon or ring nitrogen atom in a non-aromatic ring represented by Cy are as described below for R^{13a} and R^{13b}.

V₃ is an optionally substituted C1-C8 alkylidene, provided that V₃ is a C2-C8 alkylidene when T₁ is —O—, —N(R⁶)—, —C(O)O—, or —C(O)N(R⁶)— and R¹³is —CN, —OR¹², —NR¹¹R¹², —NR¹¹C(O)R¹², —OC(O)R¹², —NR¹¹C(O)NR¹¹R¹², —OC(O)NR¹¹R¹² or —NR¹¹C(O)OR¹², and wherein V₃ is optionally substituted with alkyl, halo, haloalkyl, alkoxy, hydroxy, NR¹¹R¹² or oxo. Preferably, V₃ is C1-C4 alkylidene. More preferably, V₃ is C1-C2 alkylidene.

V₄ is an optionally substituted bivalent C1-C8 aliphatic group provided that V₄ is a C2-C8 aliphatic group when T₁ is —O—, —N(R⁶)—, —C(O)O—, or —C(O)N(R⁶)— and R¹³ is —CN, —OR¹², —NR¹¹R¹², —NR¹¹C(O)R¹², —OC(O)R¹², —NR¹¹C(O)NR¹¹R¹², —OC(O)NR¹¹R¹² or —NR¹¹C(O)OR¹², and wherein V₄ is optionally substituted with alkyl, halo, haloalkyl, alkoxy, hydroxy, NR¹¹R¹² or oxo. Preferably, V₄ is C1-C4 alkylidene, alkenylidene or alkynylidene group optionally substituted with C1-C3 alkyl. More preferably, V₄ is C1-C4 alkylidene. Even more preferably, V₄ is C1-C2 alkylidene.

V₅ is a covalent bond or a C1-C4 alkylidene, provided that V₅ is C2-C4 alkylidene when T₃ is —O—, —NH—, —C(O)O—, or —C(O)NH— and R⁵⁰ is —CN, —OH, —NR⁵¹R⁵², —NHC(O)R⁵¹, —OC(O)R⁵¹, —NHC(O)NR⁵¹R⁵², —OC(O)NR⁵¹R⁵², —NHC(O)OR⁵¹ or a substituted or unsubstituted nitrogen-containing non-aromatic heterocyclic group. The C1-C4 alkylidene group represented by V₅ is optionally substituted with a spirocyclopropyl group or one or two methyl groups and the C1-C4 alkylidene group represented by V₅ is optionally fused to a cyclopropyl group.

V₆ is a C1-C4 alkylidene, wherein V₆ is optionally substituted with alkyl, halo, haloalkyl, alkoxy, hydroxy, NR¹¹R¹² or oxo. Preferably, V₆ is a C1-C4 alkylidene group optionally substituted with C1-C3 alkyl. More preferably, V₆ is C1-C4 alkylidene. Even more preferably, V₆ is C1-C2 alkylidene.

Each R⁶ is independently —H or C1-C3 alkyl.

Each R¹¹ is independently —H or a C1-C3 alkyl group.

Each R¹² is independently —H, or an optionally substituted alkyl, aryl, aralkyl, non-aromatic heterocyclic or non-aromatic heterocyclylalkyl group. Alternatively, R¹² is —H, an optionally substituted alkyl, or an optionally substituted non-aromatic heterocyclic group. Preferably, R¹² is H, an optionally substituted alkyl, imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl, thiadiazolyl, pyrrolidinyl, piperidinyl, morpholinyl, pyrazinyl, thiomorpholinyl, pyrrolidinyl, piperidinyl, pyranzinyl, thiomorpholinyl, tetrahydroquinolinyl or tetrahydroisoquinolinyl. More preferably R¹² is —H, an optionally substituted alkyl or an optionally substituted piperidinyl ring;

NR¹¹R¹² is an optionally substituted aromatic or non-aromatic nitrogen-containing heterocyclic group. Preferably, —NR¹¹R¹² is imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl, thiadiazolyl, pyrrolidinyl, piperidinyl, morpholinyl, pyrazinyl, thiomorpholinyl, pyrrolidinyl, piperidinyl, pyranzinyl, thiomorpholinyl, tetrahydroquinolinyl or tetrahydroisoquinolinyl. More preferably —NR¹¹R¹² is pyrrolidinyl, piperidinyl, piperazinyl, tetrahydroisoquinolinyl, morpholinyl or pyrazolyl.

R¹³ is —OR¹², —CN, —COOR¹², —NR¹¹R¹², —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —O—C(O)—OR¹², —OC(O)—NR¹¹R¹², —NR¹¹CO—CH(OR¹⁸)—R¹², —NR¹¹CO—CH(NR¹⁸R¹⁸)—R¹², —NR¹¹CO—(CH₂)ₘCH(NR¹⁸R¹⁸)—R¹², —OC(O)—CH(OR¹⁸)—R¹², —OC(O)—CH(NR¹⁸R¹⁸)—R¹², —NR¹¹CO—C(R¹⁹R¹⁹)—OR¹², —NR¹¹CO—C(R¹⁹R¹⁹)—NR¹¹R¹², —OC(O)—C(R¹⁹R¹⁹)—OR¹², —OC(O)—C(R¹⁹R¹⁹)—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², —C(O)NR¹¹R¹², —NHC(O)NR¹¹R¹², or —C(═NR¹¹)—NR¹¹R¹².

In one prefered embodiment, R¹³ is —CN, —OR¹², —NR¹¹R¹², —NHC(O)R¹², —NHC(O)OR¹², —NHC(O)NR¹¹R¹², —NHC(O)OR¹², or —OC(O)R¹². Preferably, R¹³ is —NR¹¹R¹².

In another preferred embodiment, R¹³ is —OH, —CN, C1-C3 alkoxy, NH₂, C1-C3 alkylamino, C1-C3 dialkylamino, C1-C3 hydroxyalkyl, or C1-C3 haloalkylamino. Preferably, R¹³ is NH₂, C1-C3 alkylamino, or C1-C3 dialkylamino.

In yet another preferred embodiment, R¹³ is —OH, —CN, C1-C3 alkoxy, or NR¹¹R¹², where R¹¹ is —H or a C1-C3 alkyl group and R¹² is —H, an optionally substituted alkyl, or an optionally substituted non-aromatic heterocyclic group, or NR¹¹R¹² is an optionally substituted aromatic or non-aromatic nitrogen containing heterocyclic group. Preferably, R¹³ is NH₂, C1-C3 alkylamino, or C1-C3 dialkylamino. More preferably, R¹³ is —NH₂, —NHCH₃, —N(CH₃)₂, —NH(CH₂CH₃), or —N(CH₂CH₃)₂.

R^{13a} and R^{13b} are independently an optionally substituted nitrogen-containing heteroaromatic group or a nitrogen-containing non-aromatic heterocyclic group. Suitable substituents for a nitrogen-containing heteroaromatic group or nitrogen-containing non-aromatic heterocyclic group represented by R^{13a} or R^{13b} include those described below for nitrogen-containing heteroaromatic groups generally and nitrogen-containing non-aromatic heterocyclic groups generally. Preferably, each substitutable ring nitrogen atom of the group represented by R^{13a} or R^{13b} is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group; each substitutable ring carbon atom of a non-aromatic ring in the group represented by R^{13a} or R^{13b} is optionally substituted with a C1-C3 alkyl group, hydroxy, fluoro, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 fluoroalkylamido, amino (C1-C3) alkyl, (C1-C3)alkylamino(C1-C3)alkyl, (C1-C3)dialkylamino(C1-C3)alkyl, hydroxy(C1-C3)alkyl, (C1-C3)alkoxy(C1-C3)alkyl; each substitutable ring carbon atom of an aromatic ring in the group represented by R^{13a} or R^{13b} is optionally substituted with halo, hydroxy, cyano, C1-C3 alkyl, C1-C3 fluoroalkyl, C1-C3 alkoxy, C1-C3 fluoroalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, —NR′C(O)O—(C1-C3 alkyl)-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl); and each R′ is hydrogen or a C1-C3 alkyl group.

In preferred embodiment, R^{13a} and R^{13b} are independently an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl, and azabicyclopentyl, azabicyclohexyl, azabicycloheptyl, azabicyclooctyl, azabicyclononyl, azabicyclodecyl, diazabicyclohexyl, diazabicycloheptyl, diazabicyclooctyl, diazabicyclononyl, or diazabicyclodecyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl, and thiadiazolyl. More preferably, R^{13a} and R^{13b} are independently an optionally substituted non-aromatic heterocyclic group selected from N-pyrrolidinyl, N-piperidinyl, N-morpholinyl, N-piperazinyl, N-thiomorpholinyl, N-azetidinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl and 3-oxo-N-8-azabicyclo[3.2.1]octyl or N-8-azabicyclo[3.2.1]octyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl and thiadiazolyl.

In one embodiment, R^{13a} is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl, 3-oxo-N-8-azabicyclo[3.2.1]octyl or N-8-azabicyclo[3.2.1]octyl. In certain preferred embodiments, R^{13a} is N-pyrrolidinyl, 2,5-dimethyl-N-pyrrolidinyl, N-piperidinyl, N′-methyl-N-piperazinyl, N-tetrahydroisoquinolinyl, N-morpholinyl, 3-oxo-N-8-azabicyclo[3.2.1]octyl or N-8-azabicyclo[3.2.1]octyl.

In one embodiment, R^{13b} is an optionally substituted nitrogen-containing heteroaromatic group. More preferably, R^{13b} is an optionally substituted imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl or thiadiazolyl. More preferably R^{13b} is pyrazolyl or N-methyl-pyrazolyl.

R¹⁴ is —H, alkyl or an optionally substituted aromatic or aralkyl group; and R¹⁵ and R¹⁶ are independently —H, alkyl or an optionally substituted aromatic or aralkyl group; or —NR¹⁵R¹⁶ is an optionally substituted nitrogen-containing non-aromatic heterocyclic group. Preferably R¹⁴ is —H or alkyl. Preferably, —NR¹⁵R¹⁶ is pyrrolidinyl, piperidinyl, morpholinyl, pyrazinyl, thiomorpholinyl, pyrazinyl, thiomorpholinyl, tetrahydroquinolinyl or tetrahydroisoquinolinyl.

Each R¹⁸ is independently —H, a C1-C3 alkyl group, —C(O)H, —C(O)—(C1-C3 alkyl), —C(O)NH₂, —C(O)NH-(C1-C3 alkyl), —C(O)N—(C1-C3 alkyl)₂, —C(O)O—(C1-C3 alkyl), —S(O)₂(C1-C3 alkyl) or —NR¹⁸R¹⁸ taken together is a substituted or unsubstituted non-aromatic nitrogen-containing heterocyclic group. Preferably, each R¹⁸ is independently —H, a C1-C3 alkyl group, and —NR¹⁸R¹⁸ is pyrrolidinyl, piperidinyl, morpholinyl, pyrazinyl, thiomorpholinyl, pyrazinyl, thiomorpholinyl, tetrahydroquinolinyl or tetrahydroisoquinolinyl.

Each R¹⁹ is independently —H, a C1-C3 alkyl group or —C(R¹⁹R¹⁹)— taken together is a C3-C8 cycloalkyl group.

Each R²⁰ is independently —H or C1-C3 alkyl.

R⁵⁰ is —CN, —OR⁵¹, —NR⁵¹R⁵², —C(O)NR⁵¹R⁵², —NHC(O)R⁵¹, —NHC(O)NR⁵¹R⁵², —NHC(O)OR⁵¹, —C(O)OR⁵¹ or an optionally substituted aromatic group or non-aromatic heterocyclic group. Preferably, R⁵⁰ is —CN, —OR⁵¹, —NR⁵¹R⁵², —C(O)NR⁵¹R⁵², —NHC(O)R⁵¹, —NHC(O)NR⁵¹R⁵², —NHC(O)OR⁵¹, —C(O)OR⁵¹ or an optionally substituted aromatic group or non-aromatic heterocyclic group. More preferably, R⁵⁰ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen. Even more preferably, R⁵⁰ is —OH, —CN, C1-C3 alkoxy, NH₂, C1-C3 alkylamino, C1-C3 dialkylamino, C1-C3 hydroxyalkyl, or C1-C3 haloalkylamino. Preferably, R⁵⁰ is NH₂, C1-C3 alkylamino, or C1-C3 dialkylamino. Suitable substitutents for the nitrogen-containing heteroaromatic group or nitrogen-containing non-aromatic heterocyclic group represented by R⁵⁰ are as described below for nitrogen-containing heteroaromatic groups generally and nitrogen-containing non-aromatic heterocyclic groups generally.

Each R⁵¹ and each R⁵² are independently —H or C1-C3 alkyl or —NR⁵¹R⁵² is an optionally substituted non-aromatic heterocyclic group. Preferably, each R⁵¹ or R⁵² is independently —H, a C1-C3 alkyl group, and, —NR⁵¹R⁵² is pyrrolidinyl, piperidinyl, morpholinyl, pyrazinyl, thiomorpholinyl, pyrrolidinyl, piperidinyl, pyranzinyl, thiomorpholinyl, tetrahydroquinolinyl or tetrahydroisoquinolinyl.

R⁶⁰ is —OR¹², —CN, —COOR¹², —NR¹¹R¹², —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —O—C(O)—OR¹², —OC(O)—NR¹¹R¹², —NR¹¹CO—CH(OR⁶²)—R¹², —NR¹¹CO—CH(NR⁶²R⁶²)—R¹², —NR¹¹CO—(CH₂)_{z}CH(NR⁶²R⁶²)—R¹², —OC(O)—CH(OR⁶²)—R¹², —OC(O)—CH(NR⁶²R⁶²)—R¹², —NR¹¹CO—C(R⁶²R⁶³)—OR¹², —NR¹¹CO—C(R⁶³R⁶³)—NR¹¹R¹², —OC(O)—C(R⁶³R⁶³)—OR¹², —OC(O)—C(R⁶³R⁶³)—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², —C(O)NR¹¹R¹², —NHC(O)NR¹¹R¹², or —C(═NR¹¹)—NR¹¹R¹².

In one preferred embodiment, R⁶⁰ is —CN, —OR¹², —NR¹¹R¹², —NHC(O)R¹², —NHC(O)OR¹², —NHC(O)NR¹¹R¹², —NHC(O)OR¹², or —OC(O)R¹².

In another preferred embodiment, R⁶⁰ is —OH, —CN, C1-C3 alkoxy, NH₂, C1-C3 alkylamino, C1-C3 dialkylamino, C1-C3 hydroxyalkyl, or C1-C3 haloalkylamino. Preferably, R⁶⁰ is NH₂, C1-C3 alkylamino, or C1-C3 dialkylamino.

In yet another preferred embodiment, R⁶⁰ is —OH, —CN, C1-C3 alkoxy, or NR¹¹R¹², where R¹¹ is —H or a C1-C3 alkyl group and R¹² is —H, an optionally substituted alkyl, or an optionally substituted non-aromatic heterocyclic group, or NR¹¹R¹² is an optionally substituted aromatic or non-aromatic nitrogen containing heterocyclic group. Preferably, R⁶⁰ is NH₂, C1-C3 alkylamino, or C1-C3 dialkylamino. More preferably, R⁶⁰ is —NH₂, —NHCH₃, —N(CH₃)₂, —NH(CH₂CH₃), or —N(CH₂CH₃)₂.

R⁶¹ is an optionally substituted nitrogen-containing heteroaromatic group or a nitrogen-containing non-aromatic heterocyclic group. Suitable substituents for a nitrogen-containing non-aromatic heteraromatic group or nitrogen-containing non-aromatic heterocyclic group represented by R⁶⁰ are as described below for nitrogen containing heteroaromatic groups generally and nitrogen-containing non-aromatic heterocyclic groups generally. Preferably, each substitutable ring nitrogen atom of the group represented by R⁶¹ is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group; each substitutable ring carbon atom of a non-aromatic ring in the group represented by R⁶¹ is optionally substituted with a C1-C3 alkyl group, hydroxy, fluoro, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 fluoroalkylamido, amino (C1-C3) alkyl, (C1-C3)alkylamino(C1-C3)alkyl, (C1-C3)dialkylamino(C1-C3)alkyl, hydroxy(C1-C3)alkyl, (C1-C3)alkoxy(C1-C3)alkyl; each substitutable ring carbon atom of an aromatic ring in the group represented by R⁶¹ is optionally substituted with halo, hydroxy, cyano, C1-C3 alkyl, C1-C3 fluoroalkyl, C1-C3 alkoxy, C1-C3 fluoroalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, —NR′C(O)O—(C1-C3 alkyl)-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl); and each R′ is hydrogen or a C1-C3 alkyl group.

In a preferred embodiment, R⁶¹ is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl and azabicyclopentanyl, azabicyclohexanyl, azabicycloheptanyl, azabicyclononanyl, azabicyclodecanyl, diazabicyclohexanyl, diazabicycloheptanyl, diazabicyclooctanyl, diazabicyclononanyl, or diazabicyclodecanyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl and thiadiazolyl.

In a preferred embodiment, R⁶¹ is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, and tetrahydroisoquinolinyl, or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl, thiadiazolyl, azabicyclopentanyl, azabicyclohexanyl, azabicycloheptanyl, azabicyclononanyl, azabicyclodecanyl, diazabicyclohexanyl, diazabicycloheptanyl, diazabicyclooctanyl, diazabicyclononanyl, or diazabicyclodecanyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl and thiadiazolyl. More preferably, R⁶¹ is an optionally substituted non-aromatic heterocyclic group selected from N-pyrrolidinyl, N-piperidinyl, N-morpholinyl, N-piperazinyl, N-thiomorpholinyl, N-azetidinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl and N-tetrahydroisoquinolinyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl, thiadiazolyl, 3-oxo-N-8-azabicyclo[3.2.1]octyl or N-8-azabicyclo[3.2.1]octyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl and thiadiazolyl.

In one preferred embodiment, R⁶¹ is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl, 3-oxo-N-8-azabicyclo[3.2.1]octyl or N-8-azabicyclo[3.2.1]octyl. In a certain preferred embodiment, R⁶¹ is N-pyrrolidinyl, 2,5-dimethyl-N-pyrrolidinyl, N-piperidinyl, N′-methyl-N-piperazinyl, N-tetrahydroisoquinolinyl, N-morpholinyl 3-oxo-N-8-azabicyclo[3.2.1]octyl or N-8-azabicyclo[3.2.1]octyl.

Each R⁶² is independently —H, a C1-C3 alkyl group, —C(O)H, —C(O)—(C1-C3 alkyl), —C(O)NH₂, —C(O)NH—(C1-C3 alkyl), —C(O)N—(C1-C3 alkyl)₂, —C(O)O—(C1-C3 alkyl), —S(O)₂(C1-C3 alkyl) or —NR⁶²R⁶² taken together is a substituted or unsubstituted non-aromatic nitrogen-containing heterocyclic group. Preferably, each R⁶² is independently —H, a C1-C3 alkyl group, and, —NR⁶²R⁶² is pyrrolidinyl, piperidinyl, morpholinyl, pyrazinyl, thiomorpholinyl, pyrrolidinyl, piperidinyl, pyranzinyl, thiomorpholinyl, tetrahydroquinolinyl or tetrahydroisoquinolinyl.

Each R⁶³ is independently —H, a C1-C3 alkyl group or —C(R⁶³R⁶³)— taken together is a C3-C8 cycloalkyl group.

R²⁰⁰ is an optionally substituted C2-C4 alkenyl or C2-C4 alkynyl group. In one embodiment, R²⁰⁰ is —C≡CR²⁰¹, —CH═CHR²⁰¹, —C≡C—(C(R²⁰R²⁰))ₚR²⁰², or —CH═CH—(C(R²⁰R²⁰))R²⁰². Preferably R²⁰⁰ is —C≡C—(C(R²⁰R²⁰))ₚR²⁰² or —C═C—(C(R²⁰R²⁰))ₚR²⁰². More preferably R²⁰⁰ is —C≡C—(C(R²⁰R²⁰))ₚR²⁰². In another embodiment, R²⁰⁰ is —C≡C—R²⁰³ or —C═CHR²⁰³.

R²⁰¹ is —H, alkyl, haloalkyl, hydroxyalkyl, CO₂R⁵¹, or an optionally substituted aromatic group or non-aromatic heterocyclic group. Preferably, R²⁰¹ is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-azetidinyl 3-pyrrolidinyl, 3-piperidinyl, 3-morpholinyl, 3-thiomorpholinyl, 3-azetidinyl, 4-piperidinyl, tetrahydroquinolinyl, tetrahydroisoquinolinyl, N-tetrahydroquinolinyl, N-tetrahydroisoquinolinyl 3-oxo-N-8-azabicyclo[3.2.1]octyl or N-8-azabicyclo[3.2.1]octyl.

R²⁰² is —H, —CN, —OR⁵¹, —OC(O)NR⁵¹R⁵², —OC(O)R⁵¹, —NR⁵¹R⁵², —C(O)NR⁵¹R⁵², —NR⁵¹C(O)R⁵¹, —NR⁵¹C(O)NR⁵¹R⁵², —NR⁵¹C(O)OR⁵¹, —NR⁵¹S(O)₂R^{x}, —S(O)₂NR⁵¹, —CO₂R⁵¹ or an optionally substituted aromatic group or non-aromatic heterocyclic group. Preferably, R²⁰² is —CN, —OH, C1-C3 alkoxy, —NH₂, C1-C3 alkylamino, C1-C3 dialkylamino, an optionally substituted non-aromatic heterocyclic group selected from N-pyrrolidinyl, N-piperidinyl, N-morpholinyl, N-piperazinyl, N-thiomorpholinyl, 2-pyrrolidinyl, 2-piperidinyl, 2-piperazinyl, 2-morpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl, 3-oxo-N-8-azabicyclo[3.2.1]octyl or N-8-azabicyclo[3.2.1]octyl. More preferably, R²⁰² is NH₂, C1-C3 alkylamino, C1-C3 dialkylamino, an optionally substituted non-aromatic heterocyclic group selected from N-pyrrolidinyl, N-piperidinyl, N-piperazinyl, N-morpholinyl, N-azetidinyl, N-thiomorpholinyl, 2-pyrrolidinyl, 2-piperidinyl, 2-piperazinyl, 2-morpholinyl, 2-azetidinyl, 3-pyrrolidinyl, 3-piperidinyl, 3-morpholinyl, 3-thiomorpholinyl, 3-azetidinyl, 4-piperidinyl, tetrahydroquinolinyl, tetrahydroisoquinolinyl, N-tetrahydroquinolinyl and N-tetrahydroisoquinolinyl, 3-oxo-N-8-azabicyclo[3.2.1]octyl or N-8-azabicyclo[3.2.1]octyl.

Suitable substitutents for the nitrogen-containing heteroaromatic group or nitrogen-containing non-aromatic heterocyclic group represented by R²⁰¹ and R²⁰² are as described below for nitrogen-containing heteroaromatic groups generally and nitrogen-containing non-aromatic heterocyclic groups generally. Preferably, each substitutable ring nitrogen atom in an aromatic or non-aromatic heterocyclic group represented by R²⁰¹ or R²⁰² is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group; each substitutable ring carbon atom of a non-aromatic heterocyclic group represented by R²⁰¹ or R²⁰² is optionally substituted with a C1-C3 alkyl group, hydroxy, halo, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 haloalkylamido, (C1-C3)aminoalkyl, (C1-C3)alkoxyalkyl, (C1-C3)hydroxyalkyl; and each substitutable ring carbon atom of an aromatic group represented by R²⁰¹ or R²⁰² is optionally substituted with halo, hydroxy, cyano, C1-C3 alkyl, C1-C3 haloalkyl, C1-C3 alkoxy, C1-C3 haloalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, or —NR′C(O)O—(C1-C3 alkyl).-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl).

R²⁰³ has the formula -V₆-R⁶⁰, -V₆-R⁶¹, -T₁₁-V₆-R⁶⁰, or -T₁₁-V₆-R⁶¹.

R^{x} is alkyl or an optionally substituted aromatic group or non-aromatic heterocyclic group.

p is 1 or 2.

m is 1 or 2.

n is 1 or 2. Preferably n is 1.

z is an integer from 1 to 4.

In a preferred embodiment, the Chk-1 inhibitor of the present invention is represented by Structural Formula (II): 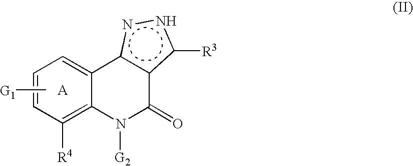
Values and preferred values for the variables in Structural Formula (II) are as described above for Structural Formula (I).

In another preferred embodiment the Chk-1 inhibitor of the present invention is represented by a Structural Formula selected from (III) and (IV):

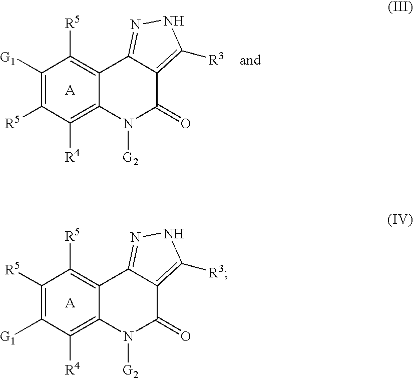

Each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl). The values and preferred values for the remaining variables in Structural Formulas (III) and (IV) are as described above for Structural Formula (I).

In another preferred embodiment the Chk-1 inhibitor of the present invention is represented by Structural Formula (V):

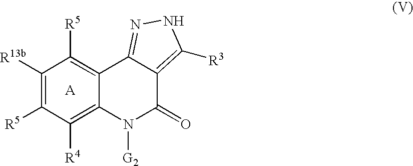

R⁵ is as defined above for Structural Formula (III) and (IV). The values and preferred values for each remaining variable in Structural Formula (V) are as described above for Structural Formula (I).

In a first more preferred embodiment, the Chk-1 inhibitor of the present invention is represented by Structural Formula (II), (III), (IV) or (V);

G₂ is C1-C4 alkyl optionally substituted with fluoro or C3-C8 cycloalkyl, wherein the cycloalkyl group is optionally substituted with halo or alkyl; and

the values for all other variables and their preferred values are as described above for Structural Formula (I).

In a second more preferred embodiment, the Chk-1 inhibitor of the present invention is represented by Structural Formula (III), (IV) or (V);

G₂ is C1-C4 alkyl optionally substituted with fluoro or C3-C8 cycloalkyl, wherein the cycloalkyl group is optionally substituted with halo or alkyl;

each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl);

the values for all other variables and their preferred values are as described above for Structural Formula (I).

In a third more preferred embodiment, the Chk-1 inhibitor of the present invention is represented by Structural Formula (III), (IV) or (V);

G₂ is C1-C4 alkyl optionally substituted with fluoro or C3-C8 cycloalkyl, wherein the cycloalkyl group is optionally substituted with halo or alkyl;

R³ is —H, methyl, ethyl, n-propyl, iso-propyl, C1-C3 haloalkyl, or V₁-R⁷, wherein V₁ is a covalent bond or a C1-C2 alkylidene optionally substituted with one or two methyl groups or with a spiro cyclopropyl group; and R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl;

each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl); and

the values for all other variables and their preferred values are as described above for Structural Formula (I).

In a fourth more preferred embodiment, the Chk-1 inhibitor of the present invention is represented by Structural Formula (III), (IV) or (V);

G₂ is C1-C4 alkyl optionally substituted with fluoro or C3-C8 cycloalkyl, wherein the cycloalkyl group is optionally substituted with halo or alkyl;

R³ is methyl, or ethyl; or R³ is V₁-R⁷, wherein V₁ is a C1-C2 alkylidene and R⁷ is —OH, —OCH₃; or V₁ is a covalent bond and R⁷ is cyclopropyl, cyclopentyl, furyl or tetrahydrofuryl;

each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl); and

the values for all other variables and their preferred values are as described above for Structural Formula (I).

In a fifth more preferred embodiment, the Chk-1 inhibitor of the present invention is represented by Structural Formula (III), (IV) or (V);

G₂ is C1-C4 alkyl optionally substituted with fluoro or C3-C8 cycloalkyl, wherein the cycloalkyl group is optionally substituted with halo or alkyl;

R³ is methyl, or ethyl; or R³ is V₁-R⁷, wherein V₁ is a C1-C2 alkylidene and R⁷ is —OH, —OCH₃; or V₁ is a covalent bond and R⁷ is cyclopropyl, cyclopentyl, furyl or tetrahydrofuryl;

R⁴ and each R⁵ are independently —H, halogen, C1-C3 alkyl, C1-C3 haloalkyl, C1-C3 alkoxy, or C1-C3 haloalkoxy; and

the values for all other variables and their preferred values are as described above for Structural Formula (I).

In a sixth more preferred embodiment, the Chk-1 inhibitor of the present invention is represented by Structural Formula (I), (II), (III) or (IV);

G₂ is C1-C4 alkyl optionally substituted with fluoro or C3-C8 cycloalkyl, wherein the cycloalkyl group is optionally substituted with halo or alkyl;

R¹³ is —CN, —OR¹², —NR¹¹R¹², —NHC(O)R¹², —NHC(O)OR¹², —NHC(O)NR¹¹R¹², —NHC(O)OR¹², or —OC(O)R¹²;

G₁ is -V₃-R¹³, -V₃-R^{13a}, -T₀-T₁-V₃-R¹³, -T₀-T₁-V₃-R^{13a}, or -T₀-T₁-R^{13a};

V₃ is C1-C4 alkylidene; and

the values for the remainder of the variables and their preferred values are as described for Structural Formula (I).

Preferably, V₃ is C1-C4 alkylidene and T₀ is absent. More preferably, V₃ is C1-C4 alkylidene, T₀ is absent, and T₁ is —O— or —N(R⁶)—.

In a seventh more preferred embodiment, the Chk-1 inhibitor of the present invention is represented by Structural Formula (I), (II), (III) or (IV);

G₂ is C1-C4 alkyl optionally substituted with fluoro or C3-C8 cycloalkyl, wherein the cycloalkyl group is optionally substituted with halo or alkyl;

R¹³is —CN, —OR¹², —NR¹¹R¹², —NHC(O)R¹², —NHC(O)OR¹², —NHC(O)NR¹¹R¹², —NHC(O)OR¹², or —OC(O)R¹²;

R^{13a} is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl, and azabicyclopentyl, azabicyclohexyl, azabicycloheptyl, azabicyclooctyl, azabicyclononyl, azabicyclodecyl, diazabicyclohexyl, diazabicycloheptyl, diazabicyclooctyl, diazabicyclononyl, or diazabicyclodecyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl, and thiadiazolyl;

T₀ is absent;

T₁ is —O— or —N(R⁶)—;

G₁ is -V₃-R¹³, -V₃-R^{13a}, -T₀-T₁-V₃-R¹³, -T₀-T₁-V₃-R^{13a}, or -T₀-T₁-R¹³a;

V₃ is C1-C4 alkylidene; and

the values for the remainder of the variables and their preferred values are as described for Structural Formula (I) above.

In an eighth more preferred embodiment, the Chk-1 inhibitor of the present invention is represented by Structural Formula (I), (II), (III) or (IV);

G₂ is C1-C4 alkyl optionally substituted with fluoro or C3-C8 cycloalkyl, wherein the cycloalkyl group is optionally substituted with halo or alkyl;

R^{13a} is an optionally substituted non-aromatic heterocyclic group selected from N-pyrrolidinyl, N-piperidinyl, N-morpholinyl, N-piperazinyl, N-thiomorpholinyl, N-azetidinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl and 3-oxo-N-8-azabicyclo[3.2.1]octyl or N-8-azabicyclo[3.2.1]octyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl and thiadiazolyl;

T₀ is absent;

T₁ is —O— or —N(R⁶)—;

G₁ is -V₃-R¹³, -V₃-R^{13a}, -T₀-T₁-V₃-R¹³, -T₀-T₁-V₃-R^{13a}, or -T₀-T₁-R^{13a}; V₃ is C1-C4 alkylene;

R¹³ is —OH, —CN, C1-C3 alkoxy, NH₂, C1-C3 alkylamino, C1-C3 dialkylamino, C1-C3 hydroxyalkyl, or C1-C3 haloalkylamino; and

the values for the remainder of the variables and their preferred values are as described for Structural Formula (I) above.

Preferably, R¹³ is NH₂, C1-C3 alkylamino, or C1-C3 dialkylamino; and R^{13a} is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl, 3-oxo-N-8-azabicyclo[3.2.1]octyl or N-8-azabicyclo[3.2.1]octyl.

In a ninth more preferred embodiment, the Chk-1 inhibitor of the present invention is represented by Structural Formula (I), (II), (III) or (IV);

G₂ is C1-C4 alkyl optionally substituted with fluoro or C3-C8 cycloalkyl, wherein the cycloalkyl group is optionally substituted with halo or alkyl;

R¹³ is —CN, —OR¹², —NR¹¹R¹², —NHC(O)R¹², —NHC(O)OR¹², —NHC(O)NR¹¹R¹², —NHC(O)OR¹², or —OC(O)R¹²;

G₁ is -T₀-Cy-V₄-R¹³, T₀-Cy-V₄-R^{13a}, -T₀-Cy-T₁-V₄-R¹³, -T₀-Cy-T₁-V₄-R^{13a}, T₀-Cy-R¹³, or -T₀-Cy-R^{13a};

T₀ is absent;

V₄ is a C1-C4 alkylidene, alkenylidene or alkynylidene group optionally substituted with C1-C3 alkyl; and

the values for the remainder of the variables and their preferred values are as described for Structural Formula (I).

Preferably, V₄ is C1-C4 alkylidene. More preferably V₄ is C1-C4 alkylidene and T₀ is absent. Even more preferably, V₄ is C1-C4 alkylidene, T₀ is absent, and T₁ is —O— or —N(R⁶)—.

In a tenth more preferred embodiment, the Chk-1 inhibitor of the present invention is represented by Structural Formula (I), (II), (III) or (IV);

G₂ is C1-C4 alkyl optionally substituted with fluoro or C3-C8 cycloalkyl, wherein the cycloalkyl group is optionally substituted with halo or alkyl;

R¹³ is —CN, —OR¹², —NR¹¹R¹², —NHC(O)R¹², —NHC(O)OR¹², —NHC(O)NR¹¹R¹², —NHC(O)OR¹², or —OC(O)R¹²;

R^{13a} is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl, and azabicyclopentyl, azabicyclohexyl, azabicycloheptyl, azabicyclooctyl, azabicyclononyl, azabicyclodecyl, diazabicyclohexyl, diazabicycloheptyl, diazabicyclooctyl, diazabicyclononyl, or diazabicyclodecyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl, and thiadiazolyl;

T₀ is absent;

T₁ is —O— or —N(R⁶)—;

G₁ is -T₀-Cy-V₄-R¹³, -T₀-Cy-V₄-R^{13a}, -T₀-Cy-T₁-V₄-R¹³, -T₀-Cy-T₁-V₄-R^{13a}, T₀-Cy-R¹³, or -T₀-Cy-R^{13a};

V₄ is C1-C4 alkylidene;

Cy is an optionally substituted arylene group or an optionally substituted non-aromatic heterocyclene or non-aromatic carbocyclene group; and

the values for the remainder of the variables and their preferred values are as described for Structural Formula (I) above.

Preferably Cy is an optionally substituted phenylene, pyrrolylene, thienylene, furanylene, imidazolylene, triazolylene, tetrazolylene oxazolylene, isoxazolylene, oxadiazolylene, pyrazolylene, pyridinylene, pyrimidylene, pyrazinylene, thiazolylene, cyclopropylene, cyclopentylene, cyclohexylene, cycloheptylene, piperidinylene, piperazinylene, pyrrolidinylene, pyrazolidinylene, imidazolidinylene, tetrahydrofuranylene, tetrahydrothienylene, isooxazolidinylene, oxazolidinylene, isothiazolidinylene, thiazolidinylene, oxathiolanylene, dioxolanylene, or dithiolanylene.

More preferably, Cy is [2,5]thienylene or [2,5]furanylene.

In an eleventh more preferred embodiment, the Chk-1 inhibitor of the present invention is represented by Structural Formula (I), (II), (III) or (IV);

G₂ is C1-C4 alkyl optionally substituted with fluoro or C3-C8 cycloalkyl, wherein the cycloalkyl group is optionally substituted with halo or alkyl;

R^{13a} is an optionally substituted non-aromatic heterocyclic group selected from N-pyrrolidinyl, N-piperidinyl, N-morpholinyl, N-piperazinyl, N-thiomorpholinyl, N-azetidinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl and 3-oxo-N-8-azabicyclo[3.2.1]octyl or N-8-azabicyclo[3.2.1]octyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl and thiadiazolyl;

T₀ is absent;

T₁ is —O— or —N(R⁶)—;

G₁ is -T₀-Cy-V₄-R¹³, -T₀-Cy-V₄-R^{13a}, -T₀-Cy-T₁-V₄-R¹³, -T₀-Cy-T₁-V₄-R^{13a}, T₀-Cy-R¹³, or -T₀-Cy-R^{13a};

V₄ is a C1-C4 alkylidene;

Cy is an optionally substituted phenylene, pyrrolylene, thienylene, furanylene, imidazolylene, triazolylene, tetrazolylene oxazolylene, isoxazolylene, oxadiazolylene, pyrazolylene, pyridinylene, pyrimidylene, pyrazinylene, thiazolylene, cyclopropylene, cyclopentylene, cyclohexylene, cycloheptylene, piperidinylene, piperazinylene, pyrrolidinylene, pyrazolidinylene, imidazolidinylene, tetrahydrofuranylene, tetrahydrothienylene, isooxazolidinylene, oxazolidinylene, isothiazolidinylene, thiazolidinylene, oxathiolanylene, dioxolanylene, or dithiolanylene;

R¹³ is —OH, —CN, C1-C3 alkoxy, or —NR¹¹R¹², where R¹¹ is —H or a C1-C3 alkyl group and R¹² is —H, an optionally substituted alkyl, or an optionally substituted non-aromatic heterocyclic group or NR¹¹R¹² is an optionally substituted aromatic or non-aromatic nitrogen containing heterocyclic group; and

the values for the remainder of the variables and their preferred values are as described for Structural Formula (I) above.

Preferably, Cy is [2,5]thienylene or [2,5]furanylene; R¹³ is NH₂, C1-C3 alkylamino, or C1-C3 dialkylamino; and R^{13a} is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl, 3-oxo-N-8-azabicyclo[3.2.1]octyl or N-8-azabicyclo[3.2.1]octyl.

More preferably, R¹³ is —NH₂, —NHCH₃, —N(CH₃)₂, —NH(CH₂CH₃), or —N(CH₂CH₃)₂; and R^{13a} is N-pyrrolidinyl, 2,5-dimethyl-N-pyrrolidinyl, N-piperidinyl, N′-methyl-N-piperazinyl, N-tetrahydroisoquinolinyl, N-morpholinyl, 3-oxo-N-8-azabicyclo[3.2.1]octyl or N-8-azabicyclo[3.2.1]octyl.

For the first more preferred embodiment through the eleventh more preferred embodiment, the variables are as described above. Preferably, however:

R³ is —H, methyl, ethyl, n-propyl, iso-propyl, C1-C3 haloalkyl, or V₁-R⁷, wherein V₁ is a covalent bond or a C1-C2 alkylidene optionally substituted with one or two methyl groups or with a spiro cyclopropyl group; and R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl. More preferably, R³ is methyl, or ethyl; or R³ is V₁-R⁷, wherein V₁ is a C1-C2 alkylidene and R⁷ is —OH, —OCH₃; or V₁ is a covalent bond and R⁷ is cyclopropyl, cyclopentyl, furyl or tetrahydrofuryl; and/or

each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl). More preferably, each R⁵ are independently —H, halogen, C1-C3 alkyl, C1-C3 haloalkyl, C1-C3 alkoxy, or C1-C3 haloalkoxy.

Even more preferably R³ is methyl, or ethyl; or R³ is V₁-R⁷, wherein V₁ is a C1-C2 alkylidene and R⁷ is —OH, —OCH₃; or V₁ is a covalent bond and R⁷ is cyclopropyl, cyclopentyl, furyl or tetrahydrofuryl, and/or R⁴ and each R⁵ are independently —H, halogen, C1-C3 alkyl, C1-C3 haloalkyl, C1-C3 alkoxy, or C1-C3 haloalkoxy.

In another preferred embodiment, the Chk-1 inhibitor of the present invention is represented by Structural Formula (VI): 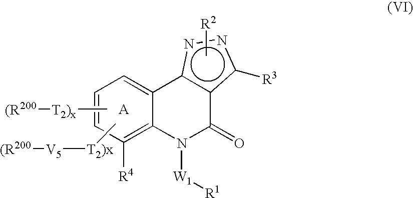
Values and preferred values for the variables in Structural Formula (VI) are as described above for Structural Formula (I).

In a preferred embodiment, the Chk-1 inhibitor of the present invention is represented by Structural Formula (VI):

Ring A is optionally substituted at any one or more substitutable ring carbon atoms;

R¹ is —H, —CONR¹¹R¹², —COOR¹², fluoro, or a cycloalkyl wherein the cycloalkyl is optionally substituted with halo or alkyl and W₁ is a linear C1-C6 alkylidene chain; or R¹ is —OR¹² and W₁ is a linear C2-C6 alkylidene group, wherein the alkylidene group represented by W₁ is optionally substituted with one or more —CH₃ or fluoro groups; or -W₁-R¹ is —H; and

the values for all other variables and their preferred values are as described above for Structural Formula (I).

In another preferred embodiment, the Chk-1 inhibitor of the present invention is represented by Structural Formula (VII): 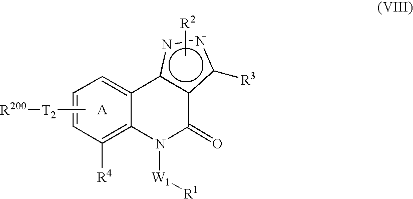
or a pharmaceutically acceptable salt thereof. Values and preferred values for the variables in Structural Formula (VII) are as described above for Structural Formula (I).

In a preferred embodiment, the Chk-1 inhibitor of the present invention is represented by Structural Formula (VII):

R¹ is —H, —CONR¹¹R¹², —COOR¹², fluoro, or a cycloalkyl wherein the cycloalkyl is optionally substituted with halo or alkyl and W₁ is a linear C1-C6 alkylidene chain; or R¹ is —OR¹² and W₁ is a linear C2-C6 alkylidene group, wherein the alkylidene group represented by W₁ is optionally substituted with one or more —CH₃ or fluoro groups; or -W₁-R¹ is —H;

R⁴ is —H, C1-C3 alkyl, C1-C3 haloalkyl, halogen, hydroxy, C1-C3 alkoxy, C1-C3 haloalkoxy, —NH₂, C1-C3 alkylamino, C1-C3 dialkylamino, —NHC(O)H, —NHC(O)(C1-C3 alkyl), —C(O)NH₂, —C(O)NH(C1-C3 alkyl) or —C(O)N(C1-C3 alkyl)₂; and

the values for all other variables and their preferred values are as described above for Structural Formula (I).

In another preferred embodiment the Chk-1 inhibitor of the present invention is represented by Structural Formula (VIII):

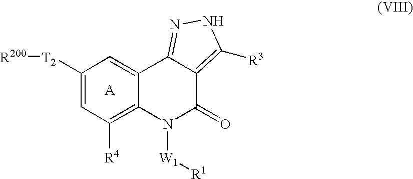

The values and preferred values for the variables in Structural Formula (VIII) are as described above for Structural Formula (I).

In a preferred embodiment, the Chk-1 inhibitor of the present invention is represented by Structural Formula (VII) or (VIII);

R¹ is —H, —CONR¹¹R¹², —COOR¹², fluoro, or a cycloalkyl wherein the cycloalkyl is optionally substituted with halo or alkyl and W₁ is a linear C1-C6 alkylidene chain; or R¹ is —OR¹² and W₁ is a linear C2-C6 alkylidene group, wherein the alkylidene group represented by W₁ is optionally substituted with one or more —CH₃ or fluoro groups; or -W₁-R¹ is —H. Preferably, W₁ is a linear C1-C4 alkylidene chain optionally substituted with one or more —CH₃ or fluoro groups and R¹ is —H, fluoro or a cycloalkyl wherein the cycloalkyl is optionally substituted with halo or alkyl;

T₂ is a covalent bond, —S(O), —SO₂—, —C(O)—, —OC(O)—, —N(R⁶)C(O)—, or —N(R⁶)SO₂. Preferably, T₂ is a covalent bond; and

the values for all other variables and their preferred values are as described above for Structural Formula (I).

In another preferred embodiment the Chk-1 inhibitor of the present invention is represented a Structural Formula selected from (IX) and (X):

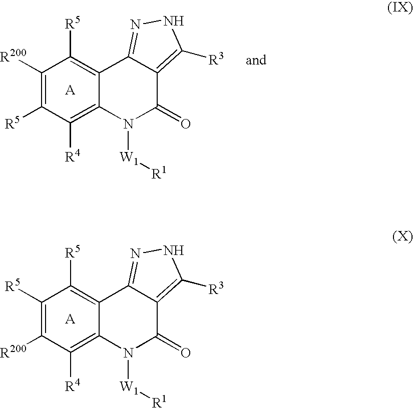

Each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl). The values and preferred values for the remaining variables in Structural Formulas (IX) and (X) are as described above for Structural Formula (I).

In a twelfth more preferred embodiment, the Chk-1 inhibitor of the present invention is represented by Structural Formula (VI), (VII), (VIII), (IX) or (X);

R¹ is —H, —CONR¹¹R¹², —COOR¹², fluoro, or a cycloalkyl wherein the cycloalkyl is optionally substituted with halo or alkyl and W₁ is a linear C1-C6 alkylidene chain; or R¹ is —OR¹² and W₁ is a linear C2-C6 alkylidene group, wherein the alkylidene group represented by W₁ is optionally substituted with one or more —CH₃ or fluoro groups; or -W₁-R¹ is —H. Preferably, W₁ is a linear C1-C4 alkylidene chain optionally substituted with one or more —CH₃ or fluoro groups and R¹ is —H, fluoro or a cycloalkyl wherein the cycloalkyl is optionally substituted with halo or alkyl;

each R⁵, when present, is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl); and

the values for all other variables and their preferred values are as described above for Structural Formula (I).

In a thirteenth more preferred embodiment, the Chk-1 inhibitor of the present invention is represented by Structural Formula (VI), (VII), (VIII), (IX) or (X);

R²⁰⁰ is —C≡CR²⁰¹, —CH═CHR²⁰¹, —C≡C(C(R²⁰R²⁰))ₚR²⁰², or —CH═CH—(C(R²⁰R²⁰))ₚR²⁰²;

R²⁰¹ is —H, alkyl, haloalkyl, hydroxyalkyl, CO₂R⁵¹, or an optionally substituted aromatic group or non-aromatic heterocyclic group;

R²⁰² is —H, —CN, —OR⁵¹, —OC(O)NR⁵¹R⁵², —OC(O)R⁵¹, —NR⁵¹R⁵², —C(O)NR⁵¹R⁵², —NR⁵¹C(O)R⁵¹, —NR⁵¹C(O)NR⁵¹R⁵², —NR⁵¹C(O)OR⁵¹, —NR⁵¹S(O)₂R^{x}, —S(O)₂NR⁵¹, —CO₂R⁵¹ or an optionally substituted aromatic group or non-aromatic heterocyclic group;

each R²⁰ is independently —H or C1-C3 alkyl;

R^{x} is alkyl or an optionally substituted aromatic group or non-aromatic heterocyclic group;

p is 1 or 2; and

the values for all other variables and their preferred values are as described above for the twelfth more preferred embodiment.

In a fourteenth more preferred embodiment, the Chk-1 inhibitor of the present invention is represented by Structural Formula ((VI), (VII), (VIII), (IX) or (X);

R³ is —H, methyl, ethyl, n-propyl, iso-propyl, C1-C3 haloalkyl, or V₁-R⁷, wherein V₁ is a covalent bond or a C1-C2 alkylidene optionally substituted with one or two methyl groups or with a spiro cyclopropyl group; R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl; and

the values for all other variables and their preferred values are as described above for the thirteenth more preferred embodiment.

In a fifteenth more preferred embodiment, the Chk-1 inhibitor of the present invention is represented by Structural Formula (VI), (VII), (VIII), (IX) or (X);

R²⁰¹ is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-azetidinyl 3-pyrrolidinyl, 3-piperidinyl, 3-morpholinyl, 3-thiomorpholinyl, 3-azetidinyl, 4-piperidinyl, tetrahydroquinolinyl, tetrahydroisoquinolinyl, N-tetrahydroquinolinyl, N-tetrahydroisoquinolinyl 3-oxo-N-8-azabicyclo[3.2.1]octyl or N-8-azabicyclo[3.2.1]octyl;

R²⁰² is —CN, —OH, C1-C3 alkoxy, —NH₂, C1-C3 alkylamino, C1-C3 dialkylamino, an optionally substituted non-aromatic heterocyclic group selected from N-pyrrolidinyl, N-piperidinyl, N-morpholinyl, N-piperazinyl, N-thiomorpholinyl, 2-pyrrolidinyl, 2-piperidinyl, 2-piperazinyl, 2-morpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl, 3-oxo-N-8-azabicyclo[3.2.1]octyl or N-8-azabicyclo[3.2.1]octyl. Preferably, R²⁰² is NH₂, C1-C3 alkylamino, C1-C3 dialkylamino, an optionally substituted non-aromatic heterocyclic group selected from N-pyrrolidinyl, N-piperidinyl, N-piperazinyl, N-morpholinyl, N-azetidinyl, N-thiomorpholinyl, 2-pyrrolidinyl, 2-piperidinyl, 2-piperazinyl, 2-morpholinyl, 2-azetidinyl, 3-pyrrolidinyl, 3-piperidinyl, 3-morpholinyl, 3-thiomorpholinyl, 3-azetidinyl, 4-piperidinyl, tetrahydroquinolinyl, tetrahydroisoquinolinyl, N-tetrahydroquinolinyl and N-tetrahydroisoquinolinyl, 3-oxo-N-8-azabicyclo[3.2.1]octyl or N-8-azabicyclo[3.2.1]octyl; and

the values for all other variables and their preferred values are as described above for the fourteenth more preferred embodiment.

In a sixteenth more preferred embodiment, the Chk-1 inhibitor of the present invention is represented by Structural Formulas (VI), (VII), (VIII), (IX) or (X);

R²⁰⁰ is —C≡C—R²⁰³ or —C═CHR²⁰³;

R²⁰³ has the formula -V₆-R⁶⁰, -V₆-R⁶¹, -T₁₁-V₆-R⁶⁰, or -T₁₁-V₆-R⁶¹;

V₆ is a C1-C4 alkylidene, wherein V₆ is optionally substituted with alkyl, halo, haloalkyl, alkoxy, hydroxy, NR¹¹R¹² or oxo;

T₁₁ is —S(O)—, —S(O)₂—, —C(O)—, —C(O)O—, —C(O)N(R⁶)—, or —SO₂N(R⁶)—;

R⁶⁰ is —OR¹², —CN, —COOR¹², —NR¹¹R¹², —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —O—C(O)—OR¹², —OC(O)—NR¹¹R¹², —NR¹¹CO—CH(OR⁶²)—R¹², —NR¹¹CO—CH(NR⁶²R⁶²)—R¹², —NR¹¹CO—(CH₂)_{z}CH(NR⁶²R⁶²)—R¹², —OC(O)—CH(OR⁶²)—R¹², —OC(O)—CH(NR⁶²R⁶²)—R¹², —NR¹¹CO—C(R⁶³R⁶³)—OR¹², —NR¹¹CO—C(R⁶³R⁶³)—NR¹¹R¹², —OC(O)—C(R⁶³R⁶³)—OR¹², —OC(O)—C(R⁶³R⁶³)—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², —C(O)NR¹¹R¹², —NHC(O)NR¹¹R¹², or —C(═NR¹¹)—NR¹¹R¹²;

R⁶¹ is an optionally substituted nitrogen-containing heteroaromatic group or a nitrogen-containing non-aromatic heterocyclic group; and

the values for all other variables and their preferred values are as described above for the twelfth more preferred embodiment.

In a seventeenth more preferred embodiment, the Chk-1 inhibitor of the present invention is represented by Structural Formula (IX) or (X);

R⁶⁰ is —CN, —OR¹², —NR¹¹R¹², —NHC(O)R¹², —NHC(O)OR¹², —NHC(O)NR¹¹R¹², —NHC(O)OR¹², or —OC(O)R¹²;

R⁶¹ is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl and azabicyclopentanyl, azabicyclohexanyl, azabicycloheptanyl, azabicyclononanyl, azabicyclodecanyl, diazabicyclohexanyl, diazabicycloheptanyl, diazabicyclooctanyl, diazabicyclononanyl, or diazabicyclodecanyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl and thiadiazolyl; and

the values for all other variables and their preferred values are as described above for the sixteenth more preferred embodiment.

In an eighteenth more preferred embodiment, the Chk-1 inhibitor of the present invention is represented by Structural Formula (IX) or (X);

R⁶⁰ is —OH, —CN, C1-C3 alkoxy, or NR¹¹R¹², where R¹¹ is —H or a C1-C3 alkyl group and R¹² is —H, an optionally substituted alkyl, or an optionally substituted non-aromatic heterocyclic group, or NR¹¹R¹² is an optionally substituted aromatic or non-aromatic nitrogen containing heterocyclic group. Preferably, R⁶⁰ is NH₂, C1-C3 alkylamino, or C1-C3 dialkylamino. More preferably, R⁶⁰ is —NH₂, —NHCH₃, —N(CH₃)₂, —NH(CH₂CH₃), or —N(CH₂CH₃)₂;

R⁶¹ is an optionally substituted non-aromatic heterocyclic group selected from N-pyrrolidinyl, N-piperidinyl, N-morpholinyl, N-piperazinyl, N-thiomorpholinyl, N-azetidinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl and 3-oxo-N-8-azabicyclo[3.2.1]octyl or N-8-azabicyclo[3.2.1]octyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl and thiadiazolyl. Preferably, R⁶¹ is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl N-tetrahydroisoquinolinyl 3-oxo-N-8-azabicyclo[3.2.1]octyl or N-8-azabicyclo[3.2.1]octyl. More preferably, R⁶¹ is N-pyrrolidinyl, 2,5-dimethyl-N-pyrrolidinyl, N-piperidinyl, N′-methyl-N-piperazinyl, N-tetrahydroisoquinolinyl, N-morpholinyl, 3-oxo-N-8-azabicyclo[3.2.1]octyl or N-8-azabicyclo[3.2.1]octyl; and

the values for all other variables and their preferred values are as described above for the seventeenth more preferred embodiment.

In an nineteenth more preferred embodiment, the Chk-1 inhibitor of the present invention is represented by Structural Formula (IX) or (X)

R³ is methyl, or ethyl; or R³ is V₁-R⁷, wherein V₁ is a C1-C2 alkylidene and R⁷ is —OH, —OCH₃; or wherein V₁ is a covalent bond and R⁷ is -cyclopropyl, cyclopentyl, furyl or tetrahydrofuryl;

R⁴ and each R⁵ is independently —H, halogen, —CH₃, halomethyl, —OCH₃, or haloalkoxy; and

the values for all other variables and their preferred values are as described above for the thirteenth, fifteenth, sixteenth, seventeenth or eighteenth more preferred embodiments.

In a twentieth more preferred embodiment, the Chk-1 inhibitor of the present invention is represented by a Structural Formula selected from (XI) and (XII):

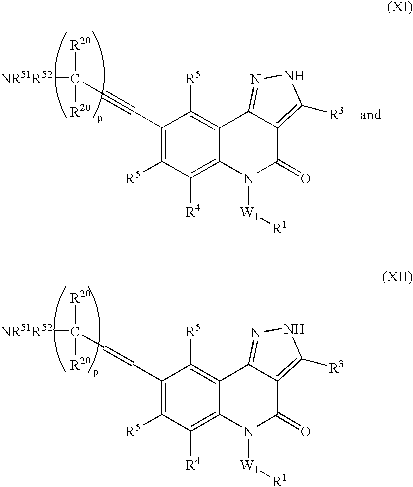

Each R²⁰ is independently —H or C1-C3 alkyl.

p is 1 or 2.

All other variables and preferred variables are as described above for Structural Formula (I).

In a twenty first more preferred embodiment, the Chk-1 inhibitor of the present invention is represented by Structural Formula (XI) or (XII);

R¹ is —H, —CONR¹¹R¹², —COOR¹², fluoro, or a cycloalkyl optionally substituted with halo or alkyl and W₁ is a linear C1-C6 alkylidene chain; R¹ is —OR¹² and W₁ is a linear C2-C6 alkylidene group, wherein the alkylidene group represented by W₁ is optionally substituted with one or more —CH₃ or fluoro groups; or -W₁-R¹ is —H;

R³ is —H, methyl, ethyl, n-propyl, iso-propyl, C1-C3 haloalkyl, or V₁-R⁷, wherein V₁ is a covalent bond or a C1-C2 alkylidene optionally substituted with one or two methyl groups or with a spiro cyclopropyl group; R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl;

each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl); and

the values for all other variables and their preferred values are as described above for Structural Formula (I).

In a twenty second more preferred embodiment, the Chk-1 inhibitor of the present invention is represented by Structural Formula (XI) or (XII);

W₁ is a linear C1-C4 alkylidene chain optionally substituted with one or more —CH₃ or fluoro groups and R¹ is —H, fluoro or a cycloalkyl optionally substituted with halo or alkyl;

R³ is methyl, or ethyl; or R³ is V₁-R⁷, wherein V₁ is a C1-C2 alkylidene and R⁷ is —OH, —OCH₃; or wherein V₁ is a covalent bond and R⁷ is -cyclopropyl, cyclopentyl, furyl or tetrahydrofuryl;

R⁴ and each R⁵ is independently —H, halogen, —CH₃, halomethyl, —OCH₃, or haloalkoxy;

the values for all other variables and their preferred values are as described above for Structural Formula (I).

Specific examples of Chk-1 inhibitors of the present invention are provided below in Table 1.

TABLE 1

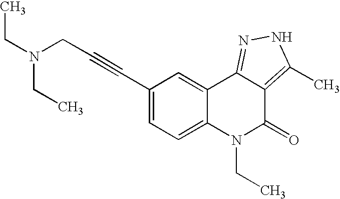

I-1

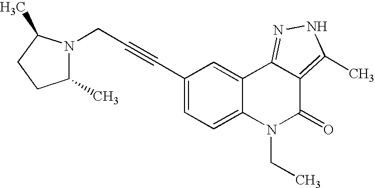

I-2

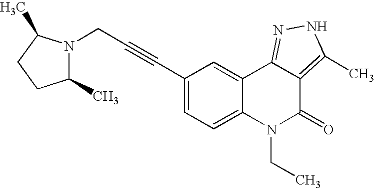

I-3

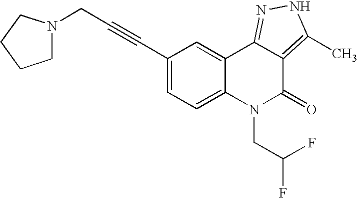

I-4

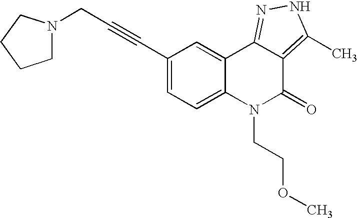

I-5

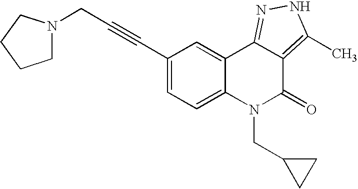

I-6

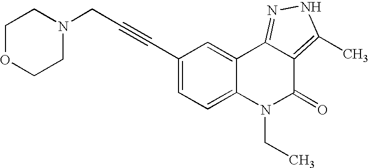

I-7

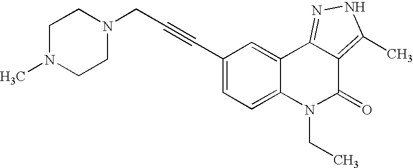

I-8

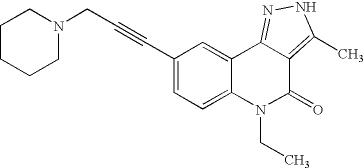

I-9

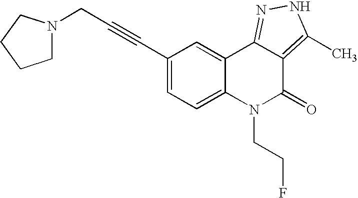

I-10

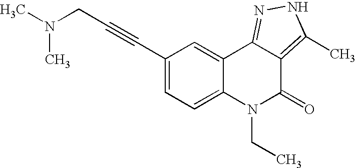

I-11

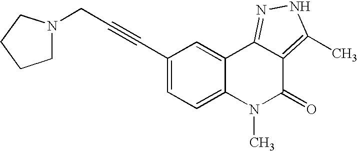

I-12

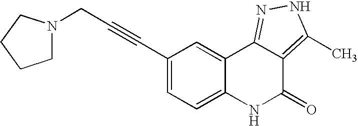

I-13

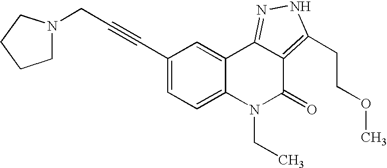

I-14

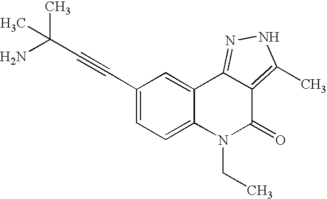

I-15

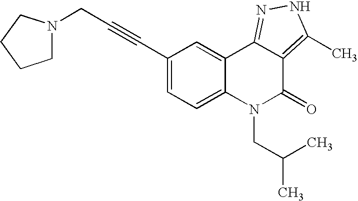

I-16

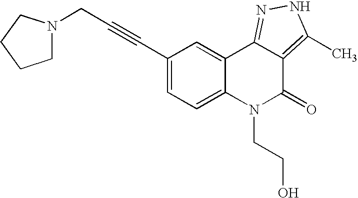

I-17

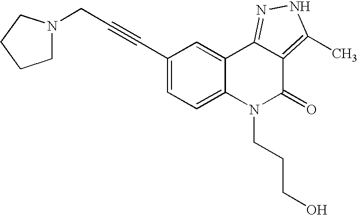

I-18

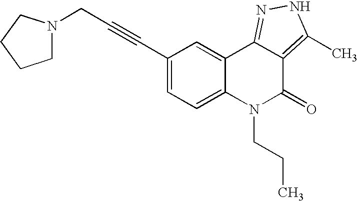

I-19

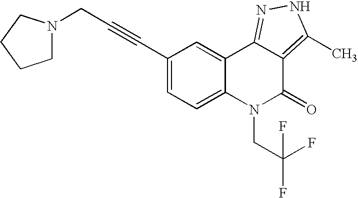

I-20

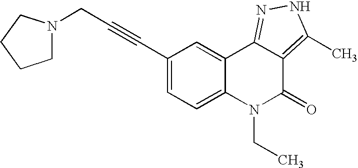

I-21

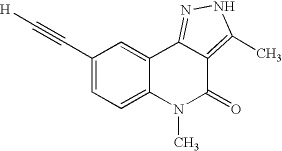

I-22

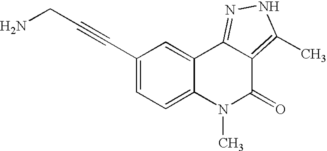

I-23

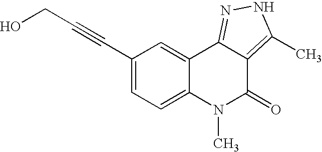

I-24

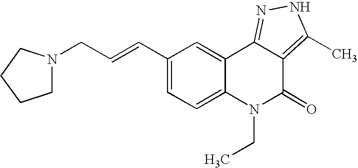

I-25

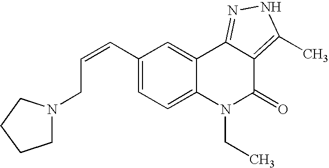

I-26

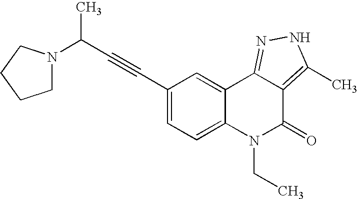

I-27

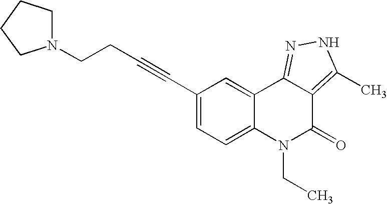

I-28

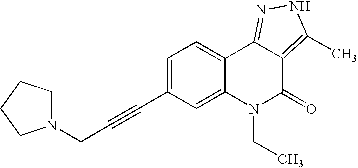

I-29

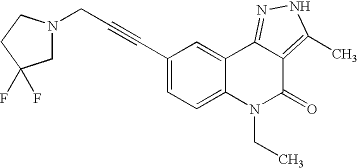

I-30

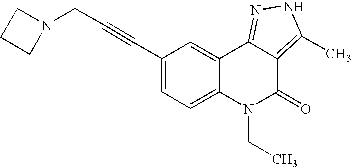

I-31

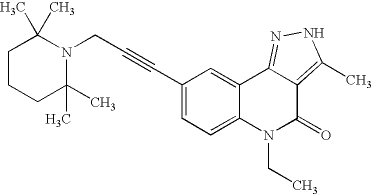

I-32

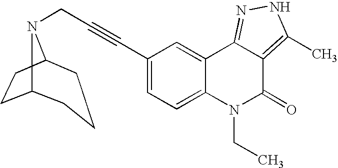

I-33

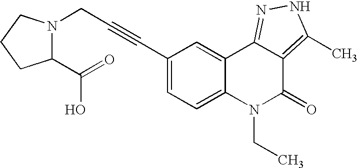

I-34

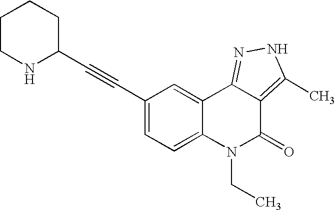

I-35

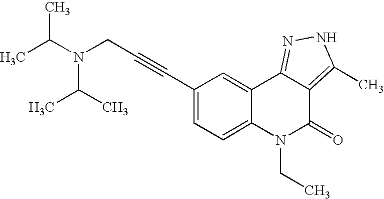

I-36

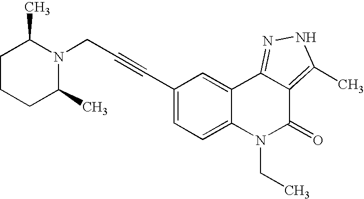

I-37

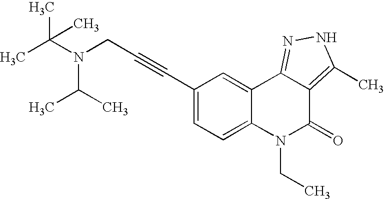

I-38

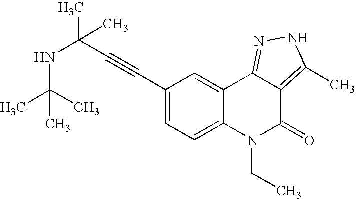

I-39

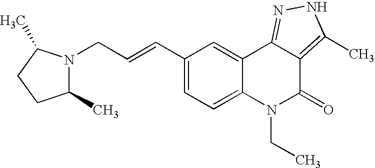

I-40

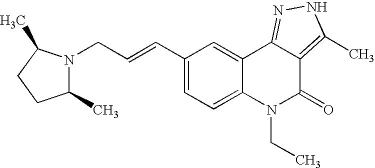

I-41

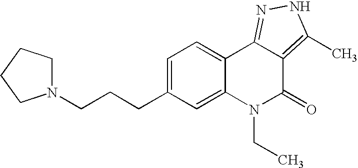

I-42

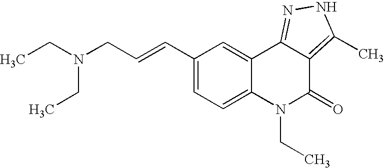

I-43

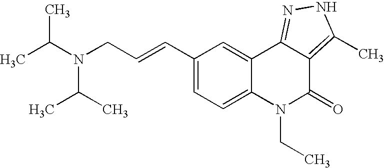

I-44

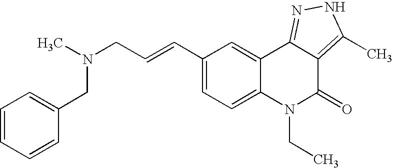

I-45

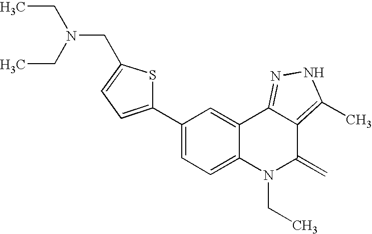

I-46

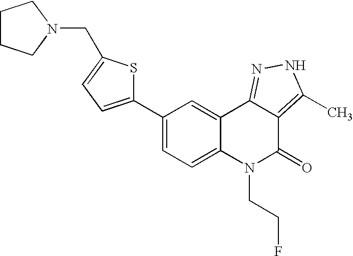

I-47

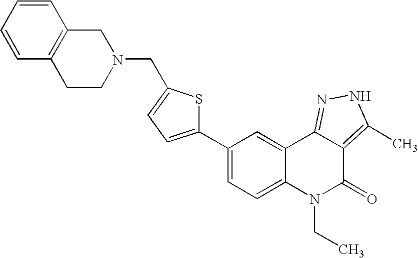

I-48

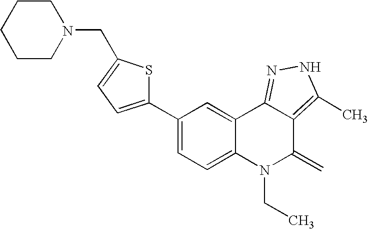

I-49

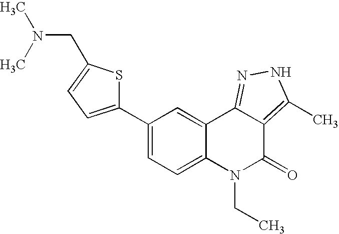

I-50

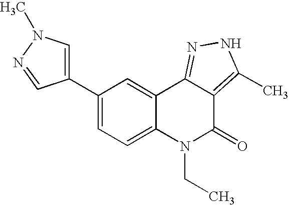

I-51

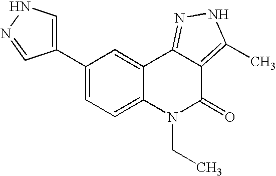

I-52

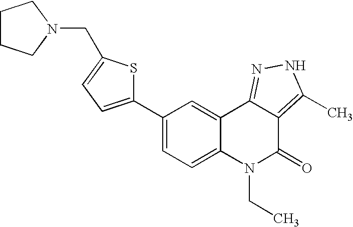

I-53

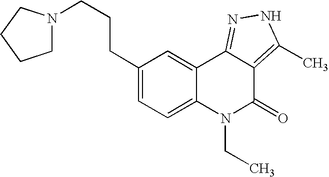

I-54

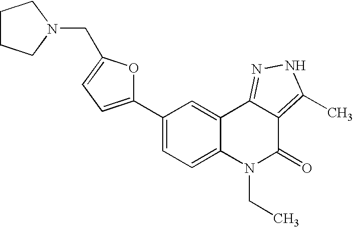

I-55

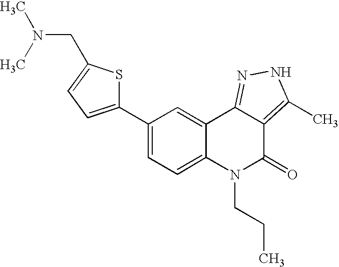

I-56

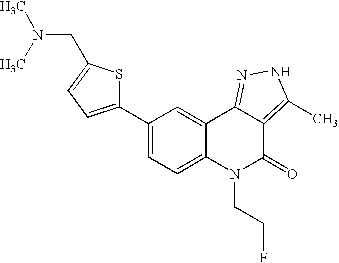

I-57

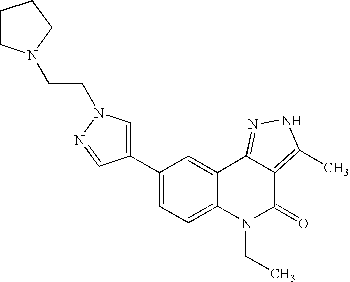

I-58

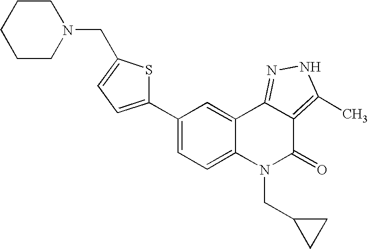

I-59

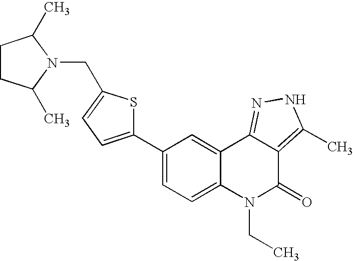

I-60

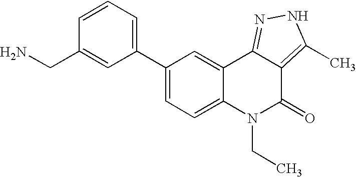

I-61

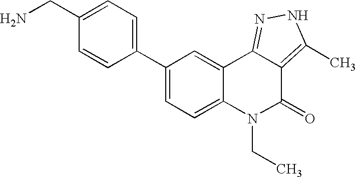

I-62

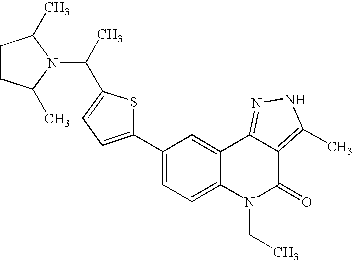

I-63

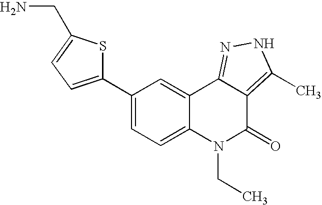

I-64

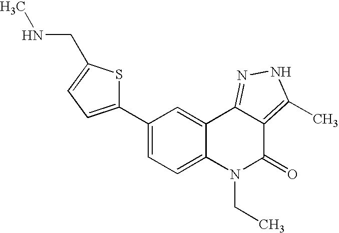

I-65

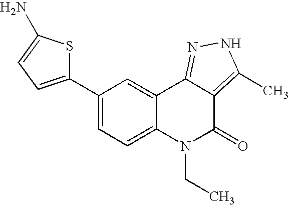

I-66

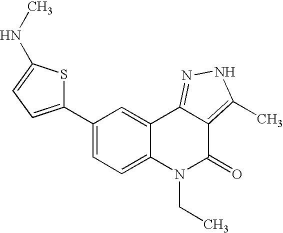

I-67

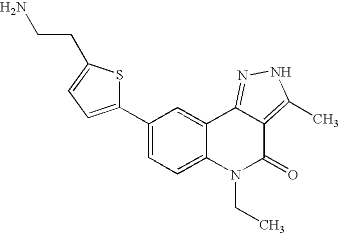

I-68

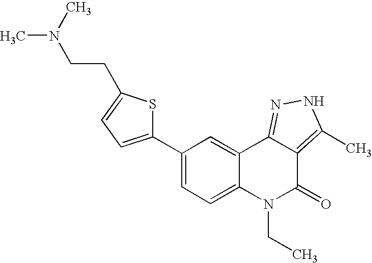

I-69

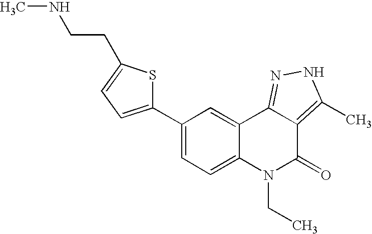

I-70

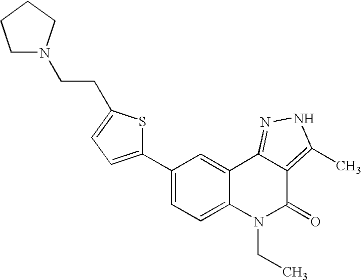

I-71

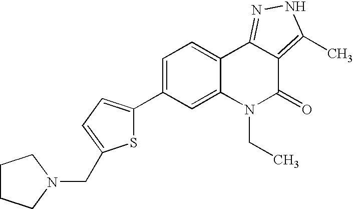

I-72

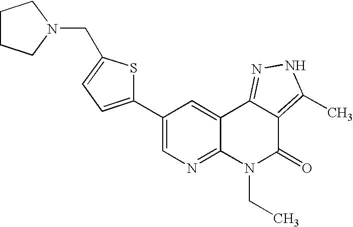

I-73

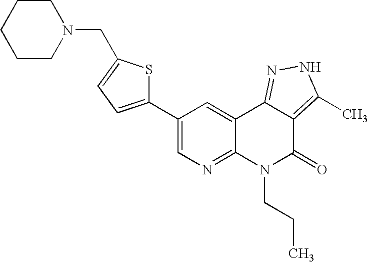

I-74

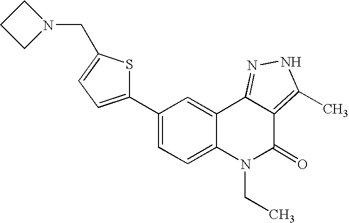

I-75

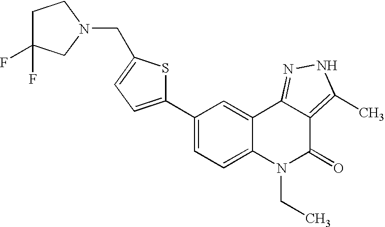

I-76

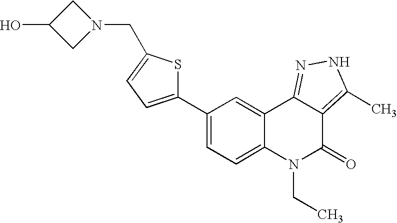

I-77

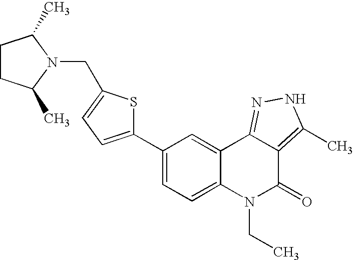

I-78

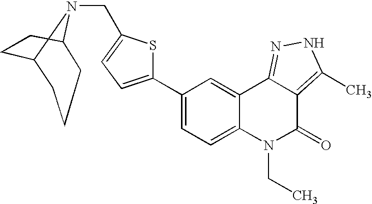

I-79

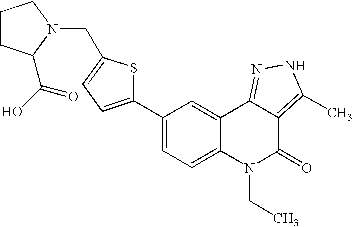

I-80

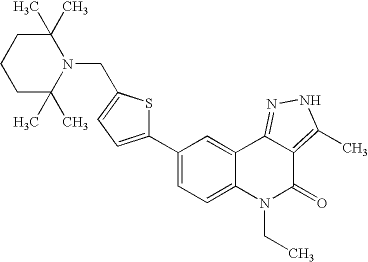

I-81

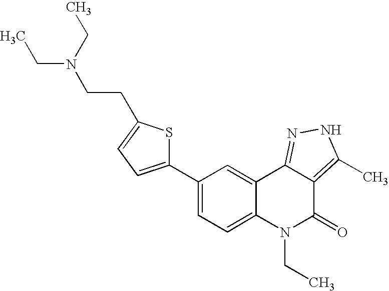

I-82

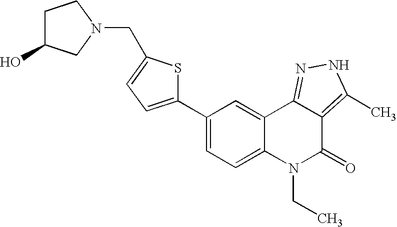

I-83

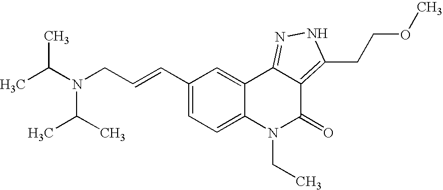

I-84

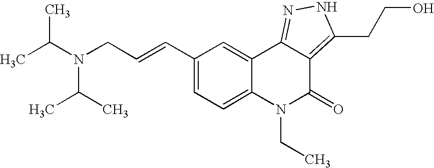

I-85

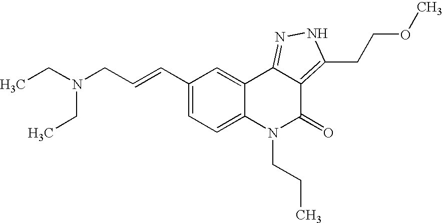

I-86

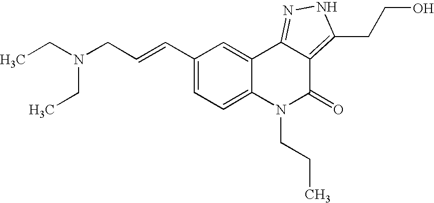

I-87

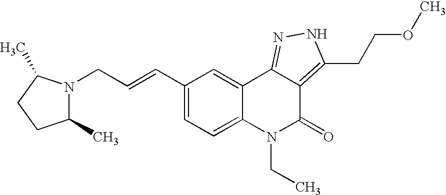

I-88

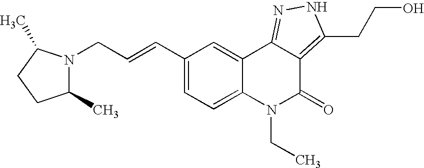

I-89

The Chk-1 inhibitors depicted in Table 1 above also may be identified by the following chemical names:

| | Chemical Name |
|---|---|
| I-1: | 8-[3-(diethylamino)prop-1-yn-1-yl]-5-ethyl-3-methyl-2,5-dihydro-4H- |
| | pyrazolo[4,3-c]quinolin-4-one |
| I-2: | 8-{3-[(2S,5S)-2,5-dimethylpyrrolidin-1-yl]prop-1-yn-1-yl}-5-ethyl-3-methyl-2,5-dihydro- |
| | 4H-pyrazolo[4,3-c]quinolin-4-one |
| I-3: | 8-{3-[(2R,5S)-2,5-dimethylpyrrolidin-1-yl]prop-1-yn-1-yl}-5-ethyl-3-methyl- |
| | 2,5-dihydro-4H-pyrazolo[4,3-c]quinolin-4-one |
| I-4: | 5-(2,2-difluoroethyl)-3-methyl-8-(3-pyrrolidin-1-ylprop-1-yn-1-yl)-2,5- |
| | dihydro-4H-pyrazolo[4,3-c]quinolin-4-one |
| I-5: | 5-(2-methoxyethyl)-3-methyl-8-(3-pyrrolidin-1-ylprop-1-yn-1-yl)-2,5- |
| | dihydro-4H-pyrazolo[4,3-c]quinolin-4-one |
| I-6: | 5-(cyclopropylmethyl)-3-methyl-8-(3-pyrrolidin-1-ylprop-1-yn-1-yl)-2,5- |
| | dihydro-4H-pyrazolo[4,3-c]quinolin-4-one |
| I-7: | 5-ethyl-3-methyl-8-(3-morpholin-4-ylprop-1-yn-1-yl)-2,5-dihydro-4H- |
| | pyrazolo[4,3-c]quinolin-4-one |
| I-8: | 5-ethyl-3-methyl-8-[3-(4-methylpiperazin-1-yl)prop-1-yn-1-yl]-2,5-dihydro- |
| | 4H-pyrazolo[4,3-c]quinolin-4-one |
| I-9: | 5-ethyl-3-methyl-8-(3-piperidin-1-ylprop-1-yn-1-yl)-2,5-dihydro-4H- |
| | pyrazolo[4,3-c]quinolin-4-one |
| I-10: | 5-(2-fluoroethyl)-3-methyl-8-(3-pyrrolidin-1-ylprop-1-yn-1-yl)-2,5-dihydro- |
| | 4H-pyrazolo[4,3-c]quinolin-4-one |
| I-11: | 8-[3-(dimethylamino)prop-1-yn-1-yl]-5-ethyl-3-methyl-2,5-dihydro-4H- |
| | pyrazolo[4,3-c]quinolin-4-one |
| I-12: | 3,5-dimethyl-8-(3-pyrrolidin-1-ylprop-1-yn-1-yl)-2,5-dihydro-4H- |
| | pyrazolo[4,3-c]quinolin-4-one |
| I-13: | 3-methyl-8-(3-pyrrolidin-1-ylprop-1-yn-1-yl)-2,5-dihydro-4H-pyrazolo[4,3- |
| | c]quinolin-4-one |
| I-14: | 5-ethyl-3-(2-methoxyethyl)-8-(3-pyrrolidin-1-ylprop-1-yn-1-yl)-2,5-dihydro- |
| | 4H-pyrazolo[4,3-c]quinolin-4-one |
| I-15: | 8-(3-amino-3-methylbut-1-yn-1-yl)-5-ethyl-3-methyl-2,5-dihydro-4H- |
| | pyrazolo[4,3-c]quinolin-4-one |
| I-16: | 5-isobutyl-3-methyl-8-(3-pyrrolidin-1-ylprop-1-yn-1-yl)-2,5-dihydro-4H- |
| | pyrazolo[4,3-c]quinolin-4-one |
| I-17: | 5-(2-hydroxyethyl)-3-methyl-8-(3-pyrrolidin-1-ylprop-1-yn-1-yl)-2,5- |
| | dihydro-4H-pyrazolo[4,3-c]quinolin-4-one |
| I-18: | 5-(3-hydroxypropyl)-3-methyl-8-(3-pyrrolidin-1-ylprop-1-yn-1-yl)-2,5- |
| | dihydro-4H-pyrazolo[4,3-c]quinolin-4-one |
| I-19: | 3-methyl-5-propyl-8-(3-pyrrolidin-1-ylprop-1-yn-1-yl)-2,5-dihydro-4H- |
| | pyrazolo[4,3-c]quinolin-4-one |
| I-20: | 3-methyl-8-(3-pyrrolidin-1-ylprop-1-yn-1-yl)-5-(2,2,2-trifluoroethyl)-2,5- |
| | dihydro-4H-pyrazolo[4,3-c]quinolin-4-one |
| I-21: | 5-ethyl-3-methyl-8-(3-pyrrolidin-1-ylprop-1-yn-1-yl)-2,5-dihydro-4H- |
| | pyrazolo[4,3-c]quinolin-4-one |
| I-22: | 8-ethynyl-3,5-dimethyl-2,5-dihydro-4H-pyrazolo[4,3-c]quinolin-4-one |
| I-23: | 8-(3-aminoprop-1-yn-1-yl)-3,5-dimethyl-2,5-dihydro-4H-pyrazolo[4,3- |
| | c]quinolin-4-one |
| I-24: | 8-(3-hydroxyprop-1-yn-1-yl)-3,5-dimethyl-2,5-dihydro-4H-pyrazolo[4,3- |
| | c]quinolin-4-one |
| I-25: | 5-ethyl-3-methyl-8-[(1E)-3-pyrrolidin-1-ylprop-1-en-1-yl]-2,5-dihydro-4H- |
| | pyrazolo[4,3-c]quinolin-4-one |
| I-26: | 5-ethyl-3-methyl-8-[(1Z)-3-pyrrolidin-1-ylprop-1-en-1-yl]-2,5-dihydro-4H- |
| | pyrazolo[4,3-c]quinolin-4-one |
| I-27: | 5-ethyl-3-methyl-8-(3-pyrrolidin-1-ylbut-1-yn-1-yl)-2,5-dihydro-4H- |
| | pyrazolo[4,3-c]quinolin-4-one |
| I-28: | 5-ethyl-3-methyl-8-(4-pyrrolidin-1-ylbut-1-yn-1-yl)-2,5-dihydro-4H- |
| | pyrazolo[4,3-c]quinolin-4-one |
| I-29: | 5-ethyl-3-methyl-7-(3-pyrrolidin-1-ylprop-1-yn-1-yl)-2,5-dihydro-4H- |
| | pyrazolo[4,3-c]quinolin-4-one |
| I-30: | 8-[3-(3,3-difluoropyrrolidin-1-yl)prop-1-yn-1-yl]-5-ethyl-3-methyl-2,5- |
| | dihydro-4H-pyrazolo[4,3-c]quinolin-4-one |
| I-31: | 8-(3-azetidin-1-ylprop-1-yn-1-yl)-5-ethyl-3-methyl-2,5-dihydro-4H- |
| | pyrazolo[4,3-c]quinolin-4-one |
| I-32: | 5-ethyl-3-methyl-8-[3-(2,2,6,6-tetramethylpiperidin-1-yl)prop-1-yn-1-yl]-2,5- |
| | dihydro-4H-pyrazolo[4,3-c]quinolin-4-one |
| I-33: | 8-[3-(8-azabicyclo[3.2.1]oct-8-yl)prop-1-yn-1-yl]-5-ethyl-3-methyl-2,5- |
| | dihydro-4H-pyrazolo[4,3-c]quinolin-4-one |
| I-34: | 1-[3-(5-ethyl-3-methyl-4-oxo-4,5-dihydro-2H-pyrazolo[4,3-c]quinolin-8- |
| | yl)prop-2-yn-1-yl]pyrrolidine-2-carboxylic acid |
| I-35: | 5-ethyl-3-methyl-8-(piperidin-2-ylethynyl)-2,5-dihydro-4H-pyrazolo[4,3- |
| | c]quinolin-4-one |
| I-36: | 8-[3-(diisopropylamino)prop-1-yn-1-yl]-5-ethyl-3-methyl-2,5-dihydro-4H- |
| | pyrazolo[4,3-c]quinolin-4-one |
| I-37: | 8-{3-[(2R,6S)-2,6-dimethylpiperidin-1-yl]prop-1-yn-1-yl}-5-ethyl-3-methyl- |
| | 2,5-dihydro-4H-pyrazolo[4,3-c]quinolin-4-one |
| I-38: | 8-{3-[tert-butyl(isopropyl)amino]prop-1-yn-1-yl}-5-ethyl-3-methyl-2,5- |
| | dihydro-4H-pyrazolo[4,3-c]quinolin-4-one |
| I-39: | 8-[3-(tert-butylamino)-3-methylbut-1-yn-1-yl]-5-ethyl-3-methyl-2,5-dihydro- |
| | 4H-pyrazolo[4,3-c]quinolin-4-one |
| I-40: | 8-{(1E)-3-[(2S,5S)-2,5-dimethylpyrrolidin-1-yl]prop-1-en-1-yl}-5-ethyl-3- |
| | methyl-2,5-dihydro-4H-pyrazolo[4,3-c]quinolin-4-one |
| I-41: | 8-{(1E)-3-[(2R,5S)-2,5-dimethylpyrrolidin-1-yl]prop-1-en-1-yl}-5-ethyl-3- |
| | methyl-2,5-dihydro-4H-pyrazolo[4,3-c]quinolin-4-one |
| I-42: | 5-ethyl-3-methyl-7-(3-pyrrolidin-1-ylpropyl)-2,5-dihydro-4H-pyrazolo[4,3- |
| | c]quinolin-4-one |
| I-43: | 8-[(1E)-3-(diethylamino)prop-1-en-1-yl]-5-ethyl-3-methyl-2,5-dihydro-4H- |
| | pyrazolo[4,3-c]quinolin-4-one |
| I-44: | 8-[(1E)-3-(diisopropylamino)prop-1-en-1-yl]-5-ethyl-3-methyl-2,5-dihydro- |
| | 4H-pyrazolo[4,3-c]quinolin-4-one |
| I-45: | 8-{(1E)-3-[benzyl(methyl)amino]prop-1-en-1-yl}-5-ethyl-3-methyl-2,5- |
| | dihydro-4H-pyrazolo[4,3-c]quinolin-4-one |
| I-46: | 8-{5-[(diethylamino)methyl]-2-thienyl}-5-ethyl-3-methyl-2,5-dihydro-4H- |
| | pyrazolo[4,3-c]quinolin-4-one |
| I-47: | 5-(2-fluoroethyl)-3-methyl-8-[5-(pyrrolidin-1-ylmethyl)-2-thienyl]-2,5- |
| | dihydro-4H-pyrazolo[4,3-c]quinolin-4-one |
| I-48: | 8-[5-(3,4-dihydroisoquinolin-2(1H)-ylmethyl)-2-thienyl]-5-ethyl-3-methyl- |
| | 2,5-dihydro-4H-pyrazolo[4,3-c]quinolin-4-one |
| I-49: | 5-ethyl-3-methyl-4-methylene-8-[5-(piperidin-1-ylmethyl)-2-thienyl]-4,5- |
| | dihydro-2H-pyrazolo[4,3-c]quinoline |
| I-50: | 8-{5-[(dimethylamino)methyl]-2-thienyl}-5-ethyl-3-methyl-2,5-dihydro-4H- |
| | pyrazolo[4,3-c]quinolin-4-one |
| I-51: | 5-ethyl-3-methyl-8-(1-methyl-1H-pyrazol-4-yl)-2,5-dihydro-4H- |
| | pyrazolo[4,3-c]quinolin-4-one |
| I-52: | 5-ethyl-3-methyl-8-(1H-pyrazol-4-yl)-2,5-dihydro-4H-pyrazolo[4,3- |
| | c]quinolin-4-one |
| I-53: | 5-ethyl-3-methyl-8-[5-(pyrrolidin-1-ylmethyl)-2-thienyl]-2,5-dihydro-4H- |
| | pyrazolo[4,3-c]quinolin-4-one |
| I-54: | 5-ethyl-3-methyl-8-(3-pyrrolidin-1-ylpropyl)-2,5-dihydro-4H-pyrazolo[4,3- |
| | c]quinolin-4-one |
| I-55: | 5-ethyl-3-methyl-8-[5-(pyrrolidin-1-ylmethyl)-2-furyl]-2,5-dihydro-4H- |
| | pyrazolo[4,3-c]quinolin-4-one |
| I-56: | 8-{5-[(dimethylamino)methyl]-2-thienyl}-3-methyl-5-propyl-2,5-dihydro- |
| | 4H-pyrazolo[4,3-c]quinolin-4-one |
| I-57: | 8-{5-[(dimethylamino)methyl]-2-thienyl}-5-(2-fluoroethyl)-3-methyl-2,5- |
| | dihydro-4H-pyrazolo[4,3-c]quinolin-4-one |
| I-58: | 5-ethyl-3-methyl-8-[1-(2-pyrrolidin-1-ylethyl)-1H-pyrazol-4-yl]-2,5-dihydro- |
| | 4H-pyrazolo[4,3-c]quinolin-4-one |
| I-59: | 5-(cyclopropylmethyl)-3-methyl-8-[5-(piperidin-1-ylmethyl)-2-thienyl]-2,5- |
| | dihydro-4H-pyrazolo[4,3-c]quinolin-4-one |
| I-60: | 8-{5-[(2,5-dimethylpyrrolidin-1-yl)methyl]-2-thienyl}-5-ethyl-3-methyl-2,5- |
| | dihydro-4H-pyrazolo[4,3-c]quinolin-4-one |
| I-61: | 8-[3-(aminomethyl)phenyl]-5-ethyl-3-methyl-2,5-dihydro-4H-pyrazolo[4,3- |
| | c]quinolin-4-one |
| I-62: | 8-[4-(aminomethyl)phenyl]-5-ethyl-3-methyl-2,5-dihydro-4H-pyrazolo[4,3- |
| | c]quinolin-4-one |
| I-63: | 8-{5-[1-(2,5-dimethylpyrrolidin-1-yl)ethyl]-2-thienyl}-5-ethyl-3-methyl-2,5- |
| | dihydro-4H-pyrazolo[4,3-c]quinolin-4-one |
| I-64: | 8-[5-(aminomethyl)-2-thienyl]-5-ethyl-3-methyl-2,5-dihydro-4H- |
| | pyrazolo[4,3-c]quinolin-4-one |
| I-65: | 5-ethyl-3-methyl-8-{5-[(methylamino)methyl]-2-thienyl}-2,5-dihydro-4H- |
| | pyrazolo[4,3-c]quinolin-4-one |
| I-66: | 8-(5-amino-2-thienyl)-5-ethyl-3-methyl-2,5-dihydro-4H-pyrazolo[4,3- |
| | c]quinolin-4-one |
| I-67: | 5-ethyl-3-methyl-8-[5-(methylamino)-2-thienyl]-2,5-dihydro-4H- |
| | pyrazolo[4,3-c]quinolin-4-on |
| I-68: | 8-[5-(2-aminoethyl)-2-thienyl]-5-ethyl-3-methyl-2,5-dihydro-4H- |
| | pyrazolo[4,3-c]quinolin-4-one |
| I-69: | 8-{5-[2-(dimethylamino)ethyl]-2-thienyl}-5-ethyl-3-methyl-2,5-dihydro-4H- |
| | pyrazolo[4,3-c]quinolin-4-one |
| I-70: | 5-ethyl-3-methyl-8-{5-[2-(methylamino)ethyl]-2-thienyl}-2,5-dihydro-4H- |
| | pyrazolo[4,3-c]quinolin-4-one |
| I-71: | 5-ethyl-3-methyl-8-[5-(2-pyrrolidin-1-ylethyl)-2-thienyl]-2,5-dihydro-4H- |
| | pyrazolo[4,3-c]quinolin-4-one |
| I-72: | 5-ethyl-3-methyl-7-[5-(pyrrolidin-1-ylmethyl)-2-thienyl]-2,5-dihydro-4H- |
| | pyrazolo[4,3-c]quinolin-4-one |
| I-73: | 5-ethyl-3-methyl-8-[5-(pyrrolidin-1-ylmethyl)-2-thienyl]-2,5-dihydro-4H- |
| | pyrazolo[4,3-c]-1,8-naphthyridin-4-one |
| I-74: | 3-methyl-8-[5-(piperidin-1-ylmethyl)-2-thienyl]-5-propyl-2,5-dihydro-4H- |
| | pyrazolo[4,3-c]quinolin-4-one |
| I-75: | 8-[5-(azetidin-1-ylmethyl)-2-thienyl]-5-ethyl-3-methyl-2,5-dihydro-4H- |
| | pyrazolo[4,3-c]quinolin-4-one |
| I-76: | 8-{5-[(3,3-difluoropyrrolidin-1-yl)methyl]-2-thienyl}-5-ethyl-3-methyl-2,5- |
| | dihydro-4H-pyrazolo[4,3-c]quinolin-4-one |
| I-77: | 5-ethyl-8-{5-[(3-hydroxyazetidin-1-yl)methyl]-2-thienyl}-3-methyl-2,5- |
| | dihydro-4H-pyrazolo[4,3-c]quinolin-4-one |
| I-78: | 8-(5-{[(2S,5S)-2,5-dimethylpyrrolidin-1-yl]methyl}-2-thienyl)-5-ethyl-3- |
| | methyl-2,5-dihydro-4H-pyrazolo[4,3-c]quinolin-4-one |
| I-79: | 8-[5-(8-azabicyclo[3.2.1]oct-8-ylmethyl)-2-thienyl]-5-ethyl-3-methyl-2,5- |
| | dihydro-4H-pyrazolo[4,3-c]quinolin-4-one |
| I-80: | 1-{[5-(5-ethyl-3-methyl-4-oxo-4,5-dihydro-2H-pyrazolo[4,3-c]quinolin-8- |
| | yl)-2-thienyl]methyl}pyrrolidine-2-carboxylic acid |
| I-81: | 5-ethyl-3-methyl-8-{5-[(2,2,6,6-tetramethylpiperidin-1-yl)methyl]-2- |
| | thienyl}-2,5-dihydro-4H-pyrazolo[4,3-c]quinolin-4-one |
| I-82: | 8-{5-[2-(diethylamino)ethyl]-2-thienyl}-5-ethyl-3-methyl-2,5-dihydro-4H- |
| | pyrazolo[4,3-c]quinolin-4-one |
| I-83: | 5-ethyl-8-(5-{[(3S)-3-hydroxypyrrolidin-1-yl]methyl}-2-thienyl)-3-methyl- |
| | 2,5-dihydro-4H-pyrazolo[4,3-c]quinolin-4-one |
| I-84: | 8-[(1E)-3-(diisopropylamino)prop-1-en-1-yl]-5-ethyl-3-(2-methoxyethyl)- |
| | 2,5-dihydro-4H-pyrazolo[4,3-c]quinolin-4-one |
| I-85: | 8-[(1E)-3-(diisopropylamino)prop-1-en-1-yl]-5-ethyl-3-(2-hydroxyethyl)-2,5- |
| | dihydro-4H-pyrazolo[4,3-c]quinolin-4-one |
| I-86: | 8-[(1E)-3-(diethylamino)prop-1-en-1-yl]-3-(2-methoxyethyl)-5-propyl-2,5- |
| | dihydro-4H-pyrazolo[4,3-c]quinolin-4-one |
| I-87: | 8-[(1E)-3-(diethylamino)prop-1-en-1-yl]-3-(2-hydroxyethyl)-5-propyl-2,5- |
| | dihydro-4H-pyrazolo[4,3-c]quinolin-4-one |
| I-88: | 8-{(1E)-3-[(2S,5S)-2,5-dimethylpyrrolidin-1-yl]prop-1-en-1-yl}-5-ethyl-3-(2- |
| | methoxyethyl)-2,5-dihydro-4H-pyrazolo[4,3-c]quinolin-4-one |
| I-89: | 8-{(1E)-3-[(2S,5S)-2,5-dimethylpyrrolidin-1-yl]prop-1-en-1-yl}-5-ethyl-3-(2- |
| | hydroxyethyl)-2,5-dihydro-4H-pyrazolo[4,3-c]quinolin-4-one |

The depiction of R² in Structural Formula (I) indicates that R² is permissibly bonded to either of the nitrogen atoms in the pyrazolo or triazolo ring. Thus, Structural Formula (I) encompasses Structural Formula (XIII) and (XIV):

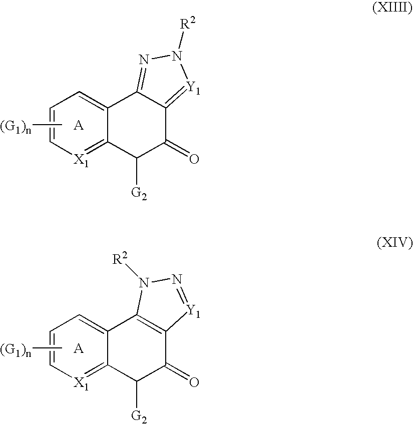

R² in Structural Formulas (I), (VI) and (VII) is —H or a group that is cleavable in vivo. The term “cleavable in vivo” means that after the Chk-1 inhibitor is administered to a subject, at least half of the cleavable groups R² groups are converted to —H before half of the administered Chk-1 inhibitor is cleared from the subject or metabolized to a form that is inactive with respect to Chk-1. A cleavable R² group can be converted to —H either by hydrolysis or enzymatically. Examples of suitable cleavable groups for R² include —S(O)₂R to form a sulfonamide, —C(O)—R to form an amide, —C(O)—OR to form a carbamate and —C(O)—NHR or —C(O)—NR₂ to form a urea, wherein R is an optionally substituted alkyl or an optionally substituted aryl group, (preferably an unsubstituted alkyl or an optionally substituted aryl group such as an optionally substituted phenyl group) or —NR₂ is a substituted or unsubstituted heteroaryl or non-aromatic heterocyclic group. Specific examples of pyrazoles with cleavable groups are shown below:

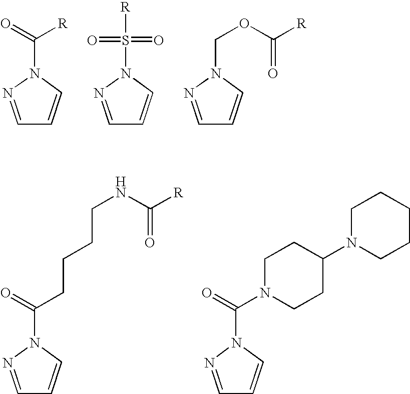

When R² represents —H, two tautomeric forms of the molecule are possible. By way of example, these two tautomeric forms are shown below for Structural Formula (I): 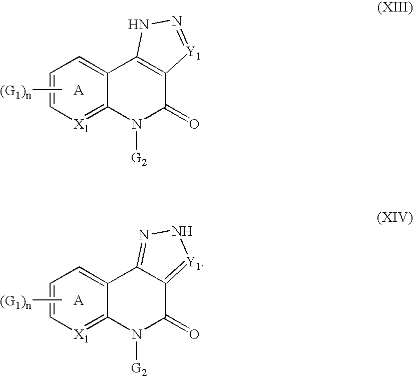
It is to be understood that both tautomeric forms are contemplated for the Chk-1 inhibitors disclosed herein.

Some of the disclosed Chk-1 inhibitors contain one or more chiral centers. The presence of chiral centers in a molecule gives rise to stereoisomers. For example, a pair of optical isomers, referred to as “enantiomers”, exist for every chiral center in a molecule; and a pair of diastereomers exist for every chiral center in a compound having two or more chiral centers.

When a disclosed Chk-1 inhibitor is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer. When a mixture is enriched in one enantiomer relative to its optical isomers, the mixture contains, for example, an enantiomeric excess of at least 50%, 75%, 90%, 95% 99% or 99.5%.

The enantiomers of the present invention may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. Where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a disclosed Chk-1 is named or depicted by structure without indicating the stereochemistry and has at least two chiral centers, it is to be understood that the name or structure encompasses a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s). When a mixture is enriched in one diastereomer or diastereomeric pair(s) relative to the other diastereomers or diastereomeric pair(s), the mixture is enriched with the depicted or referenced diastereomer or diastereomeric pair(s) relative to other diastereomers or diastereomeric pair(s) for the compound, for example, by a molar excess of at least 50%, 75%, 90%, 95% 99% or 99.5%.

The diastereoisomeric pairs may be separated by methods known to those skilled in the art, for example chromatography or crystallization, and the individual enantiomers within each pair may be separated as described above. In certain instances compounds of the present invention may be associated in isolated form with solvent or water, as in a “solvate” or “hydrate”. References to the disclosed compounds or structural formulas depicting the disclosed compounds are meant to include such solvates and hydrates.

An “aliphatic group” is non-aromatic, consists solely of carbon and hydrogen and may optionally contain one or more units of unsaturation, e.g., double and/or triple bonds. An aliphatic group may be straight-chained, branched or cyclic (i.e., “cycloaliphatic”). When straight-chained or branched, an aliphatic group typically contains between about 1 and about 12 carbon atoms, typically between about 1 and about 6 carbon atoms, more typically between about 1 and about 4 carbon atoms. When cyclic, an aliphatic group typically contains between about 3 and about 12 carbon atoms, more typically between about 3 and about 7 carbon atoms. An aliphatic group may be optionally substituted at any “substitutable carbon atom”. A “substitutable carbon atom” in an aliphatic group is a carbon in an aliphatic group that is bonded to one or more hydrogen atoms. One or more hydrogen atoms can be optionally replaced with a suitable substituent group. A “haloaliphatic group” is an aliphatic group, as defined above, substituted with one or more halogen atoms. Suitable substituents on a substitutable carbon atom of an aliphatic group are the same as those for an alkyl group.

A cycloaliphatic group can be monocyclic, fused bicyclic or bridged bicyclic. A fused bicyclic cycloaliphatic group comprises two cycloaliphic rings sharing two adjacent ring carbon atoms. A bridged bicyclic cycloaliphatic group comprises two cycloaliphic rings sharing three or four adjacent ring carbon atoms. Examples of bridged bicyclic cycloaliphatic groups include bicyclodecyl, bicyclononyl, bicyclooctyl bicycloheptanyl bicyclohexanyl and bicyclopentyl.

The term “alkyl” as used herein means saturated straight-chain, branched or cyclic hydrocarbons. When straight-chained or branched, an alkyl group is typically C₁₋₈, more typically C₁₋₆; when cyclic, an alkyl group is typically C₃₋₁₂, more typically C₃₋₇. The terms “alkyl”, “alkoxy”, “hydroxyalkyl”, “haloalkyl”, “aralkyl” “alkoxyalkyl”, “alkylamine”, “dialkyamine”, “alkylamino”, “dialkyamino” “alkoxycarbonyl” and the like, used alone or as part of a larger moiety includes both straight and branched saturated chains containing one to eight carbon atoms. The term “cycloalkyl” used alone or as part of a larger moiety shall include cyclic C₃-C₁₂ hydrocarbons which are completely saturated

The term “alkoxy” means —O-alkyl, where alkyl is as defined above.

The terms “haloalkyl” and “haloalkoxy” means alkyl or alkoxy, as the case may be, substituted with one or more halogen atoms. The term “halogen” means F, Cl, Br or I. Preferably the halogen in a haloalkyl or haloalkoxy is F.

The term “acyl group” mean —C(O)R, wherein R is an optionally substituted alkyl group or aryl group (e.g., optionally substituted phenyl). R is preferably an unsubstituted alkyl group or phenyl.

A bivalent aliphatic group is an aliphatic group in a molecule bonded to two other groups by two of its carbon atoms, each carbon atom being connected by a single covalent bond. Examples of bivalent aliphatic groups include alkylidene groups and polymethylene groups. Suitable substituents for a bivalent aliphatic group are the same as for an monovalent aliphatic group (i.e., an aliphatic group attached to another group in the molecule through a single covalent bond from one of its carbon atoms).

An “alkylene group” is represented by —[CH₂]_{z}—, wherein z is a positive integer, preferably from one to eight, more preferably from one to six. The terms “arylene”, “heterocyclene” and “carbocyclene”/“cycloalkylene” refer to aryl, non-aromatic heterocyclic or carbocyclic/cycloalkyl ring(s) in a molecule that are bonded to two other groups in the molecule through a single covalent from two of its ring atoms. Examples include phenylene [—(C₆H₄)—], thienylene [—(C₄H₂S)—], furanylene [—(C₄H₂O)—], pyrrolodinylene [—(C₄H₅N)—] and cyclohexylene [—(C₆H₁₀)—]. By way of example, the structure of 1,4-phenylene, 2,5-thienylene, 1,4 cyclohexylene and 2,5-pyrrolodinylene are shown below:

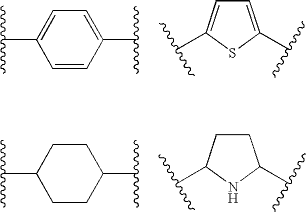

An “alkylidene group” is an alkylene group in which one or more hydrogen atoms are optionally replaced with suitable substituents. Suitable substituents are as defined below for alkyl groups. Preferred substituents include alkyl, hydroxyl, alkoxy, amine, alkylamine, dialkylamine, spiro cycloalkyl, fused cycloalkyl and non-aromatic heterocyclic group. Additional preferred substituents include oxo, halo, hydroxyalkyl, alkoxyalkyl, aminoalkyl. V₃, V₄ and V₅ are defined to be alkylidene groups. One of ordinary skill in the art will recognize that substitution of the alpha carbon atom of V₃, V₄ and V₁ (, for example, the carbon atom bonded to R¹³) with a hydroxyl, cyano or amine will result in a functional group which is not sufficiently stable for pharmaceutical use when certain values of R¹³ are selected. By way of example, when R¹³ is —OH or —CN, substitution of the alpha carbon of V₃ with —OH will result in —CH(OH)OH and —CH(OH)CN, respectively, both of which are not sufficiently stable for pharmaceutical use. Such groups are not within the scope of the present invention. Thus, when R¹³ is, —OR¹², —CN, —NR¹¹R¹², —NR¹¹CONR¹¹R¹², —NR¹¹COR¹², —NH—C(═NR¹¹)NR¹¹R¹², —N═C(NR¹¹R¹²)₂, —NR¹¹SO₂R¹², —OC(O)R¹², —NR¹¹C(O)OR¹², —O—C(O)—OR¹², —OC(O)—NR¹¹R¹², —NR¹¹CO—CH(OR¹⁸)—R¹², —NR¹¹CO—CH(NR¹⁸R¹⁸)—R¹², —NR¹¹CO—(CH₂)ₙCH(NR¹⁸R¹⁸)—R¹², —OC(O)—CH(OR¹⁸)—R¹², —OC(O)—CH(NR¹⁸R¹⁸)—R¹², —NR¹¹CO—C(R¹⁹R¹⁹)—OR¹², —NR¹¹CO—C(R¹⁹R¹⁹)—NR¹¹R¹², —OC(O)—C(R¹⁹R¹⁹)—OR¹², —OC(O)—C(R¹⁹R¹⁹)—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², or —NHC(O)NR¹¹R¹², then the alpha carbon of V₃ and V₄ is preferably unsubstituted or optionally substituted with one or two methyl groups or a spiro cycloalkyl group.

A “spiro cycloalkyl” or “spiro non-aromatic heterocyclic” group is a cycloalkyl or non-aromatic heterocyclic group which shares one ring carbon atom with a carbon atom in an alkylene group or alkyl group, wherein the carbon atom being shared in the alkyl group is not a terminal carbon atom.

The term “oxo” means a group of the formula: “═O”.

The term “heteroatom” means nitrogen, oxygen, or sulfur and includes any oxidized form of nitrogen and sulfur, and the quaternized form of any basic nitrogen. Also the term “nitrogen” includes a substitutable nitrogen of a heteroaryl or non-aromatic heterocyclic group. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or NR″ (as in N-substituted pyrrolidinyl), wherein R″ is a suitable substituent for the nitrogen atom in the ring of a non-aromatic nitrogen-containing heterocyclic group, as defined below.

The term “aromatic group” used alone or as part of a larger moiety as in “aralkyl”, “aralkoxy”, or “aryloxyalkyl”, includes carbocyclic aromatic rings and heteroaryl rings. The term “aromatic group” may be used interchangeably with the terms “aryl”, “aryl ring” “aromatic ring”, “aryl group” and “aromatic group”.

Carbocyclic aromatic ring groups have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring”, as it is used herein, is a group in which an aromatic ring is fused to one or more non-aromatic rings (cycloalkyl or heterocyclic), such as in an indanyl, phthalimidyl, naphthimidyl, phenanthridinyl, or tetrahydronaphthyl, where the radical or point of attachment is on the aromatic ring.

The term “heteroaryl”, “heteroaromatic”, “heteroaryl ring”, “heteroaryl group” and “heteroaromatic group”, used alone or as part of a larger moiety as in “heteroaralkyl” or “heteroarylalkoxy”, refers to heteroaromatic ring groups having five to fourteen members, including monocyclic heteroaromatic rings and polycyclic aromatic rings in which a monocyclic aromatic ring is fused to one or more other carbocyclic or heteroaromatic aromatic rings. Heteroaryl groups have one or more ring heteroatoms. Examples of heteroaryl groups include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl.

The term “non-aromatic heterocyclic group”, used alone or as part of a larger moiety as in “non-aromatic heterocyclylalkyl group”, refers to non-aromatic ring systems typically having five to fourteen members, preferably five to ten, in which one or more ring carbons, preferably one to four, are each replaced by a heteroatom such as N, O, or S. A “nitrogen-containing non-aromatic heterocyclic group” is a non-aromatic heterocyclic group with at least one nitrogen ring atom, and can be monocyclic, fused bicyclic or bridged bicyclic. A fused bicyclic non-aromatic heterocyclic group comprises two non-aromatic rings, one of which is nitrogen containing, that share two adjacent ring atoms. A bridged bicyclic non-aromatic heterocyclic group comprises two non-aromatic rings, one of which is nitrogen containing, that share three or four adjacent ring atoms.

Examples of non-aromatic heterocyclic groups include 3-1H-benzimidazol-2-one, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, N-azetidinyl, 1-azetidinyl, 2-azetidinyl, N-oxazolidinyl, 2-oxazolidinyl, 4-oxazolidinyl, 5-oxazolidinyl, N-morpholinyl, 2-morpholinyl, 3-morpholinyl, N-thiomorpholinyl, 2-thiomorpholinyl, 3-thiomorpholinyl, N-pyrrolidinyl, 2-pyrrolidinyl, 3-pyrrolidinyl, N-piperazinyl, 2-piperazinyl, N-piperidinyl, 2-piperidinyl, 3-piperidinyl, 4-piperidinyl, N-pyrrolidinyl, 2-pyrrolidinyl, 3-pyrrolidinyl, 4-thiazolidinyl, diazolonyl, N-substituted diazolonyl, 1-pthalimidinyl, benzoxanyl, benzopyrrolidinyl, benzopiperidinyl, benzoxolanyl, benzothiolanyl, benzothianyl, tetrahydroquinolinyl, tetrahydroisoquinolinyl, azabicyclopentyl, azabicyclohexyl, azabicycloheptyl, azabicyclooctyl, azabicyclononyl, azabicyclodecyl, diazabicyclohexyl, diazabicycloheptyl, diazabicyclooctyl, diazabicyclononyl, and diazabicyclodecyl. Also included within the scope of the term “non-aromatic heterocyclic group”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The designation “N” on N-morpholinyl, N-thiomorpholinyl, N-pyrrolidinyl, N-piperazinyl and N-piperidinyl indicates that the non-aromatic heterocyclic group is attached to the remainder of the molecule at the ring nitrogen atom.

An “aralkyl group”, “heteroaralkyl group” or “non-aromatic heterocyclylalkyl” are an alkyl group substituted with an aryl, heteroaryl or non-aromatic heterocyclic group, respectively.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen. Thus, the carbon atom bonded to R⁴ in Structural Formula (V) is not a “substitutable ring atom” within the meaning of the term, as it is used herein.

An aryl group (including, but not limited to, Ring A, and aryl groups represented by R⁷, R¹², R¹⁴, R¹⁵, R¹⁶, R¹², R^{13a}, R^{13b}, Cy, NR¹¹R¹², R⁵⁰, R⁶¹, R²⁰¹, R²⁰² and R^{x}) may contain one or more substitutable ring atoms, each bonded to a suitable substituent. Examples of suitable substituents on a substitutable ring carbon atom of an aryl group include halogen, R^{o}, —OR^{o}, —O(haloalkyl), —SR^{o}, trialkylsilyl, boronate, alkylboronate, dialkylboronate, —NO₂, —CN, —N(R′)₂, —NR′CO₂R^{o}, —NR′C(O)R^{o}, —NR′NR′C(O)R^{o}, —N(R′)C(O)N(R′)₂, —NR′NR′C(O)N(R′)₂, —NR′NR′CO₂R^{o}, —C(O)C(O)R^{o}, —C(O)CH₂C(O)R^{o}, —CO₂R^{o}, —C(O)R^{o}, —C(O)N(R^{o})₂, —OC(O)R^{o}, —OC(O)N(R^{o})₂, —S(O)₂R^{o}, —SO₂N(R′)₂, —S(O)R^{o}, —NR′SO₂N(R′)₂, —NR′SO₂R^{o}, —C(═S)N(R′)₂, —NR′—C(═NH)—N(R′)₂ and —C(═NH)—N(R′)₂ or two adjacent ring carbon atoms may be substituted with 1,2-methylene-dioxy or 1,2-ethylene-dioxy.

Each R′ is independently R^{o}, —CO₂R^{o}, —SO₂R^{o} or —C(O)R^{o} or —NR′R′ is an optionally substituted non-aromatic nitrogen-containing heterocyclic group;

Each R^{o} is independently hydrogen or an alkyl group, non-aromatic heterocyclic group or aromatic group and the alkyl, non-aromatic heterocyclic group and aromatic group represented by R^{o} is optionally substituted with one or more independently selected groups represented by R^{#}.

R^{#} is R⁺, —OR⁺, —O(haloalkyl), —SR⁺, —NO₂, —CN, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂.

R⁺ is —H, a C1-C3 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine; or —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

An alkyl or aliphatic group (including, but not limited to, groups represented by R¹, R³, R⁴, R⁵, R⁶, R⁷, R¹¹, R¹², R¹⁴, R¹⁵, R¹⁶, R¹⁸, R¹⁹, R²⁰, R⁵², R⁵³, R⁶², R⁶³, R²⁰⁰, R²⁰¹, R^{x}, V₁, V₃, V₄, V₅, V₆, T₀, G₂, W₁, and NR¹¹R¹²) or a non-aromatic heterocyclic group (including, but not limited to, non-aromatic heterocyclic groups represented by R⁷, R¹², R^{13a}, R^{13b}, R⁵⁰, R⁵¹, R⁵², R⁶¹, R²⁰¹, R²⁰², R^{x}, V₅, Cy, NR¹¹R¹², NR⁶²R⁶² and —NR¹⁸R¹⁸) may contain one or more substituents. Examples of suitable substituents for an alkyl or aliphatic group or a ring carbon of a non-aromatic heterocyclic group include those listed above for a substitutable carbon of an aryl and the following: ═O, ═S, ═NNHR*, ═NN(R*)₂, ═NNHC(O)R*, ═NNHCO₂ (alkyl), ═NNHSO₂ (alkyl), ═NR*, spiro cycloalkyl group or fused cycloalkyl group Each R* is independently selected from hydrogen, an unsubstituted alkyl group or a substituted alkyl group. Examples of substituents on the alkyl group represented by R^{*} include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl. When R¹ is substituted with cycloalkyl or phenyl the cycloalkyl and phenyl are preferably unsubstituted.

Two rings are fused when they share two adjacent ring atoms. A cycloalkyl group or non-aromatic heterocyclic group is fused to an alkyl or alkylidene group when two adjancent ring carbons from the cycloalkyl group or non-aromatic heterocyclic group are also adjacent carbon atoms in the alkyl or alkylidene group.

A “substitutable ring atom” in a non-aromatic carbocylic or nitrogen-containing non-aromatic heterocyclic group is a ring carbon or nitrogen atom that is bonded to at least one hydrogen atom. The hydrogen atom can therefore optionally be replaced with the substituent. The term “substitutable ring atom” therefore excludes ring nitrogen and carbon atoms that already have three (for nitrogen) and four (for carbon) bonds to atoms other than hydrogen.

A preferred position for substitution of a non-aromatic nitrogen-containing heterocyclic group is the nitrogen ring atom. Suitable substitutents on the nitrogen of a non-aromatic heterocyclic group or heteroaryl group include —Rˆ, —N(R)₂, —C(O)Rˆ, —CO₂Rˆ, —C(O)C(O)Rˆ, —C(O)CH₂C(O)Rˆ, —SO₂Rˆ, —SO₂N(Rˆ)₂, —C(═S)N(Rˆ)₂, —C(═NH)—N(Rˆ)₂, and —NRˆSO₂Rˆ; wherein Rˆ is hydrogen, an alkyl group, a substituted alkyl group, phenyl (Ph), substituted Ph, —O(Ph), substituted —O(Ph), CH₂(Ph), or an unsubstituted heteroaryl or heterocyclic ring. Examples of substituents on the alkyl group or the phenyl ring represented by Rˆ include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl. Preferred substituents on a substitutable nitrogen atom of a nitrogen-containing heteroaryl or nitrogen-containing non-aromatic heterocyclic group include C1-C3 alkyl, C1-C3 acyl, C1-C3 alkylsulfonyl, —OC(O)N(R′)₂, —NR′C(O)OR′, or —NR′C(O)N(R′)₂ group, where R′ is H or C1-C3 alkyl.

Non-aromatic nitrogen containing heterocyclic rings that are substituted on a ring nitrogen and attached to the remainder of the molecule at a ring carbon atom are said to be N-substituted. For example, an N-alkyl-piperidinyl group is attached to the remainder of the molecule at the two, three or four position of the piperidinyl ring and substituted at the ring ntitrogen with an alkyl group. Non-aromatic nitrogen containing heterocyclic rings such as pyrazinyl that are substituted on a ring nitrogen and attached to the remainder of the molecule at a second ring nitrogen atom are said to be N′-substituted-N-heterocycles. For example, an N′-acyl-N-pyrazinyl group is attached to the remainder of the molecule at one ring nitrogen atom and substituted at the second ring nitrogen atom with an acyl group.

Additionally, pharmaceutically acceptable salts of the compounds of the disclosed Chk-1 inhibitors are included in the present invention. For example, an acid salt of a compound containing an amine or other basic group can be obtained, by reacting the compound with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid.

Salts of compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-β-phenethylamine, dehydroabietylamine, N,N′-bisdehydroabietylamine, glucamine, N-methylglucamine, collidine, quinine, quinoline, and basic amino acid such as lysine and arginine.

The disclosed Chk-1 inhibitors are advantageously administered to inhibit Chk-1 in a subject in whom a beneficial therapeutic or prophylactic effect can be achieved by inhibiting Chk-1, i.e., a subject in need of Chk-1 inhibition. A “subject” is a mammal, preferably a human or an animal in need of veterinary treatment, e.g., companion animals (e.g., dogs, cats, and the like), farm animals (e.g., cows, sheep, pigs, horses, and the like), and laboratory animals (e.g., rats, mice, guinea pigs, and the like).

The disclosed Chk-1 inhibitors are particularly useful in therapeutic applications relating to a Chk-1-mediated disorder. As used herein, the term “Chk-1-mediated disorder” includes any disorder, disease or condition which is caused or characterized by an increase in Chk-1 expression or activity, or which requires Chk-1 activity. The term “Chk-1-mediated disorder” also includes any disorder, disease or condition in which inhibition of Chk-1 activity is beneficial.

Chk-1 inhibition can be used to achieve a beneficial therapeutic or prophylactic effect, for example, in subjects with a proliferative disorder. Non-limiting examples of proliferative disorders include chronic inflammatory proliferative disorders, e.g., psoriasis and rheumatoid arthritis; proliferative ocular disorders, e.g., diabetic retinopathy; benign proliferative disorders, e.g., hemangiomas; and cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

Non-limiting examples of solid tumors that can be treated with the disclosed Chk-1 inhibitors include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

Non-limiting examples of hematologic malignancies that can be treated with the disclosed Chk-1 inhibitors include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non—Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

The disclosed Chk-1 inhibitors are particularly useful in the treatment of cancers or cell types in which Chk-1 protein or activity is upregulated, including, without limitation, rapidly proliferating cells and drug-resistant cells (Shyjan et al., U.S. Pat. No. 6,723,498 (2004)), as well as retinoblastomas such as Rb negative or inactivated cells (Gottifredi et al., Mol. Cell. Biol., 21:1066 (2001)), or where the ARF^{p14/p19} locus has been inactivated or misregulated. The disclosed Chk-1 inhibitors also are particularly useful in the treatment of cancers or cell types in which another checkpoint pathway has been mutated or abrogated, including, without limitation, cancers or cell types in which p53 or the p53 pathway has been inactivated or abrogated.

The disclosed Chk-1 inhibitors can be administered in conjunction with other therapeutic agents, including anticancer agents. As used herein, the term “anticancer agent” refers to any agent that is administered to a subject with cancer for purposes of treating the cancer. Use of Chk-1 inhibitors for the treatment of cancer is particularly advantageous and can enhance the effectiveness of the treatment when: 1) combined with radiation therapy or chemotherapeutic agents that act by causing damage to the genetic material of cells (collectively referred to herein as “DNA damaging agents”); 2) combined with agents which are otherwise cytotoxic to cancer cells during cell division; 3) combined with agents which are proteasome inhibitors; 4) combined with agents which inhibit NF-κB (e.g., IKK inhibitors) (Bottero et al., Cancer Res., 61:7785 (2001); or 5) used with combinations of cancer drugs with which are not cytotoxic when administered alone, yet in combination produce a toxic effect. In preferred embodiments, a disclosed Chk-1 inhibitor is combined with a DNA damaging agent.

Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Agents that disrupt cell replication include: paclitaxel, docetaxel, and related analogs; vincristine, vinblastin, and related analogs; thalidomide and related analogs (e.g., CC-5013 and CC-4047); protein tyrosine kinase inhibitors (e.g., imatinib mesylate and gefitinib); antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

The disclosed Chk-1 inhibitors are also effective when used in combination with DNA-damaging anti-cancer drugs and/or radiation therapy to treat subjects with multi-drug resistant cancers. A cancer is resistant to a drug when it resumes a normal rate of tumor growth while undergoing treatment with the drug after the tumor had initially responded to the drug. A tumor “responds to a drug” when it exhibits a decrease in tumor mass or a decrease in the rate of tumor growth. The term “multi-drug resistant cancer” refers to cancer that is resistant to two or more drugs, often as many as five or more.

As such, an “effective amount” of the disclosed Chk-1 inhibitors is the quantity which inhibits Chk-1 when administered to a subject or which, when administered to a subject with cancer, slows tumor growth, ameliorates the symptoms of the disease and/or increases longevity. When used in combination with a DNA damaging agent, an effective amount of the Chk-1 inhibitor is the quantity at which a greater response is achieved when the Chk-1 inhibitor is co-administered with the DNA damaging anti-cancer drug and/or radiation therapy than is achieved when the DNA damaging anti-cancer drug and/or radiation therapy is administered alone. When used as a combination therapy, an “effective amount” of the DNA damaging agent is administered to the subject, which is a quantity that normally produces an anti-cancer effect.

A disclosed Chk-1 inhibitor can be co-administered with another therapeutic agent (e.g., DNA-damaging agent, agent that disrupts cell replication, proteasome inhibitor, NF-κB inhibitor, or other anticancer agent) as part of the same pharmaceutical composition or, alternatively, as separate pharmaceutical compositions. When administered separately, the Chk-1 inhibitor can be administered prior to, at the same time as, or following administration of the other agent, provided that the enhancing effect of the Chk-1 inhibitor is retained.

The amount of Chk-1 inhibitor, DNA damaging anti-cancer drug and radiation dose administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used anti-cancer drugs and radiation therapy are well known to the skilled person. Effective amounts of the disclosed Chk-1 inhibitors typically range between about 1 mg/mm² per day and about 10 grams/mm² per day, and preferably between 10 mg/mm² per day and about 5 grams/mm².

The Chk-1 inhibitors described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The Chk-1 inhibitor will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, Pa. (1995).

For oral administration, the Chk-1 inhibitor or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch, alginic acid; a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parental administration the disclosed Chk-1 inhibitor, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations described previously, the compounds may also be formulated as a depot preparation. Such long acting formulations may be administered by implantation, for example, subcutaneously or intramuscularly or by intramuscular injection. Thus, for example, as an emulsion in an acceptable oil, or ion exchange resins, or as sparingly soluble derivatives, for example, as sparingly soluble salts.

Preferably disclosed Chk-1 inhibitors or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient (viz., a compound of Structural Formula I, II or III or salts thereof) in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

The disclosed Chk-1 inhibitors can be prepared by a variety of procedures one of which is illustrated in scheme 1.

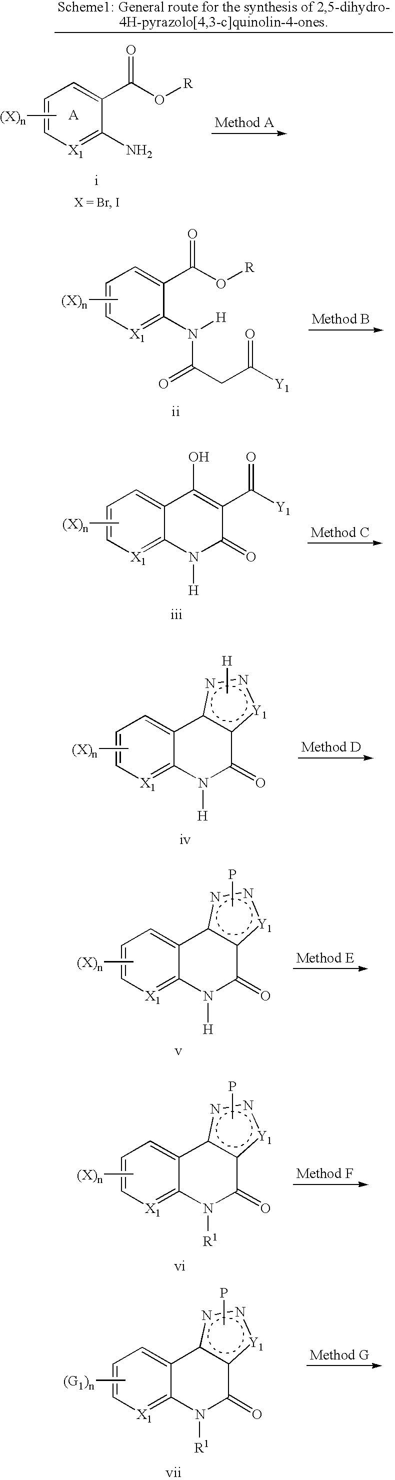

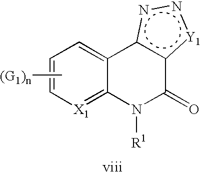

Methods for the synthesis of 2-amino benzoates of formula i are known, and exemplary synthetic procedures are described in the Examples. Conversion of i to the amido-benzoate of formula ii can be accomplished by acylation of the aniline using a suitable acyl-transfer reagent as exemplified in Method A. Compound iii may be prepared by a subsequent cyclization reaction, which may be mediated by an appropriate acid or base. Conversion of the quinolone of formula iii to the fused pyrazole of formula iv may be performed by a reaction with hydrazine or substituted hydrazine according to Method C. A suitable protecting group P may then be introduced, allowing the formation of compounds of the formula v according to Method D. Protecting groups are selected so that they are suitable for the depicted transformations and can be removed following the synthesis with little or no loss of yield. The introduction and selective removal of protecting groups are taught, e.g., in Greene and Wuts, “Protective Groups in Organic Synthesis”, John Wiley & Sons (1991) the entire contents of which are incorporated herein by reference. Compound vi may be prepared by the alkylation of the quinolone ring with a suitable alkylating reagent and may be mediated by an appropriate acid or base according to Method E. Alternatively, a substituted 2-amino benzoate, with the R¹ substituent already in place, may be used in Method A in place of compound i.

Compound vi may then undergo a cross-coupling reaction with an appropriate reagent such as a boronic acid, stannane, organozinc, amine, or amide, typically in the presence of a transition metal catalyst, according to Method F. One of ordinary skill in the art will appreciate that compounds of formula vi, wherein X is —OSO₂CF₃, may be employed in the cross-coupling reaction in place of the halides depicted in Scheme 1. Such compounds may be prepared from the compounds of formula i, wherein X is a protected hydroxyl, according to Methods A-E, followed by deprotection of the hydroxyl and conversion to the triflate.

The coupled products of formula vii can be further alkylated, acylated, oxidized, reduced, or derivatized. Alternatively, the cross-coupling reaction of Method F can be performed prior to the alkylation reaction of Method E. Those of ordinary skill in the art will recognize the feasibility of carrying out many of the transformations depicted in Scheme 1 sequentially or in a differing order of steps. The protecting group(s) may then be removed from compounds of the formula vii to afford the compounds of the formula viii, according to Method G.

### EXAMPLES

Definitions

AcOH acetic acid
aq aqueous
ATP adenosine triphosphate
BSA bovine serum albumin
Boc tert-butoxycarbonyl
DMF N,N-dimethylformamide
DCE dichloroethane
DCM dichloromethane
DMSO dimethylsulfoxide
DTT dithiothreitol
EDTA ethylenediaminetetraacetic acid
EtOAc ethyl acetate
EtOH ethanol
eq equivalents
LCMS liquid chromatography mass spectrum
MeOH methanol
MHz megahertz
MTT methylthiazoletetrazolium
rt room temperature
Rₜ retention time
XTT 2,3-bis(2-methoxy-4-nitro-5-sulfophenyl)-2H-tetrazolium-5-carboxanilide inner salt
WST (4-[3-(4-iodophenyl)-2-(4-nitrophenyl)-2H-5-tetrazolio]-1,3-benzene disulfonate sodium salt
PKA cAMP-dependent protein kinase
TBTU O-Benzotriazol-1-yl-N,N,N′,N′-tetramethyluronium tetrafluoroborate
TEA triethylamine
THF tetrahydrofuran
h hours
min minutes
m/z mass to charge
MS mass spectrum
HRMS high resolution mass spectrum
NMR nuclear magnetic resonance
   Analytical Methods

LCMS: compounds were analysed on a Phenomenex Luna column (C18, 50×4.6 mm, 5 um) eluted with 5% acetonitrile/water/0.1% formic acid (mobile phase A) and 100% acetonitrile/0.1% formic acid (mobile phase B) with a flow rate of 1.5 ml/min. The 5 min cycle consisted of a gradient of 100% A to 100% B in 3.5 min; 100% B for 1 min; 100% B to 100% A in 0.1 min; then re-equilibration with mobile phase A for 0.49 min.

NMR: proton spectra were recorded on a Bruker 300 or 400 MHz ultrashield spectrometer. Chemical shifts are reported relative to methanol (δ 3.31), dimethyl sulfoxide (δ 2.50), or chloroform (δ 7.26).

### Example 1

### Preparation of 5-Ethyl-3-methyl-8-(3-pyrrolidin-1-ylprop-1-yn-1-yl)-2,5-dihydro-4H-pyrazolo[4,3-c]quinolin-4-one

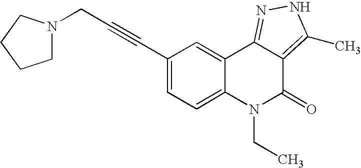

### Step 1, Preparation of 5-Iodoisatoic Anhydride

To a solution of 2-amino-5-iodobenzoic acid (50.09 g, 190.4 mmol) in 800 mL anhydrous THF at rt was added triphosgene (19.1 g, 64.4 mmol). The solution was stirred at rt for 6 h, then stored at 0° C. for 16 h. The precipitate was filtered and washed with diethyl ether to give 40.32 g product. The filtrate was then concentrated and the residue was triturated with THF/ether (1:1) then filtered and washed with ether to give and additional 9.91 g product. The overall yield was 50.23 g.

### Step 2, Preparation of Methyl 2-amino-5-iodobenzoate

To a suspension of 5-iodoisatoic anhydride (50.23 g, 173.8 mmol) in 800 mL anhydrous methanol at rt was added 4-dimethylaminopyridine (1.97 g, 16.2 mmol).

The mixture was then stirred at 80° C. for 4 h, then cooled to rt and the solvent was evaporated in vacuo. The residue was partitioned between EtOAc and 0.1 N HCl. The layers were separated and the organic phase was then washed with 0.1 N HCl (2×), brine, then dried over sodium sulfate and concentrated in vacuo to give 47.06 g product as an off-white solid.

### Step 3, Preparation of Methyl 2-(acetoacetylamino)-5-iodobenzoate

A solution of methyl 2-amino-5-iodobenzoate (17.9 g, 64.6 mmol) and methylacetoacetate (7.0 mL, 64.6 mmol) in toluene (250 mL) was heated to reflux using a Soxhlet extractor filled with 3 angstrom molecular sieves. After 24 h, the molecular sieves were replaced, more methylacetoacetate (3.75 mL, 32.3 mmol) was added, and the solution was refluxed 2 days. Concentration in vacuo and wash with diethyl ether afforded 24.7 g (76%) of methyl 2-(acetoacetylamino)-5-iodobenzoate as an off-white powder. LCMS: Rₜ=1.70 min, [MH⁺362.0].

### Step 4, Preparation of 3-Acetyl-4-hydroxy-6-iodoquinolin-2(1H)-one

To a suspension of methyl 2-(acetoacetylamino)-5-iodobenzoate (2.3 g, 6.37 mmol) in CH₃OH (64 mL) was added NaOCH₃ solution in CH₃OH (1.09 mL, 4.78 mmol) dropwise via syringe. The mixture was heated to 70° C. for 3 h then cooled to rt and diluted with 1.0 N HCl solution (50 mL) and filtered. The resulting solid was washed with H₂O (2×) and Et₂O (2×) and dried under high vacuum. A 91% yield of 3-acetyl-4-hydroxy-6-iodoquinolin-2(1H)-one was isolated as a white solid. LCMS: Rₜ=1.93 min, [MH⁺ 330.0].

### Step 5, Preparation of 8-Iodo-3-methyl-2,5-dihydro-4H-pyrazolo[4,3-c]quinolin-4-one

To a suspension of 3-acetyl-4-hydroxy-6-iodoquinolin-2(1H)-one (13.2 g, 40.0 mmol) in DMF (200 mL) was added hydrazine hydrate (5.8 mL, 120 mmol) and the mixture was heated to reflux for 3 h. The solution was cooled to rt before it was carefully quenched with 1.0 N HCl solution (100 mL), stirred for 1 h and filtered. The filtered material was washed with H₂O (2×) and Et₂O (2×) before being dried under high vacuum to afford 9.45 g (73%) of 8-iodo-3-methyl-2,5-dihydro-4H-pyrazolo[4,3-c]quinolin-4-one as a white solid. LCMS: Rₜ=1.37 min, [MH⁺ 326.0].

### Step 6, Preparation of 8-Iodo-3-methyl-2-(tetrahydro-2H-pyran-2-yl)-2,5-dihydro-4H-pyrazolo[4,3-c]quinolin-4-one

A mixture of 8-iodo-3-methyl-2,5-dihydro-4H-pyrazolo[4,3-c]quinolin-4-one (9.9 g, 30.6 mmol), 3,4-dihydro-2H-pyran (11.0 mL, 122.3 mmol) and p-toluenesulfonic acid (0.6 g, 3.06 mmol) was heated to 90° C. for 18 h. Dilution of the mixture with Et₂O followed by filtration afforded 9.0 g (72%) of 8-iodo-3-methyl-2-(tetrahydro-2H-pyran-2-yl)-2,5-dihydro-4H-pyrazolo[4,3-c]quinolin-4-one as a white powder. LCMS: Rₜ=1.81 min, [MH⁺ 410.0].

### Step 7, Preparation of 5-Ethyl-8-iodo-3-methyl-2-(tetrahydro-2H-pyran-2-yl)-2,5-dihydro-4H-pyrazolo[4,3-c]quinolin-4-one

A mixture of 8-iodo-3-methyl-2-(tetrahydro-2H-pyran-2-yl)-2,5-dihydro-4H-pyrazolo[4,3-c]quinolin-4-one (5.0 g, 12.2 mmol) and Cs₂CO₃ (19.9 g, 61.1 mmol) in DMF (122 mL) was stirred 10 min before ethyliodide (2.47 mL, 30.6 mmol) in DMF (5 mL) was added. The reaction mixture was heated to 90° C. and stirred for 1 h then cooled to rt. Dilution of the mixture with cold H₂O followed by filtration resulted in a white solid. The crude material was purified by crystallization in EtOAc (100 mL) and hexane (200 mL) at 0° C. for 12 h. Crystals were filtered and dried to afford 4.38 g (83%) of 5-ethyl-8-iodo-3-methyl-2-(tetrahydro-2H-pyran-2-yl)-2,5-dihydro-4H-pyrazolo[4,3-c]quinolin-4-one as a white solid. LCMS: Rₜ=2.20 min, [MH⁺ 438.3].

### Step 8, Preparation of 5-Ethyl-3-methyl-8-(3-pyrrolidin-1-ylprop-1-yn-1-yl)-2-(tetrahydro-2H-pyran-2-yl)-2,5-dihydro-4H-pyrazolo[4,3-c]quinolin-4-one

To a solution of 5-ethyl-8-iodo-3-methyl-2-(tetrahydro-2H-pyran-2-yl)-2,5-dihydro-4H-pyrazolo[4,3-c]quinolin-4-one (1.5 g, 3.43 mmol) in 35 mL DMF at rt was added dichlorobis(triphenylphosphine)palladium (84 mg, 0.12 mmol), copper iodide (52 mg, 0.274 mmol), and triethylamine (1.91 mL, 13.72 mmol). The solution was degassed, backfilled with Ar, and stirred at rt for 1 h. 1-Prop-2-yn-1-ylpyrrolidine (0.749 mL, 6.86 mmol) was then added and the solution was stirred at 60° C. for 16 h (for some alkynes the reaction was carried out at rt). The solution was then allowed to cool to rt, and was diluted with EtOAc and water. The organic phase was washed with water followed by brine, dried over sodium sulfate and the concentrated in vacuo. The residue was purified by silica gel chromatography (10-50% ethyl acetate in hexanes) to give 1.28 g product as a white solid (89%). LCMS: [MH⁺419.3].

### Step 9, Preparation of 5-Ethyl-3-methyl-8-(3-pyrrolidin-1-ylprop-1-yn-1-yl)-2,5-dihydro-4H-pyrazolo[4,3-c]quinolin-4-one

To a solution of 5-ethyl-3-methyl-8-(3-pyrrolidin-1-ylprop-1-yn-1-yl)-2-(tetrahydro-2H-pyran-2-yl)-2,5-dihydro-4H-pyrazolo[4,3-c]quinolin-4-one (1.28 g, 3.05 mmol) in CH₃OH (100 mL) was added concentrated HCl (1.0 mL) and the reaction was stirred for 4 h. Concentration in vacuo afforded 1.32 g of 5-ethyl-3-methyl-8-(3-pyrrolidin-1-ylprop-1-yn-1-yl)-2,5-dihydro-4H-pyrazolo[4,3-c]quinolin-4-one as a yellow solid. ¹H NMR (400 MHz, CD₃OD) δ 8.29 (d, 1 H), 7.72 (dd, 1 H), 7.61 (d, 1H), 4.43 (s, 2 H), 4.37 (q, 2 H), 3.81-3.72 (m, 2 H), 3.41-3.33 (m, 2 H), 2.70 (s, 3 H), 2.31-2.05 (m, 4 H), 4.32 (t, 3 H). LCMS: Rₜ=0.180 min, [MH⁺335.2].

### Example 2

### Preparation of 3-Methyl-8-(3-pyrrolidin-1-ylprop-1-yn-1-yl)-5-(2,2,2-trifluoroethyl)-2,5-dihydro-4H-pyrazolo[4,3-c]quinolin-4-one

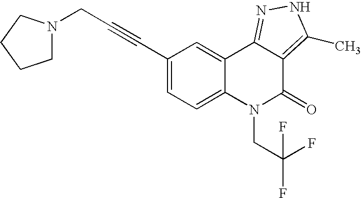

The title compound was prepared using analogous procedures as outlined in Example 1. ¹H NMR (300 MHz, DMSO-d₆) δ 8.40 (d, 1 H), 7.8-7.6 (m, 2 H), 5.30-5.10 (m, 2 H), 4.90 (s, 2 H), 3.60-3.39 (m, 2 H), 3.30 (s, 3 H), 2.60-2.55 (m, 2 H), 2.10-1.85 (m, 4 H). LCMS: Rₜ=1.07 min, [MH⁺389.2].

### Example 3

### Preparation of 3-Methyl-5-propyl-8-(3-pyrrolidin-1-ylprop-1-yn-1-yl)-2,5-dihydro-4H-pyrazolo[4,3-c]quinolin-4-one

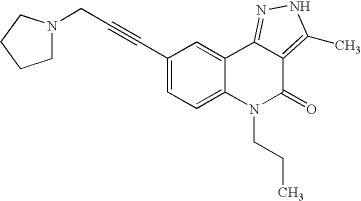

The title compound was prepared using analogous procedures as outlined in Example 1. ¹H NMR (400 MHz, DMSO-d₆) δ 7.92 (s, 1 H), 7.25 (d, 1 H), 7.17 (d, 1 H), 3.99 (d, 2 H), 3.77 (app t, 2 H), 3.20-3.11 (m, 2 H), 2.80-2.78 (m, 2 H), 2.15 (s, 3 H), 1.70-1.45 (m, 4 H), 1.25-1.15 (m, 2 H), 0.55 (t, 3 H). LCMS: Rₜ=1.12 min, [MH⁺349.4].

### Example 4

### Preparation of 5-(3-Hydroxypropyl)-3-methyl-8-(3-pyrrolidin-1-ylprop-1-yn-1-yl)-2,5-dihydro-4H-pyrazolo[4,3-c]quinolin-4-one

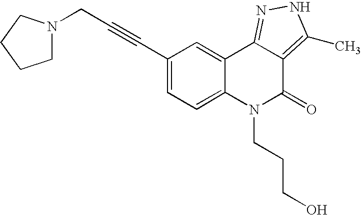

The title compound was prepared using analogous procedures as outlined in Example 1. ¹H NMR (400 MHz, DMSO-d₆) δ 7.32 (s, 1 H), 6.77-6.75 (m, 2 H), 3.57 (app t, 2 H), 2.90 (s, 2 H), 2.86 (t, 2 H), 2.02-1.98 (m, 4 H), 1.84 (s, 3 H), 1.12-1.07 (m, 6 H). LCMS: Rₜ=0.92 min, [MH⁺365.4].

### Example 5

### Preparation of 5-(2-Hydroxyethyl)-3-methyl-8-(3-pyrrolidin-1-ylprop-1-yn-1-yl)-2,5-dihydro-4H-pyrazolo[4,3-c]quinolin-4-one

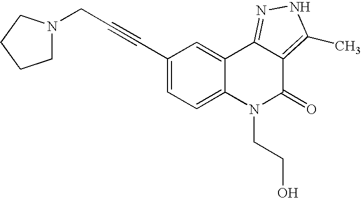

The title compound was prepared using analogous procedures as outlined in Example 1. ¹H NMR (400 MHz, DMSO-d₆) δ 13.69 (br s, 1 H), 8.15 (d, 1 H), 7.66-7.50 (m, 2 H), 4.87 (t, 1 H), 4.36-4.26 (m, 2 H), 3.66-3.61 (m, 4 H), 2.67-2.53 (m, 4 H), 1.78-1.71 (m, 4 H). LCMS: Rₜ=0.82 min, [MH⁺351.5].

### Example 6

### Preparation of 5-Isobutyl-3-methyl-8-(3-pyrrolidin-1-ylprop-1-yn-1-yl)-2,5-dihydro-4H-pyrazolo[4,3-c]quinolin-4-one

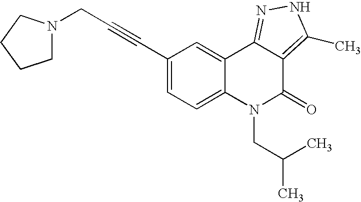

The title compound was prepared using analogous procedures as outlined in Example 1. ¹H NMR (400 MHz, DMSO-d₆) δ 10.95 (br s, 1 H), 8.27 (s, 1 H), 7.72-7.52 (m, 2 H), 4.42 (d, 2 H), 4.16-4.10 (m, 2 H), 3.65-3.55 (m, 3 H), 3.24-3.15 (m, 2 H), 2.59 (s, 3 H), 2.15-1.90 (m, 4 H), 0.89 (d, 6 H). LCMS: Rₜ=1.15 min, [MH⁺363.5].

### Example 7

### Preparation of 8-(3-Amino-3-methylbut-1-yn-1-yl)-5-ethyl-3-methyl-2,5-dihydro-4H-pyrazolo[4,3-c]quinolin-4-one

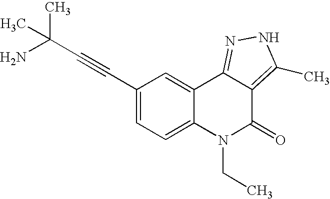

The title compound was prepared using analogous procedures as outlined in Example 1. ¹H NMR (400 MHz, CD₃OD) δ 8.25 (d, 1 H), 7.66 (dd, 1 H), 7.60 (d, 1 H), 4.38 (q, 2 H), 2.70 (s, 3 H), 1.77 (s, 6 H), 1.33 (t, 3 H). LCMS: Rₜ=0.98 min, [M2H⁺310.2].

### Example 8

### Preparation of 5-Ethyl-3-(2-methoxyethyl)-8-(3-pyrrolidin-1-ylprop-1-yn-1-yl)-2,5-dihydro-4H-pyrazolo[4,3-c]quinolin-4-one

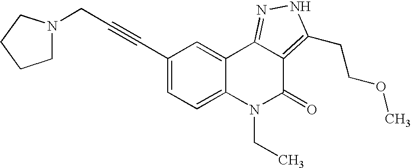

The title compound was prepared using analogous procedures as outlined in Example 1. ¹H NMR (400 MHz, CD₃OD) δ 8.31 (s, 1 H), 7.72 (d, 1 H), 7.63 (d, 1 H), 4.42-4.36 (m, 4 H), 3.81 (t, 2 H), 3.56-3.47 (m, 4 H), 3.40-3.35 (m, 5 H), 2.19-2.14 (m, 4 H), 1.34 (t, 3 H). LCMS: Rₜ=0.90 min, [MH⁺379.3].

### Example 9

### Preparation of 3-Methyl-8-(3-pyrrolidin-1-ylprop-1-yn-1-yl)-2,5-dihydro-4H-pyrazolo[4,3-c]quinolin-4-one

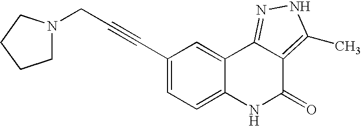

The title compound was prepared using analogous procedures as outlined in Example 1. ¹H NMR (400 MHz, CD₃OD) δ 8.15 (s, 1 H), 7.55 (d, 1 H), 7.35 (d, 1 H), 2.68 (s, 2 H), 2.82-2.76 (m, 4 H), 2.71 (s, 3 H), 1.95-1.82 (m, 4 H). LCMS: Rₜ=1.14 min, [MH⁺307.4].

### Example 10

### Preparation of 3,5-Dimethyl-8-(3-pyrrolidin-1-ylprop-1-yn-1-yl)-2,5-dihydro-4H-pyrazolo[4,3-c]quinolin-4-one

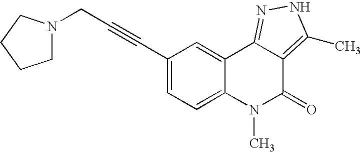

The title compound was prepared using analogous procedures as outlined in Example 1. ¹H NMR (400 MHz, CD₃OD) δ 8.25 (s, 1 H), 7.70 (d, 1 H), 7.59 (d, 1 H), 4.30 (s, 2 H), 3.70 (s, 3 H), 3.52-3.39 (m, 4 H), 2.68 (s, 3 H), 2.18-2.10 (m, 4 H). LCMS: Rₜ=0.85 min, [MH⁺321.4].

### Example 11

### Preparation of 8-[3-(Dimethylamino)prop-1-yn-1-yl]-5-ethyl-3-methyl-2,5-dihydro-4H-pyrazolo[4,3-c]quinolin-4-one

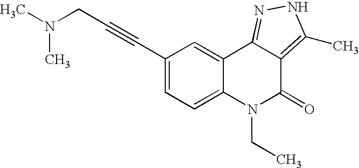

### Step 1, Preparation of 5-Ethyl-8-(3-hydroxyprop-1-yn-1-yl)-3-methyl-2-(tetrahydro-2H-pyran-2-yl)-2,5-dihydro-4H-pyrazolo[4,3-c]quinolin-4-one

To a solution of 5-ethyl-8-iodo-3-methyl-2-(tetrahydro-2H-pyran-2-yl)-2,5-dihydro-4H-pyrazolo[4,3-c]quinolin-4-one (as prepared in example 1, 2.0 g, 4.57 mmol) in 45 mL DMF at rt was added dichlorobis(triphenylphosphine)palladium (112 mg, 0.159 mmol), copper iodide (69 mg, 0.366 mmol), and triethylamine (2.54 mL, 18.3 mmol). The solution was degassed with Ar, and stirred at rt for 1 h. Prop-2-yn-1-ol (0.533 mL, 9.15 mmol) was then added and the solution was stirred at 60° C. for 16 h (for some alkynes the reaction was carried out at rt). The solution was then allowed to cool to rt, and was diluted with EtOAc and water. The organic phase was washed with water followed by brine, dried over sodium sulfate and the concentrated in vacuo. The residue was purified by silica gel chromatography (0-100% ethyl acetate in hexanes) to give 1.5 g product as a white solid (89%). LCMS: [MH⁺366.4].

### Step 2, Preparation of 3-[5-Ethyl-3-methyl-4-oxo-2-(tetrahydro-2H-pyran-2-yl)-4,5-dihydro-2H-pyrazolo[4,3-c]quinolin-8-yl]prop-2-ynal

To a solution of 5-ethyl-8-(3-hydroxyprop-1-yn-1-yl)-3-methyl-2-(tetrahydro-2H-pyran-2-yl)-2,5-dihydro-4H-pyrazolo[4,3-c]quinolin-4-one (100 mg, 0.274 mmol) in DCM (3.0 mL) was added Dess-Martin periodinane (232 mg, 0.548 mmol). The reaction was stirred 1 h, diluted with DCM and washed with sodium thiosulfate (saturated aq solution), sodium bicarbonate, and brine. The solution was dried over magnesium sulfate, filtered, and concentrated. The crude residue was taken on to the next step with no further purification.

### Step 3, Preparation of 8-[3-(Dimethylamino)prop-1-yn-1-yl]-5-ethyl-3-methyl-2-(tetrahydro-2H-pyran-2-yl)-2,5-dihydro-4H-pyrazolo[4,3-c]quinolin-4-one

To a solution of 3-[5-ethyl-3-methyl-4-oxo-2-(tetrahydro-2H-pyran-2-yl)-4,5-dihydro-2H-pyrazolo[4,3-c]quinolin-8-yl]prop-2-ynal (0.274 mmol) in DCE (3 mL) was added sodium triacetoxyborohydride (87 mg, 0.411 mmol) and dimethylamine (0.137 mL, 0.274 mmol). The reaction was stirred at 60° C. for 2 h, cooled, and diluted with EtOAc. The resulting solution was washed (sodium bicarbonate, brine), dried (magnesium sulfate), filtered, and concentrated. The crude residue was then purified by flash chromatography (gradient elution: 0-5% MeOH in DCM) to afford 73 mg as a white solid. LCMS: [MH⁺393.1].

Acidic deprotection as in example 1, step 9 provided the title compound. ¹H NMR (400 MHz, CD₃OD) δ 8.22 (s, 1 H), 7.66 (d, 1 H), 7.58 (d, 1 H), 4.38 (q, 2 H), 4.03 (s, 2 H), 2.80 (s, 6 H), 2.68 (s, 3 H), 1.35 (t, 3 H). LCMS: Rₜ=0.86 min, [MH⁺309.0].

### Example 12

### Preparation of 5-(2-Fluoroethyl)-3-methyl-8-(3-pyrrolidin-1-ylprop-1-yn-1-yl)-2,5-dihydro-4H-pyrazolo[4,3-c]quinolin-4-one

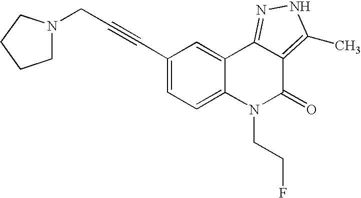

The title compound was prepared using analogous procedures as outlined in Example 1. ¹H NMR (400 MHz, CD₃OD) δ 8.30 (s, 1 H), 7.69 (s, 2 H), 4.88-4.62 (m, 4 H), 4.45 (s, 2 H), 3.82-3.71 (m, 2 H), 3.45-3.33 (m, 2 H), 2.70 (s, 3 H), 2.32-2.05 (m, 4 H). LCMS: Rₜ=0.98 min, [MH⁺353.4].

### Example 13

### Preparation of 5-Ethyl-3-methyl-8-(3-piperidin-1-ylprop-1-yn-1-yl)-2,5-dihydro-4H-pyrazolo[4,3-c]quinolin-4-one

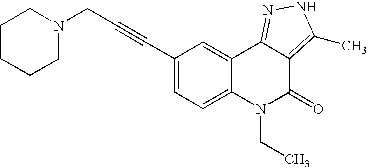

The title compound was prepared using analogous procedures as outlined in Example 11. ¹H NMR (400 MHz, CD₃OD) δ 8.30 (s, 1 H), 7.72 (d, 1 H), 7.60 (d, 1 H), 4.38 (q, 2 H), 4.35 (s, 2 H), 3.75 (d, 2 H), 3.15 (app t, 2 H), 2.70 (s, 3 H), 2.05 (d, 2 H), 1.90-1.52 (m, 4 H), 1.32 (t, 3 H). LCMS: Rₜ=0.91 min, [MH⁺349.1].

### Example 14

### Preparation of 5-Ethyl-3-methyl-8-[3-(4-methylpiperazin-1-yl)prop-1-yn-1-yl]-2,5-dihydro-4H-pyrazolo[4,3-c]quinolin-4-one

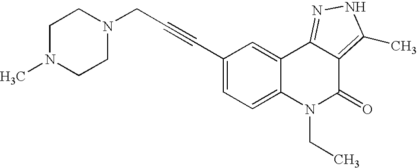

The title compound was prepared using analogous procedures as outlined in Example 11. ¹H NMR (400 MHz, CD₃OD) δ 8.32 (s, 1 H), 7.77 (d, 1 H), 7.60 (d, 1 H), 4.45 (s, 2 H), 4.39 (q, 2 H), 3.95-3.50 (m, 8 H), 3.04 (s, 3 H), 2.70 (s, 3 H), 1.32 (t, 3 H). LCMS: Rₜ=0.88 min, [MH⁺364.1].

### Example 15

### Preparation of 5-Ethyl-3-methyl-8-(3-morpholin-4-ylprop-1-yn-1-yl)-2,5-dihydro-4H-pyrazolo[4,3-c]quinolin-4-one

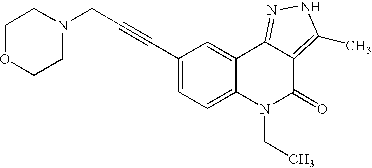

The title compound was prepared using analogous procedures as outlined in Example 11. ¹H NMR (400 MHz, CD₃OD) δ 8.29 (s, 1 H), 7.76 (d, 1 H), 7.61 (d, 1 H), 4.43 (s, 2 H), 4.38 (q, 2 H), 4.16 (d, 2 H), 3.86 (app t, 2 H), 3.70 (d, 2 H), 3.37 (d, 2 H), 2.70 (s, 3 H), 1.31 (t, 3 H). LCMS: Rₜ=0.87 min, [MH⁺351.2].

### Example 16

### Preparation of 8-{3-[(2R,5S)-2,5-Dimethylpyrrolidin-1-yl]prop-1-yn-1-yl}-5-ethyl-3-methyl-2,5-dihydro-4H-pyrazolo[4,3-c]quinolin-4-one

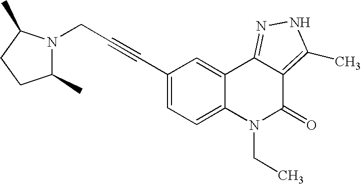

The title compound was prepared using analogous procedures as outlined in Example 11. ¹H NMR (400 MHz, CD₃OD) δ 8.16 (s, 1 H), 7.62-7.50 (m, 2 H), 4.37 (q, 2 H), 3.82 (s, 2 H), 2.96-2.92 (m, 2 H), 2.65 (s, 3 H), 1.95-1.92 (m, 2 H), 1.48-1.39 (m, 2 H), 1.33 (t, 3 H), 1.28 (d, 6 H). LCMS: Rₜ=0.99 min, [MH⁺363.5].

### Example 17

### Preparation of 8-{3-[(2S,5S)-2,5-Dimethylpyrrolidin-1-yl]prop-1-yn-1-yl}-5-ethyl-3-methyl-2,5-dihydro-4H-pyrazolo[4,3-c]quinolin-4-one (Racemic Mixture of Enantiomers)

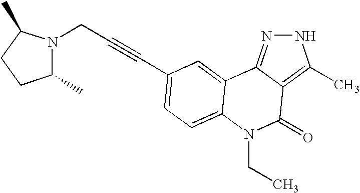

The title compound was prepared using analogous procedures as outlined in Example 11. ¹H NMR (400 MHz, CD₃OD) δ 8.16 (s, 1 H), 7.62-7.51 (m, 2 H), 4.36 (q, 2 H), 3.91-3.51 (abq, 2 H), 2.67 (s, 3 H), 2.18-2.05 (m, 2 H), 1.57-1.45 (m 2 H), 1.31 (t, 3 H), 1.16 (d, 6 H). LCMS: Rₜ=1.00 min, [MH⁺363.5].

### Example 18

### Preparation of 8-[3-(Diethylamino)prop-1-yn-1-yl]-5-ethyl-3-methyl-2,5-dihydro-4H-pyrazolo[4,3-c]quinolin-4-one

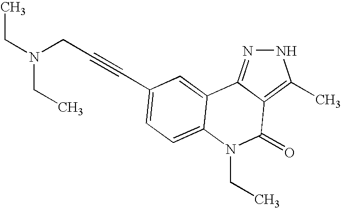

The title compound was prepared using analogous procedures as outlined in Example 11. ¹H NMR (400 MHz, CD₃OD) δ 8.30 (s, 1 H), 7.75-7.55 (m, 2 H), 4.43 (s, 2 H), 4.39 (q, 2 H), 3.45 (q, 4 H), 2.69 (s, 3 H), 1.43 (t, 6 H), 1.32 (t, 3 H). LCMS: Rₜ=0.95 min, [MH⁺337.2].

### Example 19

### Preparation of 5-(Cyclopropylmethyl)-3-methyl-8-(3-pyrrolidin-1-ylprop-1-yn-1-yl)-2,5-dihydro-4H-pyrazolo[4,3-c]quinolin-4-one

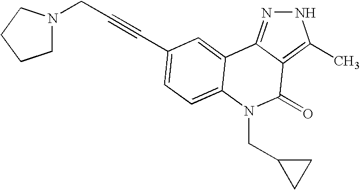

The title compound was prepared using analogous procedures as outlined in Example 1. ¹H NMR (300 MHz, CD₃OD) δ 8.27 (s, 1 H), 7.66-7.71 (m, 2 H), 4.43 (s, 2 H), 4.25 (d, 2 H), 3.73-3.80 (m, 2 H), 3.31-3.40 (m, 2 H), 2.67 (s, 3 H), 2.07-2.28 (m, 4 H), 1.24-1.32 (m, 1 H), 0.51-0.53 (m, 4 H). LCMS: Rₜ=0.95 min, [MH⁺361.0].

### Example 20

### Preparation of 5-(2-Methoxyethyl)-3-methyl-8-(3-pyrrolidin-1-ylprop-1-yn-1-yl)-2,5-dihydro-4H-pyrazolo[4,3-c]quinolin-4-one

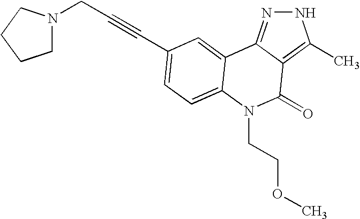

The title compound was prepared using analogous procedures as outlined in Example 1. ¹H NMR (400 MHz, CDCl₃) δ 8.65 (br s, 1 H), 7.47-7.53 (m, 2 H), 4.42-4.47 (m, 4 H), 3.95-4.00 (m, 2 H), 3.70-3.73 (m, 2 H), 3.30-3.38 (m, 5 H), 2.89 (s, 3 H), 2.25-2.30 (m, 4 H). LCMS: Rₜ=0.86 min, [MH⁺365.0].

### Example 21

### Preparation of 5-(2,2-Difluoroethyl)-3-methyl-8-(3-pyrrolidin-1-ylprop-1-yn-1-yl)-2,5-dihydro-4H-pyrazolo[4,3-c]quinolin-4-one

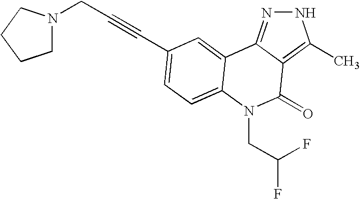

The title compound was prepared using analogous procedures as outlined in Example 1. ¹H NMR (400 MHz, DMSO-d₆) δ 8.31 (d, 1 H), 7.64-7.81 (m, 2 H), 6.06-6.34 (m, 1 H), 4.72-4.83 (m, 2 H), 4.42 (s, 2 H), 3.30-3.67 (m, 4 H), 2.70 (s, 3 H), 2.11-2.77 (m, 4 H). LCMS: Rₜ=0.98 min, [MH⁺371.0].

### Example 22

### Preparation of 5-Ethyl-3-methyl-8-[(1E)-3-pyrrolidin-1-ylprop-1-en-1-yl]-2,5-dihydro-4H-pyrazolo[4,3-c]quinolin-4-one

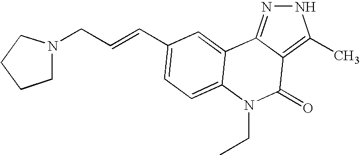

### Step 1, Preparation of Methyl (2E)-3-[5-ethyl-3-methyl-4-oxo-2-(tetrahydro-2H-pyran-2-yl)-4,5-dihydro-2H-pyrazolo[4,3-c]quinolin-8-yl]acrylate

To a suspension of K₂CO₃ (2.37 g, 17.16 mmol) and tetra-n-butylammonium chloride (1.91 g, 6.86 mmol) in DMF (30 mL) was added H₂O (3 mL) and mixture was stirred for 20 min. To the suspension were added triphenylphosphine (0.18 g, 0.686 mmol), 5-ethyl-8-iodo-3-methyl-2-(tetrahydro-2H-pyran-2-yl)-2,5-dihydro-4H-pyrazolo[4,3-c]quinolin-4-one (as prepared in Example 1, 3.0 g, 6.68 mmol) and methyl acrylate (1.24 mL, 13.37 mmol). DMF (50 mL) and H₂O (5 mL) were added to the suspension and the mixture was stirred for 15 min before palladium acetate (0.077 g, 0.343 mmol) was added and reaction mixture was heated to 50° C. for 2 h. Methyl acrylate (0.62 mL, 6.86 mmol) was added and mixture was stirred 12 h at 75° C. The reaction mixture was cooled to rt and cold H₂O was added followed by filtration affording 1.6 g (59%) of desired product methyl (2E)-3-[5-ethyl-3-methyl-4-oxo-2-(tetrahydro-2H-pyran-2-yl)-4,5-dihydro-2H-pyrazolo[4,3-c]quinolin-8-yl]acrylate. LCMS: Rₜ=1.89 min, [MH⁺ 396.2].

### Step 2, Preparation of 5-Ethyl-8-[(1E)-3-hydroxyprop-1-en-1-yl]-3-methyl-2-(tetrahydro-2H-pyran-2-yl)-2,5-dihydro-4H-pyrazolo[4,3-c]quinolin-4-one

To a −78° C. suspension of methyl (2E)-3-[5-ethyl-3-methyl-4-oxo-2-(tetrahydro-2H-pyran-2-yl)-4,5-dihydro-2H-pyrazolo[4,3-c]quinolin-8-yl]acrylate (0.25 g, 0.633 mmol) in THF (4 mL) was added lithium aluminum hydride (0.11 g, 1.27 mmol). The reaction mixture was stirred for 45 min, then quenched with H₂O (1 eq), 15% aq NaOH (1 eq), and H₂O (3 eq) all with vigorous stirring. Surplus of MgSO₄ was added and the mixture was filtered and washed with DCM. The crude material was purified by silica gel flash chromatography (0-40% EtOAc in hexanes) to afford 0.14 g (60%) of desired product 5-ethyl-8-[(1E)-3-hydroxyprop-1-en-1-yl]-3-methyl-2-(tetrahydro-2H-pyran-2-yl)-2,5-dihydro-4H-pyrazolo[4,3-c]quinolin-4-one. LCMS: Rₜ=1.49 min, [MH⁺ 368.2].

### Step 3, Preparation of (2E)-3-[5-Ethyl-3-methyl-4-oxo-2-(tetrahydro-2H-pyran-2-yl)-4,5-dihydro-2H-pyrazolo[4,3-c]quinolin-8-yl]acrylaldehyde

To a solution of 5-ethyl-8-[(1E)-3-hydroxyprop-1-en-1-yl]-3-methyl-2-(tetrahydro-2H-pyran-2-yl)-2,5-dihydro-4H-pyrazolo[4,3-c]quinolin-4-one (0.088 g, 0.240 mmol) in DCM (5 mL) was added Dess-Martin periodinane (0.203 g, 0.480 mmol). The mixture was stirred 4 h at rt and poured into a saturated aq sodium thiosulfate solution. The organic phase was separated and washed with saturated aq NaHCO₃ solution (50 mL) and water (2×50 mL). The crude material was purified by silica gel flash chromatography (0-30% EtOAc in hexanes) to afford 0.065 g (74%) of desired product (2E)-3-[5-ethyl-3-methyl-4-oxo-2-(tetrahydro-2H-pyran-2-yl)-4,5-dihydro-2H-pyrazolo[4,3-c]quinolin-8-yl]acrylaldehyde. LCMS: Rₜ=1.73 min, [MH⁺366.3].

Reductive amination and acidic deprotection as in Example 11 provided the title compound 5-ethyl-3-methyl-8-[(1E)-3-pyrrolidin-1-ylprop-1-en-1-yl]-2,5-dihydro-4H-pyrazolo[4,3-c]quinolin-4-one as light yellow powder. ¹H NMR (400 MHz, CD₃OD) δ 8.25 (d, 1 H), 7.76 (dd, 1 H), 7.58 (d, 1 H), 7.02 (d, 1 H), 6.40-6.51 (m, 1 H), 4.33-4.40 (m, 2 H), 4.04 (d, 2 H), 3.62-3.71 (m, 2 H), 3.16-3.26 (m, 2 H), 2.68 (s, 3 H), 2.01-2.23 (m, 4 H), 1.30-1.35 (m, 3 H). LCMS: Rₜ=0.84 min, [MH⁺337.5].

### Example 23

### Preparation of 8-(3-Hydroxyprop-1-yn-1-yl)-3,5-dimethyl-2,5-dihydro-4H-pyrazolo[4,3-c]quinolin-4-one

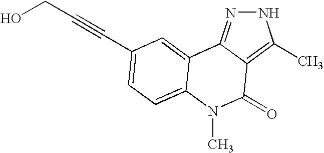

The title compound was prepared using analogous procedures as outlined in Example 1. ¹H NMR (300 MHz, DMSO-d₆) δ 8.17 (s, 1 H), 7.47-7.62 (m, 3 H), 4.32 (s, 2 H), 3.57 (s, 3 H), 2.55 (s, 3 H). LCMS: Rₜ=1.17 min, [MH⁺268.1].

### Example 24

### Preparation of 8-(3-Aminoprop-1-yn-1-yl)-3,5-dimethyl-2,5-dihydro-4H-pyrazolo[4,3-c]quinolin-4-one

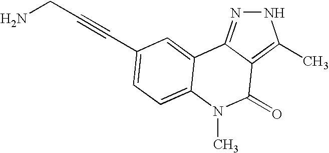

The title compound was prepared using analogous procedures as outlined in Example 1. ¹H NMR (300 MHz, D₂O) δ 7.08 (d, 1 H), 6.94 (s, 1 H), 6.62 (d, 1 H), 4.07 (s, 2 H), 2.93 (s, 3 H), 2.20 (s, 3 H). LCMS: Rₜ=0.86 min, [MH⁺250.1].

### Example 25

### Preparation of 8-Ethynyl-3,5-dimethyl-2,5-dihydro-4H-pyrazolo[4,3-c]quinolin-4-one

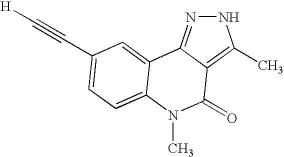

The title compound was prepared using analogous procedures as outlined in Example 1. ¹H NMR (300 MHz, DMSO-d₆) δ 8.21 (s, 1 H), 7.64 (d, 1 H), 7.51 (d, 1 H), 4.23 (s, 1 H), 3.58 (s, 3 H), 2.55 (s, 3 H). LCMS: Rₜ=1.35 min, [MH⁺238.0].

### Example 26

### Preparation of 8-[3-(3,3-Difluoropyrrolidin-1-yl)prop-1-yn-1-yl]-5-ethyl-3-methyl-2,5-dihydro-4H-pyrazolo[4,3-c]quinolin-4-one

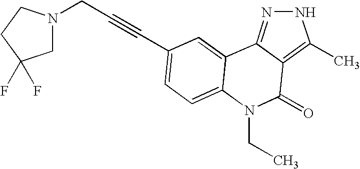

The title compound was prepared using analogous procedures as outlined in Example 11. ¹H NMR (400 MHz, CD₃OD) δ 8.20 (s, 1 H), 7.68 (d, 1 H), 7.52 (d, 1 H), 4.52 (s, 2 H), 4.29 (q, 2 H), 4.05 (app t, 2 H), 3.88 (app t, 2 H), 2.80-2.62 (m, 2 H), 2.61 (s, 3 H), 1.28 (t, 3 H). LCMS: Rₜ=1.25 min, [MH⁺372.2].

### Example 27

### Preparation of 8-(3-Azetidin-1-ylprop-1-yn-1-yl)-5-ethyl-3-methyl-2,5-dihydro-4H-pyrazolo[4,3-c]quinolin-4-one

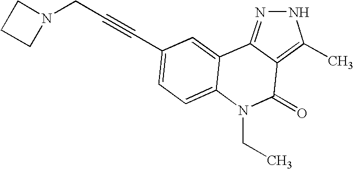

The title compound was prepared using analogous procedures as outlined in Example 11. ¹H NMR (400 MHz, CD₃OD) δ 8.26 (s, 1 H), 7.70 (d, 1 H), 7.58 (d, 1 H), 4.40-4.25 (m, 8 H), 2.69 (s, 3 H), 2.68-2.48 (m, 2 H), 1.33 (t, 3 H). LCMS: Rₜ=0.91 min, [MH⁺321.4].

### Example 28

### Preparation of 8-[3-(Diisopropylamino)prop-1-yn-1-yl]-5-ethyl-3-methyl-2,5-dihydro-4H-pyrazolo[4,3-c]quinolin-4-one

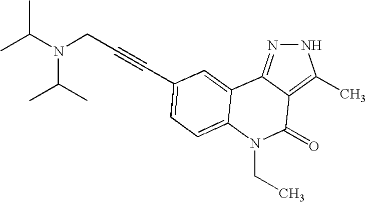

The title compound was prepared using analogous procedures as outlined in Example 11. ¹H NMR (400 MHz, CD₃OD) δ 8.30 (d, 1 H), 7.70 (dd, 1 H), 7.61 (d, 1 H), 4.45 (s, 2 H), 4.40 (q, 2 H), 3.99 (m, 2 H), 2.69 (s, 3 H), 1.52 (t, 12 H), 1.35 (t, 3 H). LCMS: Rₜ=1.06 min, [MH⁺365.3].

### Example 29

### Preparation of 8-{3-[(2R,6S)-2,6-Dimethylpiperidin-1-yl]prop-1-yn-1-yl}-5-ethyl-3-methyl-2,5-dihydro-4H-pyrazolo[4,3-c]quinolin-4-one

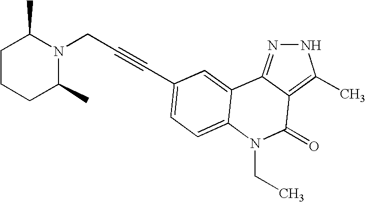

The title compound was prepared using analogous procedures as outlined in Example 11. ¹H NMR (400 MHz, CD₃OD) δ 8.28 (s, 1 H), 7.69 (d, 1 H), 7.60 (d, 1 H), 4.60 (q, 2 H), 4.53 (s, 2 H), 2.90 (s, 3 H), 2.17-2.13 (m, 2 H), 2.07-1.99 (m, 1 H), 1.90-1.71 (m, 3 H), 1.65-1.50 (m, 9 H). LCMS: Rₜ=1.15 min, [MH⁺377.2].

### Example 30

### Preparation of 8-{3-[tert-Butyl(isopropyl)amino]prop-1-yn-1-yl}-5-ethyl-3-methyl-2,5-dihydro-4H-pyrazolo[4,3-c]quinolin-4-one

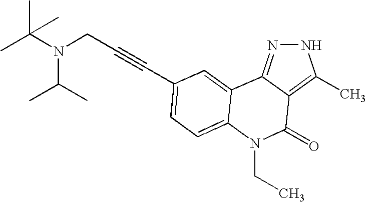

The title compound was prepared using analogous procedures as outlined in Example 11. ¹H NMR (400 MHz, CD₃OD) δ 8.12 (s, 1 H), 7.55 (s, 2 H), 4.37 (q, 2 H), 3.72 (s, 2 H), 3.48 (m, 1 H), 2.67 (s, 3 H), 1.15-1.08 (m, 12 H), 1.03 (d, 6 H). LCMS: Rₜ=1.11 min, [MH⁺379.5].

### Example 31

### Preparation of 8-[3-(tert-Butylamino)-3-methylbut-1-yn-1-yl]-5-ethyl-3-methyl-2,5-dihydro-4H-pyrazolo[4,3-c]quinolin-4-one

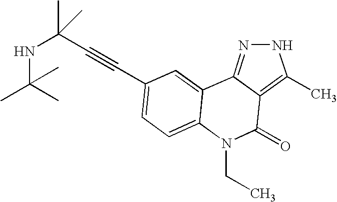

The title compound was prepared using analogous procedures as outlined in Example 1. ¹H NMR (400 MHz, CD₃OD) δ 8.20 (s, 1 H), 7.62-7.60 (m, 2 H), 4.38 (q, 2 H), 2.69 (s, 3 H), 1.75 (s, 6 H), 1.52 (s, 9 H), 1.31 (t, 3 H). LCMS: Rₜ=0.99 min, [MH⁺365.2].

### Example 32

### Preparation of 8-{(1E)-3-[(2S,5S)-2,5-Dimethylpyrrolidin-1-yl]prop-1-en-1-yl}-5-ethyl-3-methyl-2,5-dihydro-4H-pyrazolo[4,3-c]quinolin-4-one (Racemic Mixture of Enantiomers)

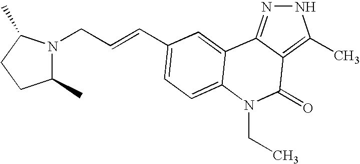

The title compound was prepared using analogous procedures as outlined in Example 22. ¹H NMR (400 MHz, DMSO) δ 10.14 (bs, 1H), 8.26 (s, 1H), 7.71 (d, 1H), 7.58 (d, 1H), 6.99 (d, 1H), 6.40-6.47 (m, 1H), 4.27-4.32. (m, 2H), 3.91-4.04 (m, 2H), 3.76-3.84 (m, 1H), 3.60-3.68 (m, 1H), 2.59 (s, 3H), 2.26-2.35 (m, 1H), 2.10-2.19 (m, 1H), 1.70-1.78 (m, 1H), 1.57-1.66 (m, 1H), 1.41 (d, 3H), 1.30 (d, 3H), 1.22 (t, 3H). LCMS: Method FA, Rₜ=2.80 min, [MH⁻363.2].

### Example 33

### Preparation of 8-{(1E)-3-[(2R,5S)-2,5-Dimethylpyrrolidin-1-yl]prop-1-en-1-yl}-5-ethyl-3-methyl-2,5-dihydro-4H-pyrazolo[4,3-c]quinolin-4-one

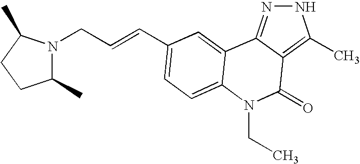

The title compound was prepared using analogous procedures as outlined in Example 22. ¹H NMR (400 MHz, DMSO) δ 9.46 (bs, 1H), 8.23 (s, 1H), 7.72-7.77 (m, 1H), 7.56-7.61 (m, 1H), 7.30 (d, 1H), 6.42-6.49 (m, 1H), 4.27-4.32 (m, 2H), 4.00-4.05 (m, 2H), 3.53-3.61 (m, 2H), 2.59 (s, 3H), 2.14-2.20 (m, 2H), 1.62-1.69 (m, 2H), 1.41-1.42 (m, 6H), 1.21 (t, 3H). LCMS: Rₜ=2.80 min, [MH⁻363.2].

### Example 34

### Preparation of 5-Ethyl-3-methyl-7-(3-pyrrolidin-1-ylpropyl)-2,5-dihydro-4H-pyrazolo[4,3-c]quinolin-4-one

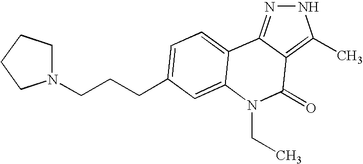

The title compound was prepared using analogous procedures as outlined in Example 1. ¹H NMR (300 MHz, CD₃OD) δ 8.04 (d, 1H), 7.48 (bs, 1H), 7.24-7.27 (m, 1H), 4.36-4.43 (m, 2H), 3.62-3.69 (m, 2H), 3.21-3.26 (m, 2H), 3.02-3.11 (m, 2H), 2.87-2.92 (m, 2H), 2.67 (s, 3H), 1.96-2.19 (m, 6H), 1.30-1.35 (m, 3H). LCMS: Rₜ=0.89 min, [MH⁺339.4].

### Example 35

### Preparation of 8-[(1E)-3-(Diethylamino)prop-1-en-1-yl]-5-ethyl-3-methyl-2,5-dihydro-4H-pyrazolo[4,3-c]quinolin-4-one

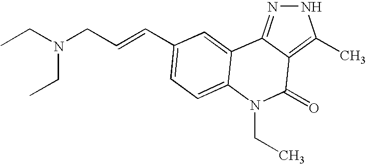

The title compound was prepared using analogous procedures as outlined in Example 22. ¹H NMR (400 MHz, CD₃OD) δ 8.26 (bs, 1H), 7.76 (d, 1H), 7.60 (d, 1H), 7.01 (d, 1H), 6.36-6.43 (m, 1H), 4.37-4.42 (m, 2H), 3.91-3.93 (m, 2H), 3.18-3.24 (s, 3H), 2.69 (s, 3H), 1.32-1.38 (m, 10H). LCMS: Rₜ=0.95 min, [MH⁺ 339.0].

### Example 36

### Preparation of 8-[(1E)-3-(Diisopropylamino)prop-1-en-1-yl]-5-ethyl-3-methyl-2,5-dihydro-4H-pyrazolo[4,3-c]quinolin-4-one

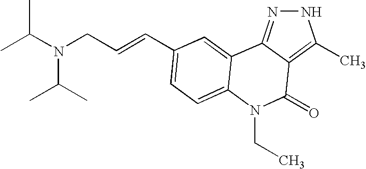

The title compound was prepared using analogous procedures as outlined in Example 22. ¹H NMR (400 MHz, CD₃OD) δ 8.28 (bs, 1H), 7.75-7.73 (m, 1H), 7.60-7.62 (m, 1H), 7.04 (d, 1H), 6.38-6.45 (m, 1H), 4.37-4.42 (m, 2H), 4.07 (d, 2H), 3.83-3.89 (m, 2H), 2.70 (s, 3H), 1.46-1.48 (m, 12H), 1.34 (t, 3H). LCMS: Rₜ=0.98 min, [MH⁻365.6].

### Example 37

### Preparation of 8-{(1E)-3-[Benzyl(methyl)amino]prop-1-en-1-yl}-5-ethyl-3-methyl-2,5-dihydro-4H-pyrazolo[4,3-c]quinolin-4-one

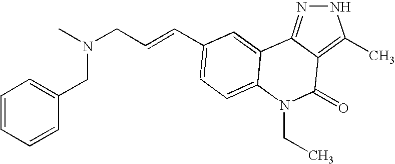

The title compound was prepared using analogous procedures as outlined in Example 22. ¹H NMR (400 MHz, DMSO) δ 11.11 (bs, 1H), 8.35 (s, 1H), 7.73 (dd, 1H), 7.62-7.64 (m, 3H), 7.49-7.51 (m, 3H), 4.23-4.52 (m, 6H), 2.83 (s, 3H), 2.59 (s, 3H), 1.21 (t, 3H). LCMS: Rₜ=1.02 min, [MH⁺385.6].

### Example 38

### Preparation of 5-Ethyl-3-methyl-8-[5-(pyrrolidin-1-ylmethyl)-2-thienyl]-2,5-dihydro-4H-pyrazolo[4,3-c]quinolin-4-one

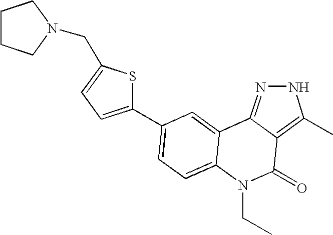

### Step 1, Preparation of 5-[5-Ethyl-3-methyl-4-oxo-2-(tetrahydro-2H-pyran-2-yl)-4,5-dihydro-2H-pyrazolo[4,3-c]quinolin-8-yl]thiophene-2-carbaldehyde

To a solution of 5-ethyl-8-iodo-3-methyl-2-(tetrahydro-2H-pyran-2-yl)-2,5-dihydro-4H-pyrazolo[4,3-c]quinolin-4-one (as prepared in example 1, 1.5 g, 3.43 mmol) in THF (40 mL) and ethanol (10 mL) was added a saturated aq solution of sodium carbonate (1.0 mL). The reaction was then sparged with Ar for 20 min. 5-Formyl-2-thiopheneboronic acid (0.803 g, 5.15 mmol) and tetrakis(triphenylphosphine) palladium (0.198 g, 0.17 mmol) were added to the solution and the reaction mixture was heated to 85° C. and stirred for 5 h. The mixture was then cooled to rt and the residue was diluted with EtOAc and washed with a saturated aq NaHCO₃ solution (50 mL) and water (2×50 mL). The organic layer was dried over MgSO₄, filtered and the solvent evaporated in vacuo. The crude material was purified by silica gel chromatography (0-40% EtOAc in hexanes) to afford 0.833 g of 5-[5-ethyl-3-methyl-4-oxo-2-(tetrahydro-2H-pyran-2-yl)-4,5-dihydro-2H-pyrazolo[4,3-c]quinolin-8-yl]thiophene-2-carbaldehyde in 58% yield. LCMS: Rₜ=1.99 min, [MH⁺ 422.3].

### Step 2, Preparation of 5-Ethyl-3-methyl-8-[5-(pyrrolidin-1-ylmethyl)-2-thienyl]-2-(tetrahydro-2H-pyran-2-yl)-2,5-dihydro-4H-pyrazolo[4,3-c]quinolin-4-one

To a solution of 5-[5-ethyl-3-methyl-4-oxo-2-(tetrahydro-2H-pyran-2-yl)-4,5-dihydro-2H-pyrazolo[4,3-c]quinolin-8-yl]thiophene-2-carbaldehyde (410 mg, 0.974 mmol) in DCE (10 mL) was added sodium triacetoxyborohydride (310 mg, 1.461 mmol) and pyrrolidine (0.081 mL, 0.974 mmol). The reaction was stirred at 60° C. for 2 h and cooled to rt. The reaction was diluted with EtOAc, washed with sodium bicarbonate (saturated aq solution) and brine, dried over MgSO₄, filtered, and concentrated. The crude residue was purified by flash chromatography (gradient elution: 0-10% MeOH in DCM) to afford 325 mg of 5-ethyl-3-methyl-8-[5-(pyrrolidin-1-ylmethyl)-2-thienyl]-2-(tetrahydro-2H-pyran-2-yl)-2,5-dihydro-4H-pyrazolo[4,3-c]quinolin-4-one.

### Step 3, Preparation of 5-Ethyl-3-methyl-8-[5-(pyrrolidin-1-ylmethyl)-2-thienyl]-2,5-dihydro-4H-pyrazolo[4,3-c]quinolin-4-one

To a solution of 5-ethyl-3-methyl-8-[5-(pyrrolidin-1-ylmethyl)-2-thienyl]-2-(tetrahydro-2H-pyran-2-yl)-2,5-dihydro-4H-pyrazolo[4,3-c]quinolin-4-one (50 mg, 0.116 mmol) in MeOH (5 mL) was added 200 μL of concentrated aq HCl. The reaction was stirred for 2 h and concentrated in vacuo. Purification via flash chromatography afforded 44 mg of the title compound. ¹H NMR (400 MHz, CD₃OD) δ 8.40 (d, 1 H), 7.88 (dd, 1 H), 7.61 (d, 1 H), 7.49 (d, 1 H), 7.38 (d, 1 H), 4.68 (s, 2 H), 4.38 (q, 2 H), 3.95-3.60 (m, 4 H), 2.69 (s, 3 H), 2.25-2.02 (m 4 H), 1.35 (t, 3 H). LCMS: Rₜ=0.95 min, [MH⁺393.1].

### Example 39

### Preparation of 5-Ethyl-3-methyl-8-[5-(pyrrolidin-1-ylmethyl)-2-furyl]-2,5-dihydro-4H-pyrazolo[4,3-c]quinolin-4-one

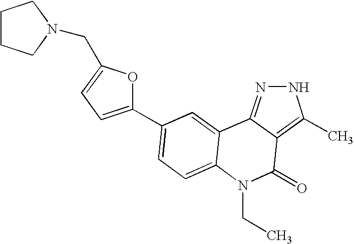

The title compound was prepared using analogous procedures as outlined in Example 38. ¹H NMR (400 MHz, CD₃OD) δ 8.50 (s, 1 H), 7.99 (d, 1 H), 7.62 (d, 2 H), 6.95 (d, 1 H), 6.82 (d, 1 H), 4.59 (s, 2 H), 4.38 (q, 2 H), 3.75-3.62 (m, 2 H), 3.40-3.28 (m, 2 H), 2.70 (s, 3 H), 2.30-2.05 (m, 4 H), 1.35 (t, 3 H). LCMS: Rₜ=1.46 min, [MH⁺377.5].

### Example 40

### Preparation of 5-Ethyl-3-methyl-8-(3-pyrrolidin-1-ylpropyl)-2,5-dihydro-4H-pyrazolo[4,3-c]quinolin-4-one

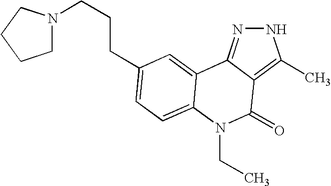

To a solution of 5-ethyl-3-methyl-8-(3-pyrrolidin-1-ylprop-1-yn-1-yl)-2,5-dihydro-4H-pyrazolo[4,3-c]quinolin-4-one (as prepared in example 1, 50 mg, 0.149 mmol) in MeOH (50 mL) was added 10% Pd on carbon (20 mg). The suspension was degassed and backfilled with hydrogen (3×) and stirred under ambient pressure for 1 h. The reaction was then filtered through a pad of Celite and concentrated. The crude residue was then purified via flash chromatography (gradient elution: 0-10% MeOH in DCM) to afford 48 mg of 5-ethyl-3-methyl-8-(3-pyrrolidin-1-ylpropyl)-2,5-dihydro-4H-pyrazolo[4,3-c]quinolin-4-one as a white solid. ¹H NMR (400 MHz, CD₃OD) δ 8.05 (s, 1 H), 7.52-7.50 (m, 2 H), 4.39 (q, 2 H), 3.25 (m, 4 H), 2.85 (t, 2 H), 2.65 (s, 3 H), 2.20-2.05 (m, 6 H), 1.35-1.28 (m, 5 H). LCMS: Rₜ=0.88 min, [MH⁺339.3].

### Example 41

### Preparation of 5-Ethyl-3-methyl-8-(1H-pyrazol-4-yl)-2,5-dihydro-4H-pyrazolo[4,3-c]quinolin-4-one

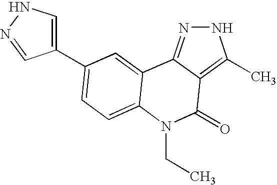

The title compound was prepared using analogous procedures as outlined in Example 38. ¹H NMR (400 MHz, DMSO-d₆) δ 8.37 (s, 1 H), 8.09 (s, 2 H), 7.81 (d, 1 H), 7.56 (d, 1 H), 4.30 (q, 2 H), 2.58 (s, 3 H), 1.24 (t, 3 H). LCMS: Rₜ=1.13 min, [MH⁺294.2].

### Example 42

### Preparation of 5-Ethyl-3-methyl-8-(1-methyl-1H-pyrazol-4-yl)-2,5-dihydro-4H-pyrazolo[4,3-c]quinolin-4-one

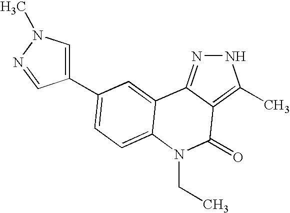

The title compound was prepared using analogous procedures as outlined in Example 38. ¹H NMR (400 MHz, CD₃OD) δ 8.29 (d, 2 H), 8.20 (s, 1 H), 7.82 (d, 1 H), 7.60 (d, 1 H), 4.38 (q, 2 H), 4.08 (s, 3 H), 2.70 (s, 3 H), 1.34 (t, 3 H). LCMS: Rₜ=1.21 min, [MH⁺308.2].

### Example 43

### Preparation of 8-{5-[(Dimethylamino)methyl]-2-thienyl}-5-ethyl-3-methyl-2,5-dihydro-4H-pyrazolo[4,3-c]quinolin-4-one

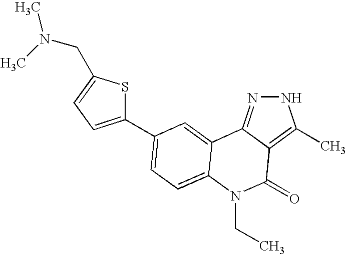

The title compound was prepared using analogous procedures as outlined in Example 38. ¹H NMR (400 MHz, CD₃OD) δ 8.45 (s, 1 H), 7.90 (d, 1 H), 7.64 (d, 1 H), 7.51 (d, 1 H), 7.38 (d, 1 H), 4.62 (s, 2 H), 4.40 (q, 2 H), 2.95 (s, 6 H), 2.70 (s, 3 H), 1.37 (t, 3 H). HRMS: [MH⁺367.1605].

### Example 44

### Preparation of 5-(2-Fluoroethyl)-3-methyl-8-[5-(pyrrolidin-1-ylmethyl)-2-thienyl]-2,5-dihydro-4H-pyrazolo[4,3-c]quinolin-4-one

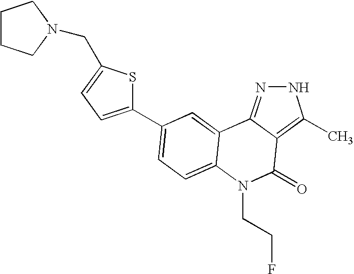

The title compound was prepared using analogous procedures as outlined in Example 38. ¹H NMR (400 MHz, CD₃OD) δ 8.43 (s, 1 H), 7.85 (dd, 1 H), 7.70 (dd, 1 H), 7.49 (d, 1 H), 7.39 (d, 1 H), 4.88-4.62 (m, 6 H), 3.65-3.22 (m, 4 H), 2.68 (s, 3 H), 2.28-2.01 (m, 4 H). LCMS: Rₜ=0.99 min, [MH⁺411.2].

### Example 45

### Preparation of 5-Ethyl-3-methyl-4-methylene-8-[5-(piperidin-1-ylmethyl)-2-thienyl]-4,5-dihydro-2H-pyrazolo[4,3-c]quinoline

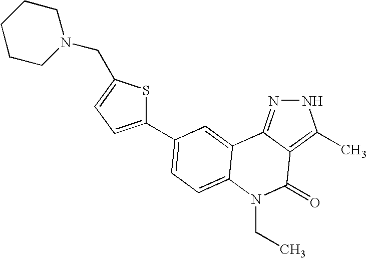

The title compound was prepared using analogous procedures as outlined in Example 38. ¹H NMR (400 MHz, DMSO-d₆) δ 8.43 (br s, 1 H), 7.86 (dd, 1 H), 7.64 (d, 1 H), 7.57 (d, 1 H), 7.40 (d, 1 H), 4.54 (d, 2 H), 4.28-4.34 (m, 2 H), 3.41 (d, 2 H), 2.86-2.95 (m, 2 H), 2.59 (s, 3 H), 1.78-1.87 (m, 3 H), 1.66-1.78 (m, 3 H), 1.23 (t, 3 H). LCMS: Rₜ=1.05 min, [MH⁺322.4].

### Example 46

### Preparation of 8-[5-(3,4-Dihydroisoquinolin-2(1H)-ylmethyl)-2-thienyl]-5-ethyl-3-methyl-2,5-dihydro-4H-pyrazolo[4,3-c]quinolin-4-one

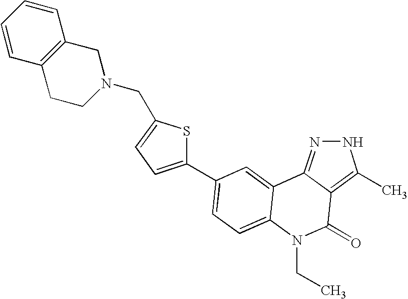

The title compound was prepared using analogous procedures as outlined in Example 38. ¹H NMR (400 MHz, CD₃OD) δ 8.47 (d, 1 H), 7.91 (dd, 1 H), 7.66 (d, 1 H), 7.54 (d, 1 H), 7.43 (d, 1 H), 7.27-7.36 (m, 3 H), 7.23 (d, 1 H), 4.79 (d, 2 H), 4.52 (d, 2 H), 4.45-4.38 (m, 2 H), 3.48 (m, 2 H), 3.24 (m, 2 H), 2.70 (s, 3 H), 1.36 (t, 3 H). LCMS: Rₜ=1.15 min, [MH⁺355.0].

### Example 47

### Preparation of 8-{5-[(Diethylamino)methyl]-2-thienyl}-5-ethyl-3-methyl-2,5-dihydro-4H-pyrazolo[4,3-c]quinolin-4-one

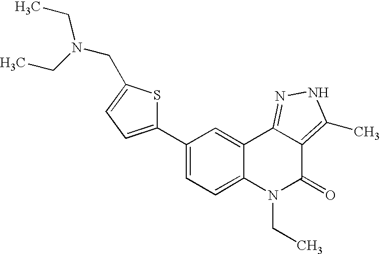

The title compound was prepared using analogous procedures as outlined in Example 38. ¹H NMR (300 MHz, CD₃OD) δ 8.43 (d, 1 H), 7.89 (dd, 1 H), 7.63 (d, 1 H), 7.50 (d, 1 H), 7.40 (d, 1 H), 4.65 (s, 2 H), 4.36-4.43 (m, 2 H), 3.24-3.65 (m, 4 H), 2.69 (s, 3 H), 1.40-1.44 (m, 6 H), 1.29-1.37 (m, 3 H). LCMS: Rₜ=0.98 min, [MH⁺395.0].

### Example 48

### Preparation of 8-{5-[(Dimethylamino)methyl]-2-thienyl}-3-methyl-5-propyl-2,5-dihydro-4H-pyrazolo[4,3-c]quinolin-4-one

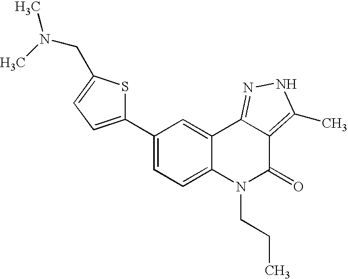

The title compound was prepared using analogous procedures as outlined in Example 38. ¹H NMR (400 MHz, CD₃OD) δ 8.42 (s, 1 H), 7.89 (dd, 1 H), 7.60 (d, 1 H), 7.51 (d, 1 H), 7.38 (d, 1 H), 4.62 (s, 2 H), 4.29 (app t, 2 H), 2.95 (s, 6 H), 2.69 (s, 3 H), 1.85-1.70 (m, 2 H), 1.05 (t, 3 H). LCMS: Rₜ=1.054 min, [MH⁺381.1].

### Example 49

### Preparation of 3-Methyl-8-[5-(piperidin-1-ylmethyl)-2-thienyl]-5-propyl-2,5-dihydro-4H-pyrazolo[4,3-c]quinolin-4-one

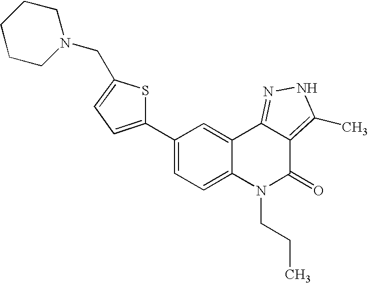

The title compound was prepared using analogous procedures as outlined in Example 38. ¹H NMR (400 MHz, CD₃OD) δ 8.40 (s, 1 H), 7.88 (dd, 1 H), 7.60 (d, 1 H), 7.50 (d, 1 H), 7.37 (d, 1 H), 4.59 (s, 2 H), 4.28 (app t, 2 H), 3.59 (d, 2 H), 3.05 (app t, 2 H), 2.70 (s, 3 H), 2.08-1.95 (m, 2 H), 1.91-1.50 (m, 6 H), 1.05 (t, 3 H). LCMS: Rₜ=1.123 min, [MH⁺421.2].

### Example 50

### Preparation of 8-[5-(Azetidin-1-ylmethyl)-2-thienyl]-3-methyl-5-propyl-2,5-dihydro-4H-pyrazolo[4,3-c]quinolin-4-one

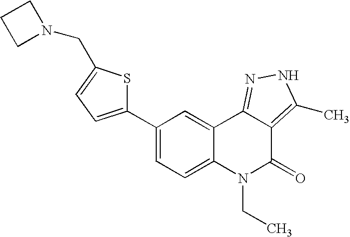

The title compound was prepared using analogous procedures as outlined in Example 38. ¹H NMR (400 MHz, CD₃OD) δ 8.29 (s, 1 H), 7.77 (d, 1 H), 7.52 (d, 1 H), 7.29 (d, 1 H), 6.99 (d, 1 H), 4.35 (q, 2 H), 3.85 (s, 2 H), 3.41 (t, 4 H), 2.65 (s, 3 H), 2.20-2.10 (m, 2 H), 1.32 (t, 3 H). LCMS: Rₜ=1.031 min, [MH⁺379.4].

### Example 51

### Preparation of 8-{5-[(3,3-Difluoropyrrolidin-1-yl)methyl]-2-thienyl}-5-ethyl-3-methyl-2,5-dihydro-4H-pyrazolo[4,3-c]quinolin-4-one

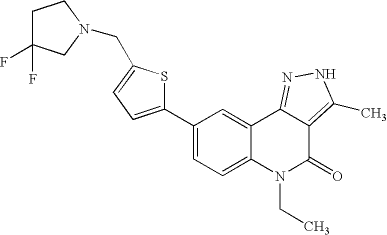

The title compound was prepared using analogous procedures as outlined in Example 38. ¹H NMR (400 MHz, CD₃OD) δ 8.45-8.35 (m, 1 H), 7.92-7.80 (m, 1 H), 7.66-7.39 (m, 3 H), 4.90-4.71 (m, 2 H), 4.41-4.29 (m, 2 H), 4.10-3.75 (m, 3 H), 2.80-2.61 (m, 6 H), 1.40-1.29 (m, 3 H). LCMS: Rₜ=1.28 min, [MH⁺429.4].

### Example 52

### Preparation of 5-Ethyl-8-{5-[(3-hydroxyazetidin-1-yl)methyl]-2-thienyl}-3-methyl-2,5-dihydro-4H-pyrazolo[4,3-c]quinolin-4-one

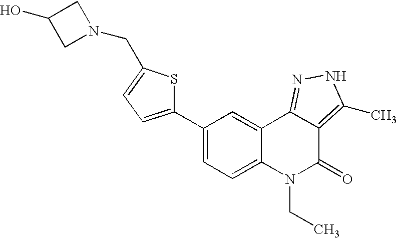

The title compound was prepared using analogous procedures as outlined in Example 38. ¹H NMR (400 MHz, CD₃OD) δ 8.45 (s, 1 H), 7.90 (d, 1 H), 7.64 (d, 1 H), 7.47 (d, 1 H), 7.35 (d, 1 H), 4.77-4.62 (m, 3 H), 4.49-4.37 (m, 4 H), 4.09-4.00 (m, 2 H), 2.72 (s, 3 H), 1.37 (t, 3 H). LCMS: Rₜ=0.92 min, [MH⁺322.3].

### Example 53

### Preparation of 8-(5-{[(2S,5S)-2,5-dimethylpyrrolidin-1-yl]methyl}-2-thienyl)-5-ethyl-3-methyl-2,5-dihydro-4H-pyrazolo[4,3-c]quinolin-4-one (Racemic Mixture of Entiomers)

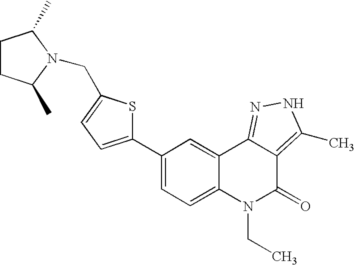

The title compound was prepared using analogous procedures as outlined in Example 38. ¹H NMR (400 MHz, CD₃OD) δ 8.34 (br s, 1 H), 7.85 (d, 1 H), 7.59 (d, 1 H), 7.41 (d, 1 H), 7.22 (d, 1 H), 4.35-4.40 (m, 4 H), 3.69 (br s, 1 H), 3.22 (br s, 1 H), 2.68 (s, 3 H), 2.18 (d, 2 H), 1.65 (d, 2 H), 1.31-1.36 (m, 9 H). LCMS: Rₜ=1.05 min, [MH⁺421.0].

### Example 54

### Preparation of 8-[5-(Aminomethyl)-2-thienyl]-5-ethyl-3-methyl-2,5-dihydro-4H-pyrazolo[4,3-c]quinolin-4-one

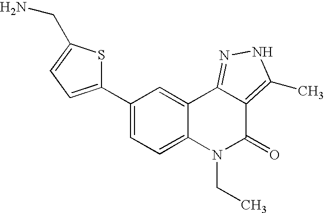

The title compound was prepared using analogous procedures as outlined in Example 38. ¹H NMR (400 MHz, CD₃OD) δ 8.44 (d, 1H), 7.88 (dd, 1H), 7.64 (d, 1H), 7.44-7.45 (m, 1H), 7.26-7.27 (m, 1H), 4.38-4.44 (m, 4H), 2.70 (s, 3H), 1.34-1.38 (m, 3H). LCMS: Rₜ=0.95 min, [MH⁺322.4].

### Example 55

### Preparation of 8-{5-[2-(Diethylamino)ethyl]-2-thienyl}-5-ethyl-3-methyl-2,5-dihydro-4H-pyrazolo[4,3-c]quinolin-4-one

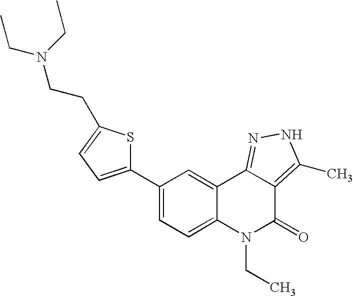

The title compound was prepared using analogous procedures as outlined in Example 38. ¹H NMR (400 MHz, DMSO) δ 8.38-8.40 (m, 1H), 7.80-7.82 (m, 1H), 7.59-7.61 (m, 1H), 7.43-7.44 (m, 1H), 7.07-7.08 (m, 1H), 4.27-4.32 (m, 3H), 3.28-3.38 (m, 4H), 3.15-3.24 (m, 4H), 2.59 (s, 3H), 1.21-1.28 (m, 9H). LCMS: Rₜ=1.01 min, [MH⁺409.0].

### Example 56

### Preparation of 5-Ethyl-8-(5-{[(3S)-3-hydroxypyrrolidin-1-yl]methyl}-2-thienyl)-3-methyl-2,5-dihydro-4H-pyrazolo[4,3-c]quinolin-4-one

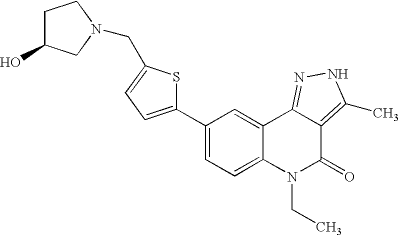

The title compound was prepared using analogous procedures as outlined in Example 38. ¹H NMR (400 MHz, CD₃OD) δ 8.15-8.16 (m, 1H), 7.65 (dd, 1H), 7.38 (d, 1H), 7.19 (d, 1H), 6.93 (d, 1H), 4.37-4.42 (m, 1H), 4.22-4.28 (s, 2H), 3.79-3.88 (m, 2H), 2.80-2.92 (m, 2H), 2.58-2.67 (m, 5H), 2.13-2.22 (m, 1H), 1.73-1.80 (m, 1H), 1.29 (t, 3H). LCMS: Rₜ=0.97 min, [MH⁺409.4].

### Example 57

### Biological Assays

Chk1 Expression & Purification:

Recombinant human Chk1 was expressed as a fusion protein with glutathione S-transferase at the amino-terminus (GST-Chk1) using standard baculovirus vectors and (Bac-to-Bac®) insect cell expression system purchased from GIBCO™ Invitrogen. Recombinant protein expressed in insect cells was purified using glutathione sepharose (Amersham Biotech) using standard procedures described by the manufacturer.

Chk1 FlashPlate® Kinase Assay:

Assays (25 μL) contained 8.7 nM GST-Chk1, 10 mM MES, 0.1 mM ethylene glycol-bis(β-aminoethylether)-N,N,N′,N′-tetracetic acid (EGTA, pH 8.0), 2 mM DTT, 0.05% Tween 20, 3 μM peptide substrate (Biotin-ILSRRPSYRKILND-free acid) (SEQ ID NO: 1), 1 μM ATP, 0.4 uCi ³³P-γ-ATP (NEN), 4% DMSO. Reactions were incubated for 30 minutes at room temperature, terminated with 50 μL of 50 mM EDTA and 90 μL were transferred to streptavidin-coated FlashPlates® (NEN) and incubated for 1 hour at room temperature. Plates were washed with phosphate buffered saline containing 0.01% Tween-20 and 10 mM sodium pyrophosphate. Plates were dried, sealed with Topseal™ (NEN) and amount of ³³P incorporated into the peptide substate measure using a Packard Topcount® NXT™ scintillation counter with standard settings.

Chk1 DELFIA® Kinase Assay:

Assays (25 μL) utilized 6.4 nM GST-Chk1 containing 25 mM Tris, pH 8.5, 20% glycerol, 50 mM sodium chloride (NaCl), 0.1% Surfact-Amps® 20, 1 μM peptide stubstrate (Biotin-GLYRSPSMPEN-amide) (SEQ ID NO: 2), 2 mM DTT, 4% DMSO, 12.5 μM ATP, 5 mM MgCl₂ and reacted for 30 minutes at room temperature. Reactions were terminated with 100 μL of Stop buffer containing 1% BSA, 10 mM Tris, pH 8.0, 150 mM NaCl, 100 mM EDTA. Stopped reactions (100 μL) were transferred to 96 well neutravidin plates (Pierce) to capture the biotin-peptide substrate during a 30 minute room temperature incubation. Wells were washed and reacted with 100 μL PerkinElmer Wallac Assay Buffer containing 21.5 ng/ml anti-phospho-Ser216-Cdc25c rabbit polyclonal antibody from Cell Signaling Technology (Beverly, Mass.) and 292 ng/ml europium labeled anti-rabbit-IgG for 1 hour at room temperature. Wells were washed and europium released from the bound antibody by addition of Enhancement Solution (100 μL) (PerkinElmer Wallac) and detected using a Wallac Victor2™ using standard manufacturer settings.

Chk1 DELFIA® Kinase Assay:

Assays (25 μL) utilized 2 nM GST-Chk1 containing 10 mM Tris, pH 7.5, 20% glycerol, 50 mM sodium chloride (NaCl), 0.01% Surfact-Amps® 20, 1 μM peptide stubstrate (Biotin-GLYRSPSMPEN-amide) (SEQ ID NO: 2), 01.% BSA, 2 mM DTT, 4% DMSO, 600 μM ATP, 10 mM MgCl₂ and reacted for 50 minutes at room temperature. Reactions were terminated with 100 μL of Stop buffer containing 1% BSA, 10 mM Tris, pH 8.0, 150 mM NaCl, 100 mM EDTA. Stopped reactions (100 μL) were transferred to 96 well NeutrAvidin plates (Pierce) to capture the biotin-peptide substrate during a 30 minute room temperature incubation. Wells were washed and reacted with 100 μL PerkinElmer Wallac Assay Buffer containing 21.5 ng/ml anti-phospho-Ser216-Cdc25c rabbit polyclonal antibody from Cell Signaling Technology (Beverly, Mass.) and 292 ng/ml europium labeled anti-rabbit-IgG for 1 hour at room temperature. Wells were washed and europium released from the bound antibody by addition of Enhancement Solution (100 μL) (PerkinElmer Wallac) and detected using a Perkin Elmer Wallac Envison™ 2100 multilabel reader using standard manufacturer settings.

Compounds I-1 to I-25, I-30, I-31, I-36 to I-41, I-43 to I-47, I-49, I-50, I-53, 1-55, I-56, 1-74 to I-78, I-82 and I-83 were tested in this assay and exhibited IC₅₀ values less than 500 nM. Compounds I-42, I-48, I-51, I-52 and I-54 exhibited IC₅₀ values greater than 500 nM and less than 1 μM.

While this invention has been particularly shown and described with references to preferred embodiments thereof, it will be understood by those skilled in the art that various changes in form and details may be made therein without departing from the scope of the invention encompassed by the appended claims.

What is claimed is:

1. A compound represented by the following structural formula: 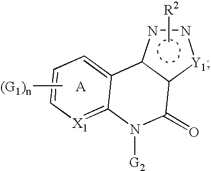
Ring A is optionally substituted at any one or more substitutable ring carbon atoms;
Y₁ is N or CR³;
G₂ is —H, or a C1-C8 aliphatic group optionally substituted with one or more fluoro, —OR¹², —CONR¹¹R¹², —COOR¹², cycloalkyl or phenyl, wherein the cycloalkyl and phenyl are optionally substituted with halo or alkyl;
R² is —H or a group that is cleavable in vivo;
R³ is —H, halogen, alkyl, haloalkyl or -V₁-R⁷, wherein V₁ is a covalent bond or a C1-C4 alkylidene optionally substituted with one or more —OR¹⁴, —NR¹⁵R¹⁶, alkyl, hydroxyalkyl, alkoxyalkyl, aminoalkyl, or with a spiro cycloalkyl group; R⁷ is —OR¹⁴, —SR¹⁴, —CONR¹⁵R¹⁶, —NR¹⁵R¹⁶, —NHC(O)NR¹⁵R¹⁶, —CN, —COOR¹⁴, —NHC(O)H, —NHC(O)R¹⁴, —OC(O)R¹⁴, —C(O)NR¹⁵R¹⁶, —NHC(O)—OR¹⁴, —S(O)₂NR¹⁵R¹⁶, —S(O)₂(R¹⁴), boronate, alkyl boronate, —C(═NR¹⁴)—NR¹⁵R¹⁶, —NH—C(═NR¹⁴)NR¹⁵R¹⁶, —NH—C(═NR¹⁴)R¹⁴, an optionally substituted cycloaliphatic or non-aromatic heterocyclic group, or an optionally substituted aromatic group; R¹⁴ is —H, alkyl or an optionally substituted aromatic or aralkyl group; and R¹⁵ and R¹⁶ are independently —H, alkyl or an optionally substituted aromatic or aralkyl group; or —NR¹⁵R¹⁶ is an optionally substituted nitrogen-containing non-aromatic heterocyclic group;
X₁ is N, or CR⁴;
R⁴ is —H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl);
each G₁ is independently —R^{13b}, -V₃-R¹³, -V₃-R^{13a}, -T₀-T₁-V₃-R¹³, -T₀-T₁-V₃-R^{13a}, -T₀-T₁-R^{13a}, -T₀-Cy-V₄-R¹³, -T₀-Cy-V₄-R^{13a}, -T₀-Cy-T₁-V₄-R¹³, -T₀-Cy-T₁-V₄-R^{13a}, -T₀-Cy-R¹³, or -T₀-Cy-R^{13a}; or n is 2, one G₁ is (-T₂-R²⁰⁰)ₓ and the other G₁ is (-T₃-V₅-R⁵⁰)_{y}, x is 1 or 2, y is 0 or 1 and x+y is 1 or 2; and
T₀ is absent, —CH₂—, —CH₂—CH₂—, or —CH₂—CH₂—CH₂—;
T₁ is —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂;
T₂ is a covalent bond, —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂—;
T₃ is a covalent bond, —O—, —NH—, —C(O)O—, —C(O)— or —C(O)NH—;
Cy is an optionally substituted arylene group or an optionally substituted non-aromatic heterocyclene or non-aromatic carbocyclene group;
V₃ is an optionally substituted C1-C8 alkylidene, provided that V₃ is a C2-C8 alkylidene when T₁ is —O—, —N(R⁶)—, —C(O)O—, or —C(O)N(R⁶)— and R¹³ is —CN, —OR¹², —NR¹¹R¹², —NR¹¹C(O)R¹², —OC(O)R¹², —NR¹¹C(O)NR¹¹R¹², —OC(O)NR¹¹R¹² or —NR¹¹C(O)OR¹², and wherein V₃ is optionally substituted with alkyl, halo, haloalkyl, alkoxy, hydroxy, NR¹¹R¹² or oxo;
V₄ is an optionally substituted bivalent C1-C8 aliphatic group provided that V₄ is a C2-C8 aliphatic group when T₁ is —O—, —N(R⁶)—, —C(O)O—, or —C(O)N(R⁶)— and R¹³ is —CN, —OR¹², —NR¹¹R¹², —NR¹¹C(O)R¹², —OC(O)R¹², —NR¹¹C(O)NR¹¹R¹², —OC(O)NR¹¹R¹² or —NR¹¹C(O)OR¹², and wherein V₄ is optionally substituted with alkyl, halo, haloalkyl, alkoxy, hydroxy, NR¹¹R¹² or oxo;
V₅ is a covalent bond or a C1-C4 alkylidene, provided that V₅ is C2-C4 alkylidene when T₃ is —O—, —NH—, —C(O)O—, or —C(O)NH— and R⁵⁰ is —CN, —OH, —NR⁵¹R⁵², —NHC(O)R⁵¹, —OC(O)R⁵¹, —NHC(O)NR⁵¹R⁵², —OC(O)NR⁵¹R⁵², —NHC(O)OR⁵¹ or a substituted or unsubstituted nitrogen-containing non-aromatic heterocyclic group wherein a C1-C4 alkylidene group represented by V₅ is optionally substituted with a spirocyclopropyl group or one or two methyl groups and wherein a C1-C4 alkylidene group represented by V₅ is optionally fused to a cyclopropyl group;
each R⁶ is independently —H or C1-C3 alkyl;
each R¹¹ is independently —H or a C1-C3 alkyl group;
each R¹² is independently —H or an optionally substituted alkyl, aromatic, aralkyl, non-aromatic heterocyclic or non-aromatic heterocyclylalkyl group; or —NR¹¹R¹² is an optionally substituted aromatic or non-aromatic nitrogen-containing heterocyclic group;
R¹³ is —OR¹², —CN, —COOR¹², —NR¹¹R¹², —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —O—C(O)—OR¹², —OC(O)—NR¹¹R¹², —NR¹¹CO—CH(OR¹⁸)—R¹², —NR¹¹CO—CH(NR¹⁸R¹⁸)—R¹², —NR¹¹CO—(CH₂)ₘCH(NR¹⁸R¹⁸)—R¹², —OC(O)—CH(OR¹⁸)—R¹², —OC(O)—CH(NR¹⁸R¹⁸)—R¹², —NR¹¹CO—C(R¹⁹R¹⁹)—OR¹², —NR¹¹CO—C(R¹⁹R¹⁹)—NR¹¹R¹², —OC(O)—C(R¹⁹R¹⁹)—OR¹², —OC(O)—C(R¹⁹R¹⁹)—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², —C(O)NR¹¹R¹², —NHC(O)NR¹¹R¹², or —C(═NR¹¹)—NR¹¹R¹²;
R^{13a} is an optionally substituted nitrogen-containing heteroaromatic group or a nitrogen-containing non-aromatic heterocyclic group;
R^{13b} is an optionally substituted nitrogen-containing heteroaromatic group or a nitrogen-containing non-aromatic heterocyclic group;
each R¹⁸ is independently —H, a C1-C3 alkyl group, —C(O)H, —C(O)—(C1-C3 alkyl), —C(O)NH₂, —C(O)NH—(C1-C3 alkyl), —C(O)N—(C1-C3 alkyl)₂, —C(O)O—(C1-C3 alkyl), —S(O)₂(C1-C3 alkyl) or —NR¹⁸R¹⁸ taken together is a substituted or unsubstituted non-aromatic nitrogen-containing heterocyclic group;
each R¹⁹ is independently —H, a C1-C3 alkyl group or —C(R¹⁹R¹⁹)— taken together is a C3-C8 cycloalkyl group;
R⁵⁰ is —CN, —OR⁵¹, —NR⁵¹R⁵², —C(O)NR⁵¹R⁵², —NHC(O)R⁵¹, —NHC(O)NR⁵¹R⁵², —NHC(O)OR⁵¹, —C(O)OR⁵¹ or an optionally substituted aromatic group or non-aromatic heterocyclic group;
each R⁵¹ and each R⁵² are independently —H or C1-C3 alkyl or —NR⁵¹R⁵² is an optionally substituted non-aromatic heterocyclic group;
R²⁰⁰ is an optionally substituted C2-C4 alkenyl or C2-C4 alkynyl group;
m is 1 or 2; and
n is 1 or 2.

2. The compound of claim 1 wherein each G₁ is independently —R^{13b}, -V₃-R¹³, -V₃-R^{13a}, -T₀-T₁-V₃-R¹³, -T₀-T₁-V₃-R^{13a}, -T₀-T₁-R^{13a}, -T₀-Cy-V₄-R¹³, -T₀-Cy-V₄-R^{13a}, -T₀-Cy-T₁-V₄-R¹³, -T₀-Cy-T₁-V₄-R^{13a}, T₀-Cy-R¹³, or -T₀-Cy-R^{13a}.

3. The compound of claim 2, wherein the compound is represented by any of the following structural formulae: 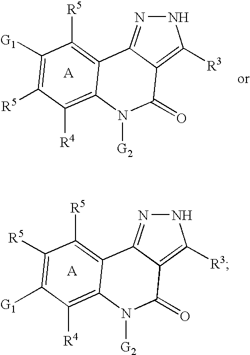
or a pharmaceutically acceptable salt thereof, wherein:
G₂ is C1-C4 alkyl, optionally substituted with fluoro or a C3-C8 cycloalkyl optionally substituted with halo or alkyl; and
each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl).

4. The compound of claim 3, wherein:
R³ is methyl, or ethyl; or R³ is V₁-R⁷, wherein V₁ is a C1-C2 alkylidene and R⁷ is —OH, —OCH₃; or V₁ is a covalent bond and R⁷ is cyclopropyl, cyclopentyl, furyl or tetrahydrofuryl; and
R⁴ and each R⁵ are independently —H, halogen, C1-C3 alkyl, C1-C3 haloalkyl, C1-C3 alkoxy, or C1-C3 haloalkoxy.

5. The compound of claim 3, wherein
G₁ is —R^{13b};
R^{13b} is an optionally substituted imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl or thiadiazolyl;
each substitutable ring nitrogen atom of the group represented by R^{13b} is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group;
each substitutable ring carbon atom of a non-aromatic ring in the group represented by R^{13b} is optionally substituted with a C1-C3 alkyl group, hydroxy, fluoro, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 fluoroalkylamido, amino (C1-C3) alkyl, (C1-C3)alkylamino(C1-C3)alkyl, (C1-C3)dialkylamino(C1-C3)alkyl, hydroxy(C1-C3)alkyl, (C1-C3)alkoxy(C1-C3)alkyl;
each substitutable ring carbon atom of an aromatic ring in the group represented by R^{13b} is optionally substituted with halo, hydroxy, cyano, C1-C3 alkyl, C1-C3 fluoroalkyl, C1-C3 alkoxy, C1-C3 fluoroalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, —NR′C(O)O—(C1-C3 alkyl)-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl); and
each R′ is hydrogen or a C1-C3 alkyl group.

6. The compound of claim 3, wherein G₁ is -V₃-R¹³, -V₃-R^{13a}, -T₀-T₁-V₃-R¹³, -T₀-T₁-V₃-R^{13a}, or -T₀-T₁-R^{13a}.

7. The compound of claim 6, wherein:
V₃ is C1-C4 alkylidene;
R¹³ is —CN, —OR¹², —NR¹¹R¹², —NHC(O)R¹², —NHC(O)OR¹², —NHC(O)NR¹¹R¹², —NHC(O)OR¹², or —OC(O)R¹²;
each substitutable ring nitrogen atom of the group represented by R^{13a} is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group;
each substitutable ring carbon atom of a non-aromatic ring in the group represented by R^{13a} is optionally substituted with a C1-C3 alkyl group, hydroxy, fluoro, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 fluoroalkylamido, amino (C1-C3) alkyl, (C1-C3)alkylamino(C1-C3)alkyl, (C1-C3)dialkylamino(C1-C3)alkyl, hydroxy(C1-C3)alkyl, (C1-C3)alkoxy(C1-C3)alkyl;
each substitutable ring carbon atom of an aromatic ring in the group represented by R^{13a} is optionally substituted with halo, hydroxy, cyano, C1-C3 alkyl, C1-C3 fluoroalkyl, C1-C3 alkoxy, C1-C3 fluoroalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, —NR′C(O)O—(C1-C3 alkyl)-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl); and
each R′ is hydrogen or a C1-C3 alkyl group.

8. The compound of claim 7, wherein:
T₀ is absent;
T₁ is —O— or —N(R⁶);
R³ is —H, methyl, ethyl, n-propyl, iso-propyl, C1-C3 haloalkyl or V₁-R⁷, wherein V₁ is a covalent bond or a C1-C2 alkylidene optionally substituted with one or two methyl groups or with a spiro cyclopropyl group; and R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl; and
R^{13a} is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl, and azabicyclopentyl, azabicyclohexyl, azabicycloheptyl, azabicyclooctyl, azabicyclononyl, azabicyclodecyl, diazabicyclohexyl, diazabicycloheptyl, diazabicyclooctyl, diazabicyclononyl, or diazabicyclodecyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl, and thiadiazolyl.

9. The compound of claim 8, wherein:
R³ is methyl, ethyl; or R³ is V₁-R⁷, wherein V₁ is a C1-C2 alkylidene and R⁷ is —OH, —OCH₃; or V₁ is a covalent bond and R⁷ is cyclopropyl, cyclopentyl, furyl or tetrahydrofuryl;
R⁴ and each R⁵ are independently —H, halogen, C1-C3 alkyl, C1-C3 haloalkyl, C1-C3 alkoxy, or C1-C3 haloalkoxy;
R¹³ is —OH, —CN, C1-C3 alkoxy, NH₂, C1-C3 alkylamino, C1-C3 dialkylamino, C1-C3 hydroxyalkyl, or C1-C3 haloalkylamino; and
R^{13a} is an optionally substituted non-aromatic heterocyclic group selected from N-pyrrolidinyl, N-piperidinyl, N-morpholinyl, N-piperazinyl, N-thiomorpholinyl, N-azetidinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl and 3-oxo-N-8-azabicyclo[3.2.1]octyl or N-8-azabicyclo[3.2.1]octyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl and thiadiazolyl.

10. The compound of claim 3, wherein G₁ is -T₀-Cy-V₄-R¹³, -T₀-Cy-V₄-R^{13a}, -T₀-Cy-T₁-V₄-R¹³, -T₀-Cy-T₁-V₄-R^{13a}, -T₀-Cy-R¹³, or -T₀-Cy-R^{13a}.

11. The compound of claim 10, wherein:
T₀ is absent;
V₄ is C1-C4 alkylidene, alkenylidene or alkynylidene group optionally substituted with C1-C3 alkyl;
R¹³ is —CN, —OR¹², —NR¹¹R¹², —NHC(O)R¹², —NHC(O)OR¹², —NHC(O)NR¹¹R¹², —NHC(O)OR¹², or —OC(O)R¹²;
each substitutable ring nitrogen atom of the group represented by R^{13a} or Cy is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group;
each substitutable ring carbon atom of a non-aromatic ring in the group represented by R^{13a} or Cy is optionally substituted with a C1-C3 alkyl group, hydroxy, fluoro, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 fluoroalkylamido, amino (C1-C3) alkyl, (C1-C3)alkylamino(C1-C3)alkyl, (C1-C3)dialkylamino(C1-C3)alkyl, hydroxy(C1-C3)alkyl, (C1-C3)alkoxy(C1-C3)alkyl;
each substitutable ring carbon atom of an aromatic ring in the group represented by R^{13a} or Cy is optionally substituted with halo, hydroxy, cyano, C1-C3 alkyl, C1-C3 fluoroalkyl, C1-C3 alkoxy, C1-C3 fluoroalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, —NR′C(O)O—(C1-C3 alkyl)-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl); and
each R′ is hydrogen or a C1-C3 alkyl group.

12. The compound of claim 11, wherein:
V₄ is C1-C4 alkylidene;
R³ is —H, methyl, ethyl, n-propyl, iso-propyl, C1-C3 haloalkyl or V₁-R⁷, wherein V₁ is a covalent bond or a C1-C2 alkylidene optionally substituted with one or two methyl groups or with a spiro cyclopropyl group; and R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl;
R^{13a} is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl and azabicyclopentanyl, azabicyclohexanyl, azabicycloheptanyl, azabicyclononanyl, azabicyclodecanyl, diazabicyclohexanyl, diazabicycloheptanyl, diazabicyclooctanyl, diazabicyclononanyl, or diazabicyclodecanyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl and thiadiazolyl; and
Cy is an optionally substituted phenylene, pyrrolylene, thienylene, furanylene, imidazolylene, triazolylene, tetrazolylene oxazolylene, isoxazolylene, oxadiazolylene, pyrazolylene, pyridinylene, pyrimidylene, pyrazinylene, thiazolylene, cyclopropylene, cyclopentylene, cyclohexylene, cycloheptylene, piperidinylene, piperazinylene, pyrrolidinylene, pyrazolidinylene, imidazolidinylene, tetrahydrofuranylene, tetrahydrothienylene, isooxazolidinylene, oxazolidinylene, isothiazolidinylene, thiazolidinylene, oxathiolanylene, dioxolanylene, or dithiolanylene.

13. The compound of claim 12, wherein:
R³ is methyl or ethyl; or R³ is V₁-R⁷, wherein V₁ is a C1-C2 alkylidene and R⁷ is —OH, —OCH₃; or V₁ is a covalent bond and R⁷ is cyclopropyl, cyclopentyl, furyl or tetrahydrofuryl;
R¹³ is —OH, —CN, C1-C3 alkoxy, or NR¹¹R¹², where R¹¹ is —H or a C1-C3 alkyl group and R¹² is —H, an optionally substituted alkyl, or an optionally substituted non-aromatic heterocyclic group, or NR¹¹R¹² is an optionally substituted aromatic or non-aromatic nitrogen containing heterocyclic group;
R^{13a} is an optionally substituted non-aromatic heterocyclic group selected from N-pyrrolidinyl, N-piperidinyl, N-morpholinyl, N-piperazinyl, N-thiomorpholinyl, N-azetidinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl and 3-oxo-N-8-azabicyclo[3.2.1]octyl or N-8-azabicyclo[3.2.1]octyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl and thiadiazolyl;
Cy is [2,5]thienylene or [2,5]furanylene; and
R⁴ and each R⁵ are independently —H, halogen, C1-C3 alkyl, C1-C3 haloalkyl, C1-C3 alkoxy, or C1-C3 haloalkoxy.

14. The compound of claim 1 wherein the compound is represented by the following structural formula: 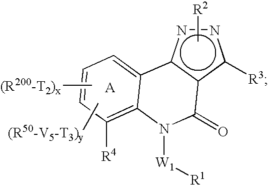
wherein:
Ring A is optionally substituted at any one or more substitutable ring carbon atoms; and
R¹ is —H, —CONR¹¹R¹², —COOR¹², fluoro, or a cycloalkyl optionally substituted with halo or alkyl and W₁ is a linear C1-C6 alkylidene chain or R¹ is —OR¹² W₁ is a linear C2-C6 alkylidene group wherein the alkylidene group represented by W₁ is optionally substituted with one or more —CH₃ or fluoro groups; or -W₁-R¹ is —H.

15. The compound of claim 14, wherein the compound is represented by a structural formula selected from: 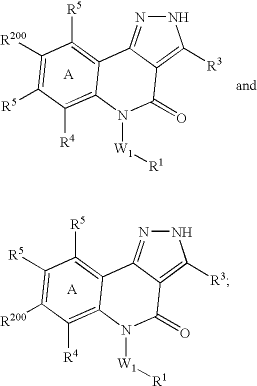
wherein:
R³ is —H, methyl, ethyl, n-propyl, iso-propyl, C1-C3 haloalkyl, or V₁-R⁷, wherein V₁ is a covalent bond or a C1-C2 alkylidene optionally substituted with one or two methyl groups or with a spiro cyclopropyl group; R⁷ is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, C3-C6 cycloalkyl, furyl, tetrahydrofuryl, N-piperazinyl, N′-alkyl-N-piperazinyl, N′-acyl-N-piperazinyl, N-pyrrolidyl, N-piperidinyl or N-morpholinyl;
each R⁵ is independently H, halogen, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)N(C1-C3 alkyl)₂, —NHC(O)O—(C1-C3 alkyl), —C(O)O—(C1-C3 alkyl), —NHC(O)NH₂, —NHC(O)NH(C1-C3 alkyl), —NHC(O)N(C1-C3 alkyl)₂, or —NHC(O)O—(C1-C3 alkyl);
R²⁰⁰ is —C≡CR²⁰¹, —CH═CHR²⁰¹, —C≡C—(C(R²⁰R²⁰))ₚR²⁰², or —CH═CH—(C(R²⁰R²⁰ ))ₚR²⁰²;
R²⁰¹ is —H, alkyl, haloalkyl, hydroxyalkyl, CO₂R⁵¹, or an optionally substituted aromatic group or non-aromatic heterocyclic group;
R²⁰² is —H, —CN, —OR⁵¹, —OC(O)NR⁵¹R⁵², —OC(O)R⁵¹, —NR⁵¹R⁵², —C(O)NR⁵¹R⁵², —N⁵¹C(O)R⁵¹, —NR⁵¹C(O)NR⁵¹R⁵², —NR⁵¹C(O)OR⁵¹, —NR⁵¹S(O)₂R^{x}, —S(O)₂NR⁵¹, —CO₂R⁵¹ or an optionally substituted aromatic group or non-aromatic heterocyclic group;
each R²⁰ is independently —H or C1-C3 alkyl;
R^{x} is alkyl or an optionally substituted aromatic group or non-aromatic heterocyclic group;
p is 1 or 2;
each substitutable ring nitrogen atom in an aromatic or non-aromatic heterocyclic group represented by R²⁰¹ or R²⁰² is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group;
each substitutable ring carbon atom of a non-aromatic heterocyclic group represented by R²⁰¹ or R²⁰² is optionally substituted with a C1-C3 alkyl group, hydroxy, fluoro, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 fluoroalkylamido, amino (C1-C3) alkyl, (C1-C3)alkylamino(C1-C3)alkyl, (C1-C3)dialkylamino(C1-C3)alkyl, hydroxy(C1-C3)alkyl, (C1-C3)alkoxy(C1-C3)alkyl;
each substitutable ring carbon atom of an aromatic group represented by R²⁰¹ or R²⁰² is optionally substituted with halo, hydroxy, cyano, C1-C3 alkyl, C1-C3 fluoroalkyl, C1-C3 alkoxy, C1-C3 fluoroalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, —NR′C(O)O—(C1-C3 alkyl)-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl); and
each R′ is independently hydrogen or a C1-C3 alkyl group.

16. The compound of claim 15, wherein:
R³ is methyl, or ethyl; or R³ is V₁-R⁷, wherein V₁ is a C1-C2 alkylidene and R⁷ is —OH, —OCH₃; or wherein V₁ is a covalent bond and R⁷ is cyclopropyl, cyclopentyl, furyl or tetrahydrofuryl;
R⁴ and each R⁵ are independently —H, halogen, —CH₃, halomethyl, —OCH₃, or haloalkoxy;
R²⁰¹ is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-azetidinyl 3-pyrrolidinyl, 3-piperidinyl, 3-morpholinyl, 3-thiomorpholinyl, 3-azetidinyl, 4-piperidinyl, tetrahydroquinolinyl, tetrahydroisoquinolinyl, N-tetrahydroquinolinyl, N-tetrahydroisoquinolinyl 3-oxo-N-8-azabicyclo[3.2.1]octyl or N-8-azabicyclo[3.2.1]octyl; and
R²⁰² is —CN, —OH, C1-C3 alkoxy, —NH₂, C1-C3 alkylamino, C1-C3 dialkylamino, an optionally substituted non-aromatic heterocyclic group selected from N-pyrrolidinyl, N-piperidinyl, N-morpholinyl, N-piperazinyl, N-thiomorpholinyl, 2-pyrrolidinyl, 2-piperidinyl, 2-piperazinyl, 2-morpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl, N-tetrahydroisoquinolinyl, 3-oxo-N-8-azabicyclo[3.2.1]octyl or N-8-azabicyclo[3.2.1]octyl.

17. The compound of claim 15, wherein the compound is represented by a structural formula selected from:

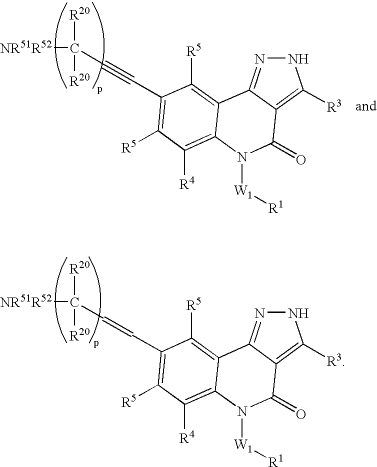

18. The compound of claim 17, wherein:
R³ is methyl, or ethyl; or R³ is V₁-R⁷, wherein V₁ is a C1-C2 alkylidene and R⁷ is —OH, —OCH₃; or wherein V₁ is a covalent bond and R⁷ is -cyclopropyl, cyclopentyl, furyl or tetrahydrofuryl;
R⁴ and each R⁵ are independently —H, halogen, —CH₃, halomethyl, —OCH₃, or haloalkoxy;
R²⁰⁰ is —C≡C—R²⁰³ or —C═CHR²⁰³;
R²⁰³ has the formula -V₆-R⁶⁰, -V₆-R⁶¹, -T₁₁-V₆-R⁶⁰, or -T₁₁-V₆-R⁶¹;
V₆ is a C1-C4 alkylidene, wherein V₆ is optionally substituted with alkyl, halo, haloalkyl, alkoxy, hydroxy, NR¹¹R¹² or oxo;
T₁₁ is —S(O)—, —S(O)₂—, —C(O)—, —C(O)O—, —C(O)N(R⁶)—, or —SO₂N(R⁶)—;
R⁶⁰ is —OR¹², —CN, —COOR¹², —NR¹¹R¹², —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —O—C(O)—OR¹², —OC(O)—NR¹¹R¹², —NR¹¹CO—CH(OR⁶²)—R¹², —NR¹¹CO—CH(N⁶²R⁶²)—R¹², —NR¹¹CO—(CH₂)_{z}CH⁶²R⁶²)—R¹², —OC(O)—CH(OR⁶²)—R¹², —OC(O)—CH(NR⁶²R⁶²)—R¹², —NR¹¹CO—C(R⁶²R⁶³)—OR¹², —NR¹¹CO—C(R⁶³R⁶³)—NR¹¹R¹², —OC(O)—C(R⁶³R⁶³)—OR¹², —OC(O)—C(R⁶³R⁶³)—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², —C(O)NR¹¹R¹², —NHC(O)NR¹¹R¹², or —C(═NR¹¹)—NR¹¹R¹²;
R⁶¹ is an optionally substituted nitrogen-containing heteroaromatic group or a nitrogen-containing non-aromatic heterocyclic group;
each R⁶² is independently —H, a C1-C3 alkyl group, —C(O)H, —C(O)—(C1-C3 alkyl), —C(O)NH₂, —C(O)NH—(C1-C3 alkyl), —C(O)N—(C1-C3 alkyl)₂, —C(O)O—(C1-C3 alkyl), —S(O)₂(C1-C3 alkyl) or —NR⁶²R⁶² taken together is a substituted or unsubstituted non-aromatic nitrogen-containing heterocyclic group;
each R⁶³ is independently —H, a C1-C3 alkyl group or —C(R⁶³R⁶³)— taken together is a C3-C8 cycloalkyl group; and
z is an integer from 1 to 4.

19. The compound of claim 18, wherein:
R⁶⁰ is —CN, —OR¹², —NR¹¹R¹², —NHC(O)R¹², —NHC(O)OR¹², —NHC(O)NR¹¹R¹², —NHC(O)OR¹², or —OC(O)R¹²;
R⁶¹ is an optionally substituted non-aromatic heterocyclic group selected from pyrrolidinyl, piperidinyl, morpholinyl, piperazinyl, azetidinyl, tetrahydrofuranyl, oxazolidinyl, thiomorpholinyl, tetrahydroquinolinyl, tetrahydroisoquinolinyl and azabicyclopentanyl, azabicyclohexanyl, azabicycloheptanyl, azabicyclononanyl, azabicyclodecanyl, diazabicyclohexanyl, diazabicycloheptanyl, diazabicyclooctanyl, diazabicyclononanyl, or diazabicyclodecanyl or an optionally substituted heteroaromatic group selected from imidazolyl, pyrrolyl, pyrazolyl, triazolyl, tetrazolyl, pyridinyl, pyrazinyl, pyrimidinyl, pyridazinyl, oxazolyl, isoxazolyl, oxadiazolyl, thiazolyl, isothiazolyl and thiadiazolyl;
each substitutable ring nitrogen atom of the group represented by R⁶¹ is optionally substituted with a C1-C3 alkyl, C1-C3 acyl, C1-C3 alkylsulfonyl, —OC(O)N(R′)₂, —NR′C(O)OR′, or —NR′C(O)N(R′)₂ group;
each substitutable ring carbon atom of a non-aromatic ring in the group represented by R⁶¹ is optionally substituted with a C1-C3 alkyl group, hydroxy, fluoro, oxo, —C(O)OH, —C(O)O(C1-C3 alkyl), C1-C3 alkoxy, —NH₂, C1-C3 alkylamino, C1-C3 dialkylamino, amido, C1-C3 alkylamido, C1-C3 fluoroalkylamido, amino (C1-C3) alkyl, (C1-C3)alkylamino(C1-C3)alkyl, (C1-C3)dialkylamino(C1-C3)alkyl, hydroxy(C1-C3)alkyl, (C1-C3)alkoxy(C1-C3)alkyl;
each substitutable ring carbon atom of an aromatic ring in the group represented by R⁶¹ is optionally substituted with halo, hydroxy, cyano, C1-C3 alkyl, C1-C3 fluoroalkyl, C1-C3 alkoxy, C1-C3 fluoroalkoxy, —NH₂, C1-C3 alkylamino, C1-C3 dialkylamino, —C(O)NH₂, —C(O)NH(C1-C3 alkyl), C(O)N(C1-C3 alkyl)₂, —NR′CO(C1-C3 alkyl), —NR′CO(C1-C3 haloalkyl), —NR′C(O)O(C1-C3 alkyl), —C(O)O(C1-C3 alkyl), —NR′C(O)NH₂, —NR′C(O)NH(C1-C3 alkyl), —NR′C(O)N(C1-C3 alkyl)₂, —NR′C(O)O—(C1-C3 alkyl)-SH, —S(C1-C3 alkyl), —NO₂, —S(O)₂H, —S(O)₂(C1-C3 alkyl), —SO₂N(R′)₂, —S(O)H, —S(O)(C1-C3 alkyl), —NR′S(O)₂NH₂, —NR′S(O)₂NH(C1-C3 alkyl), —NR′S(O)₂N(C1-C3 alkyl)₂, —NR′S(O)₂H or —NR′S(O)₂(C1-C3 alkyl); and
each R′ is hydrogen or a C1-C3 alkyl group.

20. The compound of claim 19, wherein:
R⁶⁰ is NH₂, C1-C3 alkylamino, or C1-C3 dialkylamino; and
R⁶¹ is an optionally substituted non-aromatic heterocyclic group selected from N-pyrrolidinyl, N-piperidinyl, N-morpholinyl, N-piperazinyl, N-azetidinyl, N-thiomorpholinyl, 2-pyrrolidinyl, 2-piperidinyl, 2-piperazinyl, 2-morpholinyl, 2-thiomorpholinyl, 3-pyrrolidinyl, 3-piperidinyl, 3-morpholinyl, 3-thiomorpholinyl, 4-piperidinyl, tetrahydroquinolinyl, tetrahydroisoquinolinyl, N-tetrahydroquinolinyl N-tetrahydroisoquinolinyl 3-oxo-N-8-azabicyclo[3.2.1]octyl or N-8-azabicyclo[3.2.1]octyl.

21. A compound selected from:
8-[3-(diethylamino)prop-1-yn-1-yl]-5-ethyl-3-methyl-2,5-dihydro-4H-pyrazolo[4,3-c]quinolin-4-one;
8-{3-[(2S,5S)-2,5-dimethylpyrrolidin-1-yl]prop-1-yn-1-yl}-5-ethyl-3-methyl-2,5-dihydro-4H-pyrazolo[4,3-c]quinolin-4-one;
5-ethyl-3-methyl-8-(3-pyrrolidin-1-ylprop-1-yn-1-yl)-2,5-dihydro-4H-pyrazolo[4,3-c]quinolin-4-one;
5-ethyl-3-methyl-8-[(1E)-3-pyrrolidin-1-ylprop-1-en-1-yl]-2,5-dihydro-4H-pyrazolo[4,3-c]quinolin-4-one;
8-[3-(diisopropylamino)prop-1-yn-1-yl]-5-ethyl-3-methyl-2,5-dihydro-4H-pyrazolo[4,3-c]quinolin-4-one;
8-{3-[(2R,6S)-2,6-dimethylpiperidin-1-yl]prop-1-yn-1-yl}-5-ethyl-3-methyl-2,5-dihydro-4H-pyrazolo[4,3-c]quinolin-4-one;
8-{3-[tert-butyl(isopropyl)amino]prop-1-yn-1-yl}-5-ethyl-3-methyl-2,5-dihydro-4H-pyrazolo[4,3-c]quinolin-4-one;
8-{(1E)-3-[(2S,5S)-2,5-dimethylpyrrolidin-1-yl]prop-1-en-1-yl}-5-ethyl-3-methyl-2,5-dihydro-4H-pyrazolo[4,3-c]quinolin-4-one;
8-[(1E)-3-(diethylamino)prop-1-en-1-yl]-5-ethyl-3-methyl-2,5-dihydro-4H-pyrazolo[4,3-c]quinolin-4-one;
8-[(1E)-3-(diisopropylamino)prop-1-en-1-yl]-5-ethyl-3-methyl-2,5-dihydro-4H-pyrazolo[4,3-c]quinolin-4-one; and
8-(5-{[(2S,5S)-2,5-dimethylpyrrolidin-1-yl]methyl}-2-thienyl)-5-ethyl-3-methyl-2,5-dihydro-4H-pyrazolo[4,3-c]quinolin-4-one.

22. A compound selected from:
8-[(1E)-3-(diisopropylamino)prop-1-en-1-yl]-5-ethyl-3-(2-methoxyethyl)-2,5-dihydro-4H-pyrazolo[4,3-c]quinolin-4-one; and
8-{(1E)-3-[(2S,5S)-2,5-dimethylpyrrolidin-1-yl]prop-1-en-1-yl}-5-ethyl-3-(2-hydroxyethyl)-2,5-dihydro-4H-pyrazolo[4,3-c]quinolin-4-one.

23. A method of treating a proliferative disorder in a subject comprising administering an effective amount of the Chk-1 inhibitor of claim 1.

24. The method of claim 23, wherein the proliferative disorder is a cancer.

25. The method of claim 24, wherein the cancer is one in which a checkpoint pathway has been mutated or upregulated.

26. The method of claim 25, wherein the Chk-1 inhibitor is administered in combination with another therapeutic agent.

27. The method of claim 26, wherein the Chk-1 inhibitor and the other therapeutic agent are administered as part of the same pharmaceutical composition.

28. The method of claim 27, wherein the Chk-1 inhibitor and the other therapeutic agent are administered as separate pharmaceutical compositions, and the Chk-1 inhibitor is administered prior to, at the same time as, or following administration of the other agent.

29. The method of claim 28, wherein the other therapeutic agent is an anticancer agent.

30. The method of claim 29, wherein the anticancer agent is selected from the group consisting of DNA damaging agents; cytotoxic agents; agents that disrupt cell replication; proteasome inhibitors; and NF-κB inhibitors.

31. The method of claim 30, wherein the anticancer agent is a DNA damaging agent.

32. The method of claim 31, wherein the DNA damaging agent is selected from the group consisting of radiation therapy, topoisomerase I inhibitors, topoisomerase II inhibitors, alkylating agents, DNA intercalators, and nucleoside mimetics.

33. A pharmaceutical composition comprising the compound of claim 1 and at least one pharmaceutically acceptable carrier or diluent.


SEQUENCE LISTING (2 sequences)

SEQ ID NO: 1 - protein, 14 aa, Artificial Sequence
  Chemically synthesized
ILSRRPSYRKILND

SEQ ID NO: 2 - protein, 11 aa, Artificial Sequence
  AMIDATION 11: Amido c-terminus
GLYRSPSMPEN